(19) United States
(12) Patent Application Publication
(10) Pub. No.: US 2025/0163501 A1
(43) Pub. Date: May 22, 2025

(54) METHODS, KITS, COMPOSITIONS, AND SYSTEMS FOR SPATIAL ANALYSIS

(71) Applicant: 10x Genomics, Inc., Pleasanton, CA (US)

(72) Inventors: Hardeep Pal Singh, Livermore, CA (US); David Sukovich, Oakland, CA (US); Augusto Manuel Tentori, Dublin, CA (US); Hanyoup Kim, Foster City, CA (US)

(21) Appl. No.: 18/834,805

(22) PCT Filed: Jan. 31, 2023
(86) PCT No.: PCT/US2023/011948

§ 371 Date: Jul. 31, 2024

Related U.S. Application Data
(60) Provisional application No. 63/341,181, filed on May 12, 2022.
(60) Provisional application No. 63/305,491, filed on Feb. 1, 2022.

(51) Int. Cl. C12Q 1/6841; C12Q 1/37

(52) U.S. Cl. CPC C12Q 1/6841; C12Q 1/37

(57) ABSTRACT

Provided herein are methods of analyzing an analyte in a fixed biological sample on a first substrate that has been stored for a long period of time, the method comprising: (a) hybridizing a first probe and a second probe to the analyte of the fixed biological sample; (b) coupling the first probe and the second probe, thereby generating a connected probe; (c) aligning the first substrate with a second substrate comprising an array, wherein the array comprises a plurality of capture probes, wherein a capture probe of the plurality of capture probes comprises: (i) a spatial barcode and (ii) a capture domain; (d) when the biological sample is aligned with at least a portion of the array, (i) releasing the connected probe from the analyte and (ii) migrating the connected probe from the biological sample to the array; and (e) hybridizing the connected probe to the capture domain.

### CROSS-REFERENCE TO RELATED APPLICATIONS

This application claims the benefit of priority to U.S. Provisional Patent Application No. 63/305,491, filed on Feb. 1, 2022, and U.S. Provisional Patent Application No. 63/341,181, filed on May 12, 2022. The content of each of those patent applications is incorporated herein by reference in their entireties.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Biological samples for spatial analysis are quality controlled by extraction of RNA followed by an RNA integrity analysis (e.g., RNA Integrity Number (RIN)). Spatial fragment distribution value (DV) is another method of measuring RNA integrity (e.g., degradation) in a biological sample, including fixed biological samples, and can also identify spatial patterns of degradation within a biological sample.

### SUMMARY

A fundamental tool in anatomical pathology for disease diagnosis is preserving tissues in the form of formalin-fixed paraffin-embedded (FFPE) samples. A major advantage of this type of sample is its ability to maintain the morphology and structure of cells within a tissue sample, which is the basis of disease diagnosis and biomarker detection. This advantage has made FFPE specimens a popular approach for long-term preservation of biological samples (e.g., tissue sections). However, since the crosslinks introduced through the fixation significantly affect the integrity of the nucleic acids within, their use is limited, especially in studies that involve gene expression analysis. Therefore, developing a workflow that enables determination of nucleic acid integrity (e.g., quality) in fixed (e.g., FFPE) biological samples will have a positive impact on both the research community and pathology departments.

Numerous studies have investigated and evaluated the integrity of fixed biological samples and their nucleic acid content. There remains a need to provide estimations of FFPE nucleic acids integrity as a function of their spatial distribution.

Despite potential drawbacks linked to fixed biological samples, several studies have shown that nucleic acids (e.g., RNA) derived from FFPE samples can still be used to generate transcriptome information comparable to fresh frozen biological samples. However, since RNA integrity varies in different fixed biological samples (e.g., FFPE), not all fixed biological samples can generate usable high-quality data. Thus, performing gene expression analysis on fixed biological samples with high degradation levels is most likely to fail in providing interpretable results. In order to avoid wasting reagents, money, and time associated with expression analysis methods, a quality control assay can determine whether an analysis method is likely to provide accurate data from a biological sample, including biological samples that have been fixed and/or stored at room temperature for long periods of time, such as months and years.

For example, determining RNA integrity in sub-areas of the tissue (e.g., in a spatial manner), including regions of interest, can facilitate the examination of fixed biological samples and ensure that sub-areas of the biological sample, including a region of interest, contain nucleic acids of sufficient quality to provide data for downstream analyses, including spatial transcriptomics. Thus, provided herein are methods for assessing the integrity of nucleic acids obtained from a fixed biological sample (e.g., a formalin-fixed paraffin-embedded biological sample). Also provided herein are methods for assessing the integrity of nucleic acids obtained from a fixed biological sample (e.g., a formalin-fixed paraffin-embedded biological sample), and then performing spatial analysis of one or more analytes in the biological sample when the nucleic acids are determined to be of sufficient integrity.

The present disclosure also features methods and systems for analyzing an analyte in a biological sample. Determining the spatial location and abundance of analytes (e.g., proteins, DNA, or RNA) within a biological sample leads to better understanding of spatial heterogeneity in various contexts, such as disease models. Described herein are methods for capturing probes and/or barcodes to a capture domain. In some instances, the techniques disclosed herein facilitate downstream processing, such as sequencing of the probes and/or barcodes bound to a capture domain.

In some examples, the methods and systems disclosed herein utilize RNA-templated ligation (RTL) for analyzing an analyte (e.g., RNA) in a biological sample. In some examples, RTL is used in combination with a “sandwich process,” wherein the proxy of an analyte (e.g., connected RTL probes) is transferred from a first substrate (e.g., containing the biological sample) to a second substrate (e.g., containing an array of polynucleotide capture probes) for further downstream processing. In some examples, analyte capture agents are used for analyzing an analyte (e.g., protein) in a biological sample. In some examples, the methods disclosed herein allow spatial analysis of two or more different types of analytes.

Thus provided herein are methods of analyzing an analyte in a fixed biological sample on a first substrate, wherein the fixed biological sample has been affixed to the first substrate for at least four months, the method comprising: (a) hybridizing a first probe and a second probe to the analyte of the fixed biological sample affixed to the first substrate for at least four months, wherein the first probe and the second probe each comprise a sequence that is substantially complementary to a first sequence and a second sequence of the analyte, respectively, and wherein the second probe further comprises a capture probe binding domain; (b) coupling the first probe and the second probe, thereby generating a connected probe; (c) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain; (d) when the biological sample is aligned with at least a portion of the array, (i) releasing the connected probe from the analyte and (ii) migrating the connected probe from the biological sample to the array; and (e) hybridizing the connected probe to the capture domain.

In some embodiments, the fixed biological sample has been affixed to the first substrate in contact with a mounting agent and a coverslip. In some embodiments, the mounting agent comprises glycerin, water-soluble mounting media, or a carbohydrate. In some embodiments, the coverslip is removed prior to the hybridizing step (a).

In some embodiments, the fixed biological sample has been affixed to the first substrate for at least six months. In some embodiments, the fixed biological sample has been affixed to the first substrate for at least one year. In some embodiments, the fixed biological sample has been affixed to the first substrate for at least two years. In some embodiments, the fixed biological sample has been affixed to the first substrate for at least three years.

In some embodiments, the fixed biological sample has been affixed to the first substrate at a temperature above −20° C. In some embodiments, the fixed biological sample has been affixed to the first substrate at a temperature above 4° C. In some embodiments, the fixed biological sample has been affixed to the first substrate at room temperature. In some embodiments, the fixed biological sample has been affixed to the first substrate at a temperature above room temperature.

Also provided herein are methods of analyzing an analyte in a fixed biological sample on a first substrate, the method comprising: (a) isolating a portion of the fixed biological sample on the first substrate; (b) determining the presence or absence of RNA of sufficient integrity in the portion of the fixed biological sample; (c) when RNA of sufficient integrity is present in the portion of the fixed biological sample, hybridizing a first probe and a second probe to the analyte, wherein the first probe and the second probe each comprise a sequence that is substantially complementary to a first sequence and a second sequence of the analyte, respectively, and wherein the second probe further comprises a capture probe binding domain; (d) coupling the first probe and the second probe, thereby generating a connected probe; (e) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain; (f) when the biological sample is aligned with at least a portion of the array, (i) releasing the connected probe from the analyte and (ii) migrating the connected probe from the biological sample to the array; and (g) hybridizing the connected probe to the capture domain.

In some embodiments, step (c) comprises contacting the fixed biological sample with the first probe and the second probe, and wherein upon the contacting, the first probe and the second probe hybridize to the analyte.

In some embodiments, determining the presence or absence of RNA of sufficient integrity comprises determining a spatial fragment distribution value (DV) number of the portion of the fixed biological sample. In some embodiments, the spatial fragment DV number of 30 or greater is indicative of the presence of RNA of sufficient integrity. In some embodiments, the spatial fragment DV number is above 30, above 40, above 50, above 60, or above 70, and is indicative of the presence of RNA of sufficient integrity. In some embodiments, determining the presence of RNA of sufficient integrity comprises determining an RNA integrity number (RIN) score of the portion of the fixed biological sample. In some embodiments, the RIN score of 6 or greater is indicative of the presence of RNA of sufficient integrity. In some embodiments, the RIN score of 7 or greater is indicative of the presence of RNA of sufficient integrity.

In some embodiments, the fixed biological sample is a formalin-fixed paraffin-embedded biological sample, a PFA fixed biological sample, or an acetone fixed biological sample. In some embodiments, the fixed biological sample is a fixed tissue sample. In some embodiments, the fixed biological sample is an FFPE tissue section, a PFA fixed tissue section, or an acetone fixed tissue section.

In some embodiments, the first probe and the second probe are on a contiguous nucleic acid sequence. In some embodiments, the first probe is on the 3′ end of the contiguous nucleic acid sequence. In some embodiments, the second probe is on the 5′ end of the contiguous nucleic acid sequence. In some embodiments, the first sequence and the second sequence are adjacent sequences of the analyte. In some embodiments, the first sequence and the second sequence are not adjacent to each other on the analyte. In some embodiments, the method further comprises extending the first probe to generate an extended first probe, thereby filling a gap between the hybridized first probe and the hybridized second probe. In some embodiments, the method further comprises generating an extended second probe using a polymerase, wherein the extended second probe comprises a sequence complementary to a sequence between the sequence hybridized to the first probe and the sequence hybridized to the second probe.

In some embodiments, the method further comprises hybridizing a third probe to the first probe and the second probe. In some embodiments, the third probe comprises: (i) a sequence that is at least 80%, at least 85%, at least 90%, at least 95%, at least 96%, at least 97%, at least 98%, or at least 99%, or 100% complementary to a portion of the first probe that hybridizes to the third probe; and (ii) a sequence that is at least 80%, at least 85%, at least 90%, at least 95%, at least 96%, at least 97%, at least 98%, or at least 99%, or 100% complementary to a portion of the second probe that hybridizes to the third probe.

In some embodiments, the coupling of the first probe and the second probe comprises ligating the first probe and the second probe, optionally wherein the ligating comprises use of a ligase. In some embodiments, the coupling of the first probe and the second probe comprises uses a ligase to couple: (i) the first probe and the extended second probe; or (ii) the extended first probe and the second probe. In some embodiments, the ligase is selected from a splintR ligase, a single stranded DNA ligase, or a T4 DNA ligase.

In some embodiments, the method further comprises amplifying the connected probe prior to the releasing step. In some embodiments, the amplifying comprises rolling circle amplification.

In some embodiments, the fixed biological sample is in contact with a reagent medium comprising a permeabilization agent and an agent for releasing the connected probe during the releasing step, thereby permeabilizing the fixed biological sample and releasing the connected probe from the analyte. In some embodiments, the agent for releasing the connected probe comprises a nuclease. In some embodiments, the nuclease comprises an RNase, optionally wherein the Rnase is selected from Rnase A, Rnase C, Rnase H, or Rnase I. In some embodiments, the permeabilization agent comprises a protease. In some embodiments, the protease is selected from trypsin, pepsin, elastase, or proteinase K. In some embodiments, the reagent medium further comprises a detergent. In some embodiments, the detergent is selected from sodium dodecyl sulfate (SDS), sarkosyl, saponin, Triton X-100™, or Tween-20™. In some embodiments, the reagent medium comprises less than 5 w/v % of a detergent selected from SDS and sarkosyl. In some embodiments, the reagent medium comprises at least 5% w/v % of a detergent selected from SDS and sarkosyl. In some embodiments, the reagent medium does not comprise sodium dodecyl sulfate (SDS) or sarkosyl. In some embodiments, the reagent medium further comprises polyethylene glycol (PEG). In some embodiments, the fixed biological sample and the array are contacted with the reagent medium for about 1 to about 60 minutes. In some embodiments, the fixed biological sample and the array are contacted with the reagent medium for about 30 minutes.

In some embodiments, the method further comprises determining (i) all or a part of the sequence of the connected probe, or a complement thereof, and (ii) the sequence of the spatial barcode, or a complement thereof, optionally wherein the method further comprises using the determined sequence of (i) and (ii) to determine the location and abundance of the analyte in the biological sample. In some embodiments, the determining comprises sequencing (i) all or a part of the sequence of the connected probe, or a complement thereof, and (ii) the sequence of the spatial barcode, or a complement thereof. In some embodiments, the sequence of the connected probe comprises the sequence of the spatial barcode or the reverse complement thereof, and a sequence corresponding to the analyte in the biological sample or reverse complement thereof.

In some embodiments, the capture domain of the capture probe comprises a poly(T) sequence. In some embodiments, the capture domain of the capture probe comprises a sequence complementary to the capture probe binding domain of the second probe. In some embodiments, the capture probe further comprises one or more functional domains, a unique molecular identifier (UMI), a cleavage domain, and combinations thereof.

In some embodiments, the analyte comprises RNA. In some embodiments, the RNA comprises mRNA.

In some embodiments, the method further comprises analyzing a different analyte in the biological sample. In some embodiments, the different analyte is a protein analyte. In some embodiments, the analyzing the different analyte comprises immunohistochemistry or immunofluorescence. In some embodiments, the protein analyte is an extracellular protein.

In some embodiments, the method further comprises analyzing a second analyte in a second fixed biological sample on a third substrate. In some embodiments, the second analyte is RNA. In some embodiments, the RNA is mRNA.

In some embodiments, the hybridizing of the first probe and the second probe to the analyte comprises contacting the fixed biological sample with a set of probe pairs, wherein a probe pair of the set of probe pairs comprises the first probe and the second probe.

In some embodiments, the method comprises: mounting the first substrate on a first member of a sample holder, the first member configured to retain the first substrate; mounting the second substrate on a second member of the sample holder, the second member configured to retain the second substrate; and operating an alignment mechanism of the sample holder to move the first member and/or the second member such that at least a portion of the biological sample is aligned with at least a portion of the array and the portion of the biological sample and the portion of the array contact the reagent medium.

In some embodiments, the first substrate and the second substrate are separated by a distance of less than 50 micrometers. In some embodiments, at least one of the first substrate and the second substrate further comprise a spacer. In some embodiments, after the first and second substrate is mounted on the sample holder, the spacer is disposed between the first substrate and second substrate and is configured to maintain the reagent medium within a chamber formed by the first substrate, the second substrate, and the spacer, and maintain a separation distance between the first substrate and the second substrate, the spacer positioned to at least partially surround an area on the first substrate on which the biological sample is disposed and/or the array disposed on the second substrate, wherein the area of the first substrate, the spacer, and the second substrate at least partially encloses a volume comprising the biological sample. In some embodiments, the chamber comprises a partially or fully sealed chamber. In some embodiments, the separation distance comprises a distance of at least 2 μm. In some embodiments, the separation distance comprises a distance between about 5 μm to 25 μm. In some embodiments, the first substrate comprises the spacer. In some embodiments, the second substrate comprises the spacer.

In some embodiments, the method further comprises delivering the reagent medium to the first substrate and/or the second substrate, wherein the delivering the reagent medium comprises delivering the reagent medium to a region of the spacer, the region outside an enclosed area of the second substrate, the enclosed area formed by the spacer.

In some embodiments, the method further comprises assembling the chamber, wherein assembling the chamber comprises positioning, responsive to the delivering, the first substrate at an angle such that a dropped side of the first substrate contacts at least a portion of the reagent medium when the first substrate and the second substrate are within a threshold distance along an axis orthogonal to the second substrate, the dropped side urging the reagent medium toward the three sides partially surrounding the fluid, and optionally wherein assembling the chamber further comprises positioning the first substrate and the second substrate in an approximately parallel arrangement relative to one another.

In some embodiments, the sample holder is configured to maintain an approximately parallel arrangement of the first substrate and the second substrate. In some embodiments, the sample holder further comprises an alignment mechanism coupled to the second member, the alignment mechanism comprising a linear actuator configured to move the second member along an axis orthogonal to the plane of the second member. In some embodiments, the linear actuator is configured to move the second member along the axis orthogonal to the plane of the second member at a velocity of at least 0.1 mm/sec. In some embodiments, the linear actuator is configured to move the second member along the axis orthogonal to the plane of the second member with an amount of force of at least 0.1 lbs.

Also provided herein are systems or kits for analyzing an analyte in a fixed biological sample, the system or the kit comprising: (a) a sample holder comprising a first member configured to retain a first substrate, a second member configured to retain a second substrate comprising an array, and an alignment mechanism configured to cause relative movement of the first support member and/or the second member such that at least a portion of the biological sample is aligned with at least a portion of the array, wherein the fixed biological sample is placed on the first substrate, and wherein the array comprises a plurality of capture probes, wherein a capture probe of the plurality of capture probes comprises: (i) a spatial barcode and (ii) a capture domain; (b) (b1) a first probe and a second probe, wherein the first probe and the second probe each comprise a sequence that is substantially complementary to a first sequence and a second sequence of the analyte, wherein the second probe comprises a capture probe binding domain, and wherein the first probe and the second probe are capable of being ligated together to form a connected probe; and/or (b2) a plurality of analyte capture agents, wherein an analyte capture agent of the plurality of analyte capture agents comprises an analyte binding moiety and a capture agent barcode domain, wherein the analyte binding moiety specifically binds to the analyte, and wherein the capture agent barcode domain comprises an analyte binding moiety barcode and a capture handle sequence; (c) reagents for determining the presence of RNA of sufficient integrity in the fixed biological sample; (d) a reagent medium comprising a permeabilization agent and optionally an agent for releasing the connected probe; and (e) instructions for performing any one of the methods described herein.

In some embodiments, the permeabilization agent is pepsin or proteinase K. In some embodiments, the agent for releasing the connected probe is an RNAse, optionally wherein the RNAse is selected from RNase A, RNase C, RNase H, or RNase I.

In some embodiments, the system or kit further comprises an alignment mechanism on the support device to align the first substrate and the second substrate. In some embodiments, the alignment mechanism is configured to maintain a separation distance between the first and second substrates when the first and second substrates are aligned, and wherein the separation distance is less than 50 microns. In some embodiments, at least one of the first substrate and the second substrate further comprise a spacer.

In some embodiments, after the first and second substrate being mounted on a sample holder, the spacer is disposed between the first substrate and second substrate and is configured to maintain the reagent medium within a chamber formed by the first substrate, the second substrate, and the spacer, and maintain a separation distance between the first substrate and the second substrate, the spacer positioned to at least partially surround an area on the first substrate on which the biological sample is disposed and/or the array disposed on the second substrate, wherein the area of the first substrate, the spacer, and the second substrate at least partially encloses a volume comprising the biological sample, optionally wherein the separation distance is less than 50 microns.

In some embodiments, the chamber comprises a partially or fully sealed chamber. In some embodiments, the separation distance is at least 2 μm. In some embodiments, the separation distance is between about 5 μm to 25 μm. In some embodiments, the first substrate comprises the spacer. In some embodiments, the second substrate comprises the spacer.

In some embodiments, the system or kit further comprises delivering the reagent medium to the first substrate and/or the second substrate, wherein the delivering the reagent medium comprises delivering the reagent medium to a region of the spacer, the region outside an enclosed area of the second substrate, the enclosed area formed by the spacer. In some embodiments, the system or kit further comprises assembling the chamber, wherein assembling the chamber comprises positioning, responsive to the delivering, the first substrate at an angle such that a dropped side of the first substrate contacts at least a portion of the reagent medium when the first substrate and the second substrate are within a threshold distance along an axis orthogonal to the second substrate, the dropped side urging the reagent medium toward the three sides partially surrounding the fluid, and optionally wherein assembling the chamber further comprises positioning the first substrate and the second substrate in an approximately parallel arrangement relative to one another.

In some embodiments, the sample holder is configured to maintain an approximately parallel arrangement of the first substrate and the second substrate. In some embodiments, the sample holder further comprises an alignment mechanism coupled to the second member, the alignment mechanism comprising a linear actuator configured to move the second member along an axis orthogonal to the plane of the second member. In some embodiments, the linear actuator is configured to move the second member along the axis orthogonal to the plane of the second member at a velocity of at least 0.1 mm/sec. In some embodiments, the linear actuator is configured to move the second member along the axis orthogonal to the plane of the second member with an amount of force of at least 0.1 lbs.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is an exemplary schematic of the workflow for RNA integrity assays for tissue sections mounted on slides.

FIG. 3 is an exemplary image of a slide mounted with human brain tumor tissue samples, wherein gaskets are used to form wells surrounding portions in the tissue samples.

FIG. 4 is an exemplary image of a slide mounted with mouse brain tissue samples, before (upper panel) and after (lower panel) removing a portion of the sample into a tube for assaying RNA integrity.

FIG. 5 is an exemplary image of a slide mounted with human spleen tissue samples, before (upper panel) and after (lower panel) removing a portion of the sample into a tube for assaying RNA integrity.

FIGS. 6A-6E show Ki67 expression in a first lymph node section. FIG. 6A shows protein expression in a defined area of the lymph node; FIG. 6B shows an expanded view of the box in 2A, showing Ki67 protein expression in a lymph follicle; FIG. 6C shows Ki67 mRNA gene expression is a defined area in a lymph node; FIG. 6D shows mRNA gene expression cluster expression; and FIG. 6E shows a UMAP plot in the eight different clusters in the defined area of the lymph node, where cluster 7 correlated with histological features of Ki67 position cells.

FIGS. 7A-7E show Ki67 expression in a second lymph node section. FIG. 7A shows protein expression in a defined area of the lymph node; FIG. 7B shows an expanded view of the box in FIG. 7A, showing Ki67 protein expression in a lymph follicle; FIG. 7C shows Ki67 mRNA gene expression is a defined area in a lymph node; FIG. 7D shows mRNA gene expression cluster expression; and FIG. 7E shows a UMAP plot in the eight different clusters in the defined area of the lymph node, where cluster 6 correlates with histological features of Ki67 positive cells.

FIGS. 8A-8G show exemplary protein expression patterns in a lymph node tissue section. FIG. 8A shows CCNB2 protein expression; FIG. 8B shows CD40 protein expression; FIG. 8C shows RGS13 protein expression; FIG. 8D shows FANCA protein expression; FIG. 8E shows MEF2B protein expression; FIG. 8F shows TK1 protein expression; and FIG. 8G shows MYBL2 protein expression.

FIGS. 9A-9H shows images of a human lymph node after five months of storage at room temperature. FIG. 9A shows H&E expression. FIG. 9B shows RGS13 expression. FIG. 9C shows LAMP3 expression. FIG. 9D shows CD5L expression. FIG. 9E shows mRNA gene expression cluster expression. FIG. 9F shows CCL17 expression. FIG. 9G shows CD19 expression. FIG. 9H shows FABP4 expression. “C” followed by a number indicates the cluster number.

FIGS. 10A-10F shows images of a human tonsil having tonsillitis after two months of storage at room temperature. FIG. 10A shows H&E expression. FIG. 10B shows RGS13 expression. FIG. 10C shows CCL21 expression. FIG. 10D shows mRNA gene expression cluster expression. FIG. 10E shows CXCL13 expression. FIG. 10F shows KRT15 expression.

FIG. 11 shows an exemplary schematic diagram depicting a sandwiching process.

FIG. 12A shows a perspective view of an example sample handling apparatus in a closed position.

FIG. 12B shows a perspective view of the example sample handling apparatus in an open position.

FIG. 13A shows an exemplary sandwiching process where a first substrate, including a biological sample, and a second substrate are brought into proximity with one another.

FIG. 13B shows a fully formed sandwich configuration creating a chamber formed from the one or more spacers, the first substrate, and the second substrate.

FIG. 14A shows the first substrate angled over (superior to) the second substrate.

FIG. 14B shows that as the first substrate lowers, and/or as the second substrate rises, the dropped side of the first substrate may contact the drop of the reagent medium.

FIG. 14C shows a full closure of the sandwich between the first substrate and the second substrate with the spacer contacting both the first substrate and the second substrate.

FIG. 15 shows a side view of the angled closure workflow.

FIG. 16 shows a top view of the angled closure workflow.

FIG. 17 is a schematic illustrating a cleavable capture probe.

FIG. 18 is a schematic diagram of an exemplary multiplexed spatially-barcoded feature.

FIG. 19 is a schematic diagram of an exemplary analyte capture agent.

FIG. 20 is a schematic diagram depicting an exemplary interaction between a feature-immobilized capture probe 2024 and an analyte capture agent 2026.

FIG. 21 is a graph showing number of genes detected per spot according to sequencing depth (sequencing reads per spot), as well as usable quality RNA as measured in each sample by DV200 score in archived sections stored at room temperature (RT). Sections were from breast cancer, ovarian cancer, and spleen (each stored for 11 months at RT), lymph node (two different sections, each stored for 6 months at RT), and tonsillitis samples (stored for 1 or 2 months at RT). Number at each line represents the DV200 score; time on right shows duration of time at RT; IF: sample was also stained using immunofluorescence.

FIG. 22A-22H shows representative images of an FFPE human breast cancer sample that was stored at RT for 11 months. FIG. 22A is an image of an H&E stain of the human breast cancer sample. FIG. 22B shows expression of AQP5 in the human breast cancer sample. FIG. 22C shows 10 different clusters that are differentially expressed in the human breast cancer sample. Expression of additional individual genes are shown in FIG. 22D (CCL19), FIG. 22E (FABP4), FIG. 22F (KRT81), FIG. 22G (IGLV3-1), and FIG. 22H (LBP).

FIGS. 23A, 23C-23F show representative images of an FFPE human ovarian cancer sample that was stored at RT for 11 months. FIG. 23A is an image of an H&E stain on the human ovarian cancer sample. FIG. 23B is a graph showing spatial fragment distribution value (DV) compared to spatial fragment DV score in archived sections stored at RT. Sections were from breast cancer, ovarian cancer, and spleen (each stored for 11 months at RT), lymph node (two different sections, each stored for 5 months at RT), and tonsillitis samples (stored for 1 or 2 months at RT). FIG. 23C shows 10 different clusters that are differentially expressed in the human ovarian cancer sample. Expression of additional individual genes are shown in FIG. 23D (MARCO), FIG. 23E (IGHG1), and FIG. 23F (VWF).

FIG. 24A is an image of an H&E stain on a human spleen sample, also showing 10 different clusters that are differentially expressed in the human spleen sample. FIG. 24B is a graph showing spatial fragment distribution value (DV) compared to spatial fragment DV score in archived sections stored at RT. FIG. 24C is a graph showing number of genes detected per spot according to sequencing depth (sequencing reads per spot) in archived sections stored at room temperature (RT). Sections were from breast cancer, ovarian cancer, and spleen (each stored for 11 months at RT), lymph node (two different sections, each stored for 5 months at RT), and tonsillitis samples (stored for 1 or 2 months at RT). FIG. 24D shows 10 different clusters that are differentially expressed in the human spleen sample. Expression of additional individual genes are shown in FIG. 24E (CD22) and FIG. 24F (FBLN1).

FIG. 25A is an image of an H&E stain on a human lymph node sample. FIG. 25B shows 10 different clusters that are differentially expressed in the human lymph node sample. FIG. 25C shows expression of RGS13 in the human lymph node sample. FIG. 25D is a graph showing spatial fragment distribution value (DV) compared to spatial fragment DV score in archived sections stored at RT. FIG. 25E is a graph showing number of genes detected per spot according to sequencing depth (sequencing reads per spot) in archived sections stored at room temperature (RT). Sections were from breast cancer, ovarian cancer, and spleen (each stored for 11 months at RT), lymph node (two different sections, each stored for 5 months at RT), and tonsillitis samples (stored for 1 or 2 months at RT).

FIG. 26A shows 10 different clusters that are differentially expressed in a human tonsillitis sample. Expression of additional individual genes are shown in FIG. 26B (CXCL13), FIG. 26C (CCL21), FIG. 26E (PCNA), FIG. 26F (RGS13), and FIG. 26G (KRT15). FIG. 26D is an image of immunofluorescent labelling of PCNA in the human tonsillitis sample.

FIG. 27A shows 10 different clusters that are differentially expressed in a human tonsillitis sample. Expression of additional individual genes are shown in FIG. 27B (CXCL13), FIG. 27C (CCL21), FIG. 27E (PCNA), FIG. 27F (RGS13), and FIG. 27G (KRT15). FIG. 27D is an image of immunofluorescent labelling of CD45RO in the human tonsillitis sample.

FIG. 28 shows mean #UMIs per spot obtained from archived sections stored at room temperature (RT), as well as their DV200 score. Sections were from breast cancer, ovarian cancer, and spleen (each stored for 11 months at RT), lymph node (two different sections, each stored for 6 months at RT), and tonsillitis samples (stored for 1 or 2 months at RT). IF: sample was also stained using immunofluorescence.

### DETAILED DESCRIPTION

### A. Spatial Analysis Methods

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

In some instance, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, semi-random, defined or combinations thereof, depending on the target analyte(s) of interest.

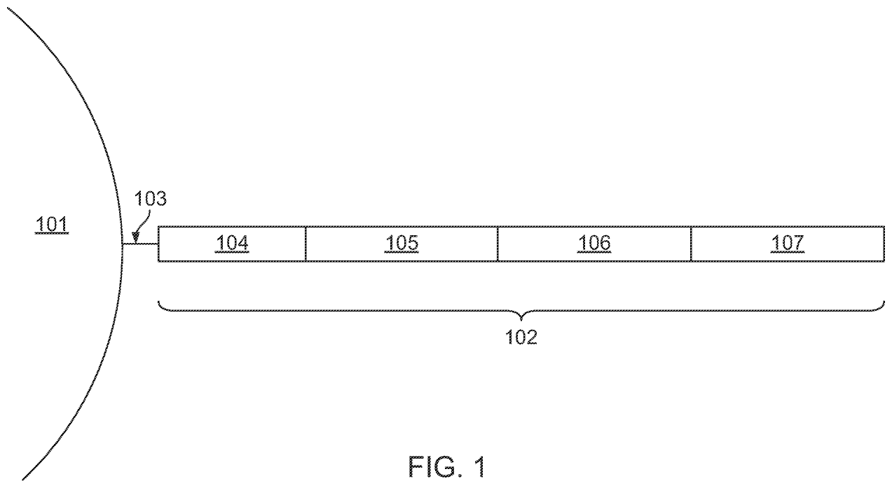

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

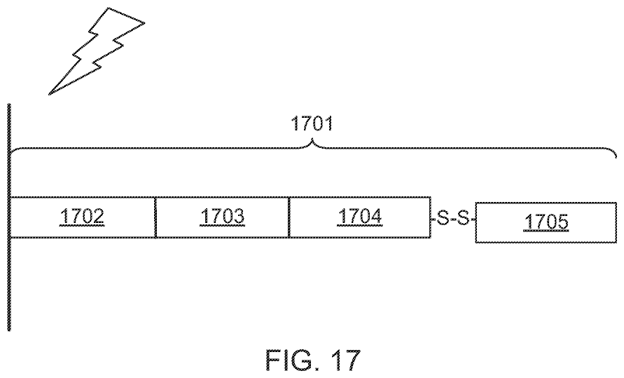

FIG. 17 is a schematic illustrating a cleavable capture probe, wherein the cleaved capture probe can enter into a non-permeabilized cell and bind to analytes within the sample. The capture probe 1701 contains a cleavage domain 1702, a cell penetrating peptide 1703, a reporter molecule 1704, and a disulfide bond (—S—S—). 1705 represents all other parts of a capture probe, for example a spatial barcode and a capture domain.

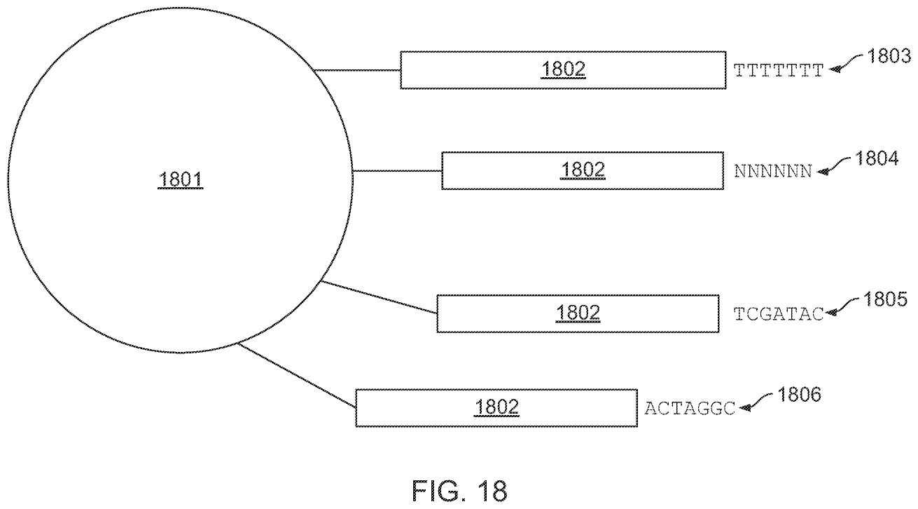

FIG. 18 is a schematic diagram of an exemplary multiplexed spatially-barcoded feature. In FIG. 18, the feature 1801 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 1802. One type of capture probe associated with the feature includes the spatial barcode 1802 in combination with a poly(T) capture domain 1803, designed to capture mRNA target analytes. A second type of capture probe associated with the feature includes the spatial barcode 1802 in combination with a random N-mer capture domain 1804 for gDNA analysis. A third type of capture probe associated with the feature includes the spatial barcode 1802 in combination with a capture domain complementary to a capture handle sequence of an analyte capture agent of interest 1805. A fourth type of capture probe associated with the feature includes the spatial barcode 1802 in combination with a capture domain that can specifically bind a nucleic acid molecule 1806 that can function in a CRISPR assay (e.g., CRISPR/Cas9). While only four different capture probe-barcoded constructs are shown in FIG. 18, capture-probe barcoded constructs can be tailored for analyses of any given analyte associated with a nucleic acid and capable of binding with such a construct. For example, the schemes shown in FIG. 18 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See. e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

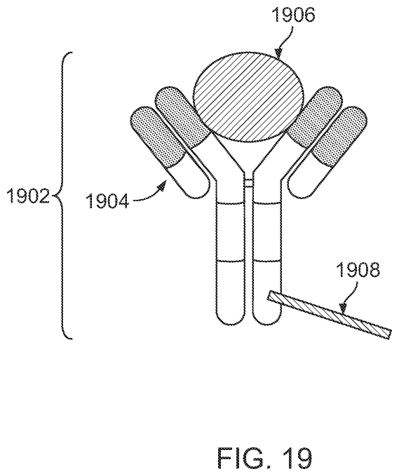

FIG. 19 is a schematic diagram of an exemplary analyte capture agent 1902 comprised of an analyte-binding moiety 1904 and an analyte-binding moiety barcode domain 1908. The exemplary analyte-binding moiety 1904 is a molecule capable of binding to an analyte 1906 and the analyte capture agent is capable of interacting with a spatially-barcoded capture probe. The analyte-binding moiety can bind to the analyte 1906 with high affinity and/or with high specificity. The analyte capture agent can include an analyte-binding moiety barcode domain 1908, a nucleotide sequence (e.g., an oligonucleotide), which can hybridize to at least a portion or an entirety of a capture domain of a capture probe. The analyte-binding moiety barcode domain 1908 can comprise an analyte binding moiety barcode and a capture handle sequence described herein. The analyte-binding moiety 1904 can include a polypeptide and/or an aptamer. The analyte-binding moiety 1904 can include an antibody or antibody fragment (e.g., an antigen-binding fragment).

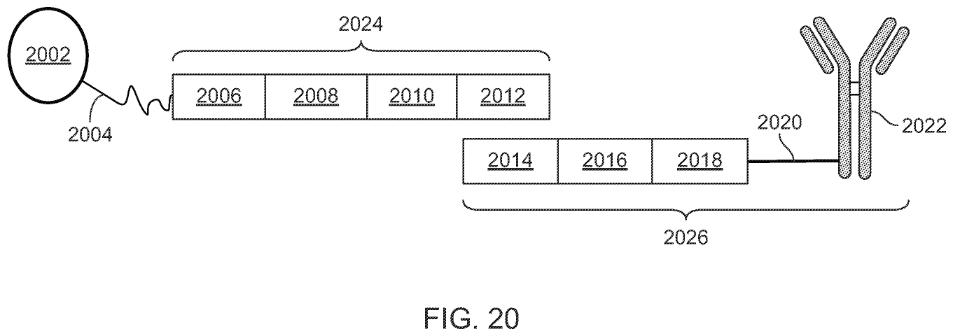

FIG. 20 is a schematic diagram depicting an exemplary interaction between a feature-immobilized capture probe 2024 and an analyte capture agent 2026. The feature-immobilized capture probe 2024 can include a spatial barcode 2008 as well as functional sequences 2006 and UMI 2010, as described elsewhere herein. The capture probe can also include a capture domain 2012 that is capable of binding to an analyte capture agent 2026. The analyte capture agent 2026 can include a functional sequence 2018, analyte binding moiety barcode 2016, and a capture handle sequence 2014 that is capable of binding to the capture domain 2012 of the capture probe 2024. The analyte capture agent can also include a linker 2020 that allows the capture agent barcode domain 2016 to couple to the analyte binding moiety 2022. As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNase A, RNase C, Rnase H, or RNase I). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

The sandwich process is described in PCT Patent Application Publication No. WO 2020/123320, which is incorporated by reference in its entirety.

### B. Spatial Analysis of Fixed Samples

A fundamental tool in anatomical pathology is preserving tissues in the form of formalin-fixed paraffin-embedded (FFPE) samples. A major advantage of this type of sample is its ability to maintain the morphology and structure of cells within a tissue sample, which is the basis of disease diagnosis and biomarker detection. This advantage has made FFPE specimens a popular approach for long-term preservation of biological samples (e.g., tissue sections). However, since the crosslinks introduced through the fixation significantly affect the integrity of the nucleic acids within, their use is limited, especially in studies that involve gene expression analysis. These issues can be particularly acute for FFPE specimens that have been stored for long periods of time, e.g., pre-sectioned FFPE samples that have been stored on slides. Therefore, there exists a need for methods, systems, and kits for processing such samples such that they can benefit from spatial analysis methodologies disclosed herein. Furthermore, developing a workflow that enables determination of nucleic acid integrity (e.g., quality) in fixed (e.g., FFPE) biological samples will have a positive impact on both the research community and pathology departments.

Despite potential drawbacks linked to fixed biological samples, several studies have shown that nucleic acids (e.g., RNA) derived from FFPE samples can still be used to generate transcriptome information comparable to fresh frozen biological samples. However, since RNA integrity varies in different fixed biological samples (e.g., FFPE), not all fixed biological samples can generate usable high-quality data. Thus, performing gene expression analysis on fixed biological samples with high degradation levels are most likely to fail in providing interpretable results. To avoid wasting reagents and time associated with expression analysis methods, a quality control assay can determine whether an analysis method will provide accurate data from a biological sample.

For example, determining RNA integrity in sub-areas of the tissue, including regions of interest, can facilitate the examination of fixed biological samples and ensure that sub-areas of the biological sample, including a region of interest, contain nucleic acids of sufficient quality to provide data for downstream analyses, including spatial transcriptomics.

Provided herein are methods for assessing the integrity of nucleic acids obtained from a fixed biological sample (e.g., a formalin-fixed paraffin-embedded biological sample). Some embodiments of any of the methods described herein can include the generation of a fragment distribution number (e.g., value). Some embodiments of any of the methods described herein include determining a spatial fragment distribution number (e.g., value) for a fixed biological sample. Some embodiments of any of the methods described herein can include the generation of a spatial fragment distribution heat map. Some embodiments of any of the methods described herein include determining an RNA integrity number (e.g., value) of a fixed biological sample.

A limitation of the spatial analysis methodologies (e.g., array-based spatial analysis methodologies) for some fixed samples is that integrity of RNA and other analytes for fixed samples (e.g., FFPE samples) can be lower than a fresh sample, particularly for FFPE tissue sections that have been stored (e.g., affixed to substrates such as glass slides) for long periods of time. However, the potential value of such analytical methods as applied to these samples can be significant. There exists a need for methods, systems, and kits for processing such samples such that they can benefit from spatial analysis methodologies disclosed herein.

Provided herein are methods to evaluate RNA integrity (e.g., using RIN, DV number (e.g., DV200), or other metrics) of a fixed biological sample by performing a portion of the integrity assay directly on a slide containing the fixed biological sample and sacrificing only a portion of the sample to do so. After evaluating the quality of the RNA using a portion of the fixed biological sample, the rest of the fixed biological sample can be utilized for the desired spatial assay, such as a sandwiching assay described herein. In some embodiments, a portion of the fixed biological sample can be scraped into a separate container (e.g., a tube), followed by RNA extraction, and optionally, quantitation. Subsequently, the rest of the fixed biological sample can be utilized for the desired spatial assay, such as a sandwiching assay described herein.

Provided herein are methods of analyzing an analyte in a fixed biological sample on a first substrate, wherein the fixed biological sample has been affixed to the first substrate for at least four months, the method including (a) hybridizing a first probe and a second probe to the analyte of the fixed biological sample affixed to the first substrate for at least four months, wherein the first probe and the second probe each comprise a sequence that is substantially complementary to a first sequence and a second sequence of the analyte, respectively, and wherein the second probe further comprises a capture probe binding domain; (b) coupling the first probe and the second probe, thereby generating a connected probe; (c) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain; (d) when the biological sample is aligned with at least a portion of the array, (i) releasing the connected probe from the analyte and (ii) migrating the connected probe from the biological sample to the array; and (e) hybridizing the connected probe to the capture domain.

Spatially determining RNA integrity in a biological sample, sub-areas of the biological sample, or regions of interest in a biological sample can facilitate the examination of fixed biological samples and ensure that sub-areas of the biological sample, including regions of interest, contain nucleic acids of sufficient integrity (e.g., quality) to provide data for downstream analyses, including spatial transcriptomics. Thicker tissues can have regions of variable RNA quality within a fixed tissue section due to variable penetration of fixative. The disclosed methods can facilitate identification of regions, within a fixed tissue sample, which have better RNA quality over others. In some embodiments, serial sections from the same tissue sample can then be used for spatial analysis on the selected region.

Thus, disclosed herein are methods involving assaying nucleic acid integrity in a first portion of a fixed biological sample (e.g., FFPE tissue section), and if the nucleic acid is of sufficient integrity, performing spatial analysis on a second portion or the remainder of the fixed biological sample. In some embodiments, the methods can involve assaying nucleic acid integrity in a first section of a fixed biological sample (e.g., FFPE tissue section), and if the nucleic acid is of sufficient integrity, performing spatial analysis on a second (e.g., serial) section of the fixed biological sample (e.g., in a region corresponding to an area of interest).

Also provided herein are methods for assessing the integrity (e.g., quality) of nucleic acids from a biological sample. In some embodiments, the biological sample is a fixed biological sample (e.g., formalin-fixed paraffin-embedded biological sample (FFPE), paraformaldehyde (PFA) fixed, acetone fixed, etc.). In some embodiments, assessing the integrity of the nucleic acids includes determining fragment distribution number (e.g., a spatial fragment distribution number (e.g., value)). In some embodiments, assessing the integrity of nucleic acids from a biological sample includes generating a spatial fragment distribution heat map. In some embodiments, assessing the integrity of nucleic acids in a biological sample include one or more detectable probes for a ribosomal RNA (rRNA). In some embodiments, the one or more detectable probes are probes for 18S rRNA. In some embodiments, the one or more detectable probes are for 28S ribosomal RNA. In some embodiments, assessing the integrity of the nucleic acids includes determining a RNA integrity number (e.g., value).

Also provided herein are methods of analyzing an analyte in a fixed biological sample on a first substrate, the method including (a) isolating a first portion of the fixed biological sample on the first substrate; (b) determining the presence or absence of RNA of sufficient integrity in the first portion of the fixed biological sample; (c) when RNA of sufficient integrity is present in the first portion of the fixed biological sample, hybridizing a first probe and a second probe to the analyte in a second portion of the fixed biological sample, wherein the first probe and the second probe each comprise a sequence that is substantially complementary to a first sequence and a second sequence of the analyte, respectively, and wherein the second probe further comprises a capture probe binding domain; (d) coupling the first probe and the second probe, thereby generating a connected probe; (e) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain; (f) when the biological sample is aligned with at least a portion of the array, (i) releasing the connected probe from the analyte and (ii) migrating the connected probe from the biological sample to the array; and (g) hybridizing the connected probe to the capture domain.

### (a) Fixed Biological Sample

In some embodiments, the fixed biological sample is a formalin-fixed paraffin-embedded biological sample, a PFA fixed biological sample, or an acetone fixed biological sample. In some embodiments, the fixed biological sample is a fixed tissue sample. In some embodiments, the fixed biological sample is an FFPE tissue section, a PFA fixed tissue section, or an acetone fixed tissue section. Any suitable fixative or fixation methods (e.g., embedding materials) can be used, including for example, ethanol, methanol, paraformaldehyde or formaldehyde. In some embodiments, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). In some embodiments, the biological sample is an FFPE biological sample. For example, the biological sample can be fixed in a suitable fixative, typically formalin, and embedded in melted paraffin wax. The wax block can be cut on a microtome to yield a thin slice of paraffin containing the biological sample. The biological sample can be applied to a substrate, air dried, and heated to cause the specimen to adhere to the glass slide. Residual paraffin can be dissolved with a suitable solvent, typically xylene, toluene, or others. These deparaffinizing solvents can be removed with washing and/or dehydrating reagents prior to staining. Sliced biological samples can be prepared from frozen specimens, fixed briefly in 10% formalin, and infused with a dehydrating reagent.

The biological sample as used herein can be any suitable biological sample described herein or known in the art. In some embodiments, the biological sample is a tissue. In some embodiments, the tissue sample is a solid tissue sample. In some embodiments, the biological sample is a tissue section. In some embodiments, the tissue is flash-frozen and sectioned. Any suitable methods described herein or known in the art can be used to flash-freeze and section the tissue sample. In some embodiments, the biological sample, e.g., the tissue, is flash-frozen using liquid nitrogen before sectioning. In some embodiments, the sectioning is performed using cryosectioning. In some embodiments, the methods further comprise a thawing step, after the cryosectioning.

In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probe oligonucleotides that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples.

In some embodiments, the fixed biological sample has been affixed to the first substrate in contact with a mounting agent and a coverslip. In some embodiments, the mounting agent comprises glycerin, water-soluble mounting media, or a carbohydrate. In some embodiments, the coverslip is removed prior to the hybridizing of the first probe and the second probe to the analyte. In some embodiments, the fixed biological sample has been affixed to the first substrate for less than four months (e.g., less than one month, less than two months, or less than three months). In some embodiments, the fixed biological sample has been affixed to the first substrate for at least one week, at least two weeks, at least one month, or at least two months. In some embodiments, the fixed biological sample has been affixed to the first substrate for at least four months (e.g., at least five months, at least six months, at least seven months, at least eight months, at least nine months, at least ten months, at least eleven months, or at least twelve months). In some embodiments, the fixed biological sample has been affixed to the first substrate for at least one year (e.g., at least two years, at least three years, at least four years, or at least five years). In some embodiments, the methods disclosed herein provide surprising and unexpected results wherein RNA of sufficient integrity is present in the fixed biological sample that has been stored for at least four months (e.g., at least five months, at least six months, at least seven months, at least eight months, at least nine months, at least ten months, at least eleven months, at least twelve months, at least two years, at least three years, at least four years, or at least five years).

In some embodiments, the fixed biological sample has been affixed to the first substrate at a temperature above −20° C. (e.g., above −18° C., above −16° C., above −14° C., above −12° C., above −10° C., above −8° C., above −6° C., above −4° C., above −2° C., above 0° C., or above 2° C.). In some embodiments, the fixed biological sample has been affixed to the first substrate at a temperature above 4° C. (e.g., above 6° C., or above 8° C., above 10° C., or above 12° C., above 14° C., or above 16° C., above 18° C., or above 20° C., above 22° C., or above 24° C., or above 26° C.). In some embodiments, the fixed biological sample has been affixed to the first substrate at about room temperature, wherein room temperature refers to a temperature around 20-25° C. In some instances, the temperature is about 25° C. In some instances, the temperature ranges from about 4° C. to about 40° C. In some instances, the temperature ranges from about 15° C. to about 35° C. In some instances, the temperature ranges from about 20° C. to about 30° C. In some instances, the temperature ranges from about 20° C. to about 25° C. In some embodiments, the fixed biological sample has been affixed to the first substrate at a temperature above room temperature (e.g., above about 25° C.).

In some embodiments, the fixed biological sample affixed to the first substrate has been stored at a temperature above −20° C. (e.g., above −18° C., above −16° C., above −14° C., above −12° C., above −10° C., above −8° C., above −6° C., above −4° C., above −2° C., above 0° C., or above 2° C.). In some embodiments, the fixed biological sample affixed to the first substrate has been stored at a temperature above 4° C. (e.g., above 6° C., or above 8° C., above 10° C., or above 12° C., above 14° C., or above 16° C., above 18° C., or above 20° C., above 22° C., or above 24° C., or above 26° C.). In some embodiments, the fixed biological sample affixed to the first substrate has been stored at about room temperature, wherein room temperature refers to a temperature around 20-25° C.

In some embodiments, the paraffin-embedding material can be removed (e.g., deparaffinization) from the biological sample (e.g., tissue section) by incubating the biological sample in an appropriate solvent (e.g., xylene), followed by a series of rinses (e.g., ethanol of varying concentrations), and rehydration in water. In some embodiments, the biological sample can be dried following deparaffinization. In some embodiments, after the step of drying the biological sample, the biological sample can be stained (e.g., H&E stain, any of the variety of stains described herein). In some embodiments, after staining the biological sample, the sample can be imaged.

After an FFPE biological sample has undergone deparaffinization, the FFPE biological sample can be further processed. For example, FFPE biological samples can be treated to remove formaldehyde-induced crosslinks (e.g., decrosslinking). In some embodiments, de-crosslinking the formaldehyde-induced crosslinks in the FFPE biological sample can include treating the sample with heat. In some embodiments, decrosslinking the formaldehyde-induced crosslinks can include performing a chemical reaction. In some embodiments, decrosslinking the formaldehyde-induced crosslinks, can include treating the sample with a permeabilization reagent. In some embodiments, decrosslinking the formaldehyde-induced crosslinks can include heat, a chemical reaction, and/or permeabilization reagents.

In some embodiments, decrosslinking formaldehyde-induced crosslinks can be performed in the presence of a buffer. For example, the buffer can be Tris-EDTA (TE) buffer. In some embodiments, the TE buffer has a pH of about 7.0 to about 9.0, about 7.1 to about 8.9, about 7.2 to about 8.8, about 7.3 to about 8.7, about 7.4 to about 8.6, about 7.5 to about 8.5, about 7.6 to about 8.4, about 7.7 to about 8.3, about 7.8 to about 8.2, about 7.9 to about 8.1, or about 8.0.

In some embodiments, the TE buffer has a temperature of about 60° C. to about 80° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78, about 79° C., or about 80° C.

In some embodiments, the fixed biological sample can be contacted with TE buffer for about 10 minutes to about 200 minutes, about 10 minutes to about 190 minutes, about 10 minutes to about 180 minutes, about 10 minutes to about 170 minutes, about 10 minutes to about 160 minutes, about 10 minutes to about 160 minutes, about 10 minutes to about 150 minutes, about 10 minutes to about 140 minutes, about 10 minutes to about 130 minutes, about 10 minutes to about 120 minutes, about 10 minutes to about 110 minutes, about 10 minutes to about 100 minutes, about 10 minutes to about 90 minutes, about 10 minutes to about 80 minutes, about 10 minutes to about 70 minutes, about 10 minutes to about 60 minutes, about 10 minutes to about 50 minutes, about 10 minutes to about 40 minutes, about 10 minutes to about 30 minutes, or about 10 minutes to about 20 minutes.

In some embodiments, the fixed biological sample can be contacted with TE buffer that has a temperature of about 65° C. to about 75° C., and is contacted with the fixed biological sample for about 30 minutes to about 90 minutes. In some embodiments, the TE buffer can have a temperature of about 70° C., a pH of about 8.0, and can be contacted with the fixed biological sample for about 60 minutes.

After decrosslinking the formaldehyde crosslinks (e.g., decrosslinking) in the fixed biological sample (e.g., FFPE tissue section, PFA tissue section, acetone tissue section), the biological sample can be permeabilized (e.g., permeabilized by any of the variety of methods described herein). In some embodiments, the fixed biological sample can be permeabilized with a protease. In some embodiments, the protease can be pepsin. In some embodiments, the protease can be proteinase K. In some embodiments, the protease can be pepsin and proteinase K. In some embodiments, the fixed biological sample can be permeabilized with a protease for about 10 minutes to about 60 minutes.

In some embodiments, the thickness of the biological sample (e.g., tissue section), for use in the methods described herein may be dependent on the method used to prepare the sample and the physical characteristics of the tissue. Thus, any suitable section thickness can be used. In some embodiments, the thickness of the biological sample section is at least 0.1 m, further preferably at least 0.2, 0.3, 0.4, 0.5, 0.7, 1.0, 1.5, 2, 3, 4, 5, 6, 7, 8, 9, or 10 m. In some embodiments the thickness of the biological sample section is at least 10, 11, 12, 13, 14, 15, 20, or 30 μm. In some embodiments, the thickness of the biological sample is 5-12 μm.

The biological sample, e.g., tissue sample, can be stained, and imaged prior, during, and/or after any step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the biological sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a human sample. In some instances, the sample is a human breast tissue sample. In some instances, the sample is a human brain tissue sample. In some embodiments, the sample can be derived from skin, brain, breast, lung, liver, kidney, prostate, tonsil, thymus, testes, bone, lymph node, ovary, eye, heart, or spleen. In some embodiments, the sample is an embryo sample. The embryo sample can be a non-human embryo sample. In some instances, the sample is a mouse embryo sample.

In some embodiments, releasing step comprises contacting the fixed biological sample with a reagent medium comprising a permeabilization agent and an agent for releasing the connected probe, thereby permeabilizing the fixed biological sample and releasing the connected probe from the analyte.

In some embodiments, the agent for releasing the connected probe comprises a nuclease. In some embodiments, the nuclease comprises an RNase, optionally wherein the RNase is selected from RNase A, RNase C, RNase H, or RNase I. In some embodiments, the permeabilization agent comprises a protease. In some embodiments, the protease is selected from trypsin, pepsin, elastase, or proteinase K. In some embodiments, the reagent medium further comprises a detergent. In some embodiments, the detergent is selected from sodium dodecyl sulfate (SDS), sarkosyl, saponin, Triton X-100™, or Tween-20™. In some embodiments, the reagent medium comprises less than 5 w/v % of a detergent selected from SDS and sarkosyl. In some embodiments, the reagent medium comprises at least 5% w/v % of a detergent selected from SDS and sarkosyl. In some embodiments, the reagent medium does not comprise sodium dodecyl sulfate (SDS) or sarkosyl. In some embodiments, the reagent medium further comprises polyethylene glycol (PEG).

In some embodiments, the biological sample and the array are contacted with the reagent medium for about 1 to about 60 minutes. In some embodiments, the biological sample and the array are contacted with the reagent medium for about 30 minutes.

(b) Extracting Nucleic Acids from Fixed Biological Samples

In some embodiments, determining the presence or absence of nucleic acid of sufficient integrity involves first extracting or isolating the nucleic acid (e.g., RNA) from a fixed biological sample (e.g., FFPE tissue section) or a portion thereof. In particular, methods of extracting RNA from FFPE samples are known in the art and reagents are commercially available. Suitable commercial kits for extracting or isolating RNA from FFPE samples include, but are not limited to, FFPE RNA purification Kit (Norgen), RNeasy FFPE kit (Qiagen, Catalog ID No.: 73504), AllPrep DNA/RNA FFPE kit (Qiagen), High Pure FFPE RNA Micro Kit (Roche), and PureLink FFPE RNA Isolation Kit (ThermoFisher).

### (c) Determining RNA Integrity

In some embodiments, determining the presence or absence of RNA of sufficient integrity comprises determining a spatial fragment distribution value (DV) number of the fixed biological sample. In some embodiments, the spatial fragment DV number of 30 or greater is indicative of the presence of RNA of sufficient integrity. In some embodiments, the spatial fragment DV number of 50 or greater is indicative of the presence of RNA of sufficient integrity.

In some embodiments, determining the presence of RNA of sufficient integrity comprises generating an RNA integrity number (RIN) score of the fixed biological sample. In some embodiments, the RIN score comprises a score between 1 and 10, with 1 being the most degraded RNA profile and 10 being the most intact. In some embodiments, a RIN score of 6 or greater is indicative of the presence of RNA of sufficient integrity (e.g., such that a spatial analysis assay is then performed on the sample). In some embodiments, a RIN score of 7 or greater (e.g., 8, 9, 10) is indicative of the presence of RNA of sufficient integrity (e.g., such that a spatial analysis assay is then performed on the sample).

RIN score of an RNA sample can be determined using any appropriate method in the art, such as, but not limited to, the Agilent Bioanalyzer system. To determine the RIN, the Bioanalyzer instrument software uses an algorithm that takes into account the entire electrophoretic trace of the RNA, not just the ratio of 28S and 18S rRNAs. The ratio of 28S and 18S rRNA peaks is also provided. As RNA degradation becomes more apparent, peak heights for the 28S and 18S rRNA peaks decrease, while smaller or degraded RNA peaks become more prominent. The 28S and 18S peaks can be hardly visible in RNA samples with significant degradation.

As used herein, “spatial fragment distribution value (DV)” refers to a measurement of nucleic acid integrity in a biological sample (e.g., FFPE biological sample) obtained from a spatial fragment DV assay. An exemplary use of a DV assay to assess RNA quality is described in Zhao, Y., et al., Robustness of RNA sequencing on older formalin-fixed paraffin-embedded tissue from high-grade ovarian serous adenocarcinomas. PloS One, 14: e0216050 (2019).

Fragment distribution values (DV) of an RNA sample can be calculated to estimate the percentage of fragments within a sample having a certain length. For example, DV can be calculated to estimate the percentage of fragments longer than 200 nt (DV200), 150 nt (DV150), 100 nt (DV100), 50 nt (DV50), or any desired length. Scores indicative of high quality can be obtained with the DV200 even for samples exhibiting weak 18S and 28S peaks. In some embodiments, the fragment DV score can be represented as a number from 1 to 100. Thus, for example, an RNA sample can be determined to have a fragment DV200 score of from 1 to 100. In some embodiments, an RNA sample can be determined to have a fragment DV150 score of from 1 to 100. In some embodiments, an RNA sample can be determined to have a fragment DV100 score of from 1 to 100. In some embodiments, an RNA sample can be determined to have a fragment DV50 score of from 1 to 100.

In some embodiments, a fragment DV score (e.g., DV200, DV150, DV100, DV50) is determined using an appropriate method in the art, such as, but not limited to, the Agilent Tapestation system. The Agilent TapeStation system is an automated electrophoresis solution for the sample quality control of DNA and RNA samples. The system integrates an instrument, data processing software, reagents, and ScreenTape devices specific for DNA and RNA. In some instances, the DV score (e.g., DV200, DV150, DV100, DV50) is considered of sufficient integrity if it is above 30. In some instances, the DV score (e.g., DV200, DV150, DV100, DV50) is considered of sufficient integrity if it is above 50.

A spatial fragment DV can be represented in multiple ways. For example, a spatial fragment DV can be represented as a “spatial fragment DV number” from 1 to 100. In some embodiments, the spatial fragment DV number is generated by detecting one or more detectable probes specifically bound to an extended capture probe (e.g., an extended capture probe generated by using rRNA as a template), or a complement thereof. The one or more detectable probes can be designed to detect different locations of the extended capture probe, or complement thereof, which can represent the integrity of the nucleic acid in the biological sample. A spatial fragment DV can also be represented as a “spatial fragment DV heat map” that can indicate a spatial fragment DV at one or more locations in the biological sample. In some embodiments, a spatial fragment DV heat map can be generated by detecting one or more detectable probes specifically bound to an extended capture probe, imaging the biological sample (e.g., FFPE, PFA, acetone fixed biological sample), disassociating one or more detectable probes, and repeating the process. The images obtained by detecting the one or more detectable probes (e.g., a first detectable probe, a second detectable probe, or more) can be compared and viewed as a spatial fragment (DV) heat map. In some embodiments, the detection of the detectable label hybridized to an extended capture probe as a percentage of total area of the fixed biological sample being evaluated is used to determine the first spatial fragment DV number.

The analyte in the nucleic acid integrity assay (e.g., spatial fragment DV assay) refers to a nucleic acid present in the biological sample. In some embodiments, the analyte is RNA. In some embodiments, the analyte is a coding RNA. In some embodiments, the analyte is a non-coding RNA. In some embodiments, the RNA is messenger RNA (mRNA) or ribosomal RNA (rRNA). In some embodiments, the RNA is double-stranded RNA. In some embodiments, the RNA is single-stranded RNA. In some embodiments, the RNA is a circular RNA. It is contemplated that as long as an RNA is at least 200 nucleotides long and is abundant in a cell it could serve as a template for measuring RNA integrity. In some instances, RNA integrity is examined by calculating the DV200 score. The “DV200” is the percentage of RNA fragments >200 nucleotides. In some instances, the DV200 score is considered of sufficient integrity if it is above 50 (e.g., above 52, above 54, above 56, above 58, above 60, above 62, above 64, above 66, above 68, above 70, above 72, above 74, above 76, above 78, or above 80). In some instances, the DV200 score is considered of sufficient integrity if it is above 30 (e.g., above 32, above 34, above 36, above 38, above 40, above 42, above 44, above 46, or above 48).

In some embodiments, a fixed biological sample is contacted with a substrate including a plurality of capture probes (e.g., any of the capture probes described herein). In some embodiments, the capture probes include a capture domain. In some embodiments, the capture domain is substantially complementary to an analyte having a nucleic acid sequence. In some embodiments, the capture domain is substantially complementary to an RNA. In some embodiments, the capture domain is substantially complementary to ribosomal RNA.

In some embodiments, the capture domain includes a sequence that is at least 70%, at least 75%, at least 80%, at least 85%, at least 90%, at least 95%, at least 96%, at least 97%, at least 98%, or at least 99% complementary to a nucleic acid, or a portion thereof. In some embodiments, the capture domain includes a sequence that is perfectly complementary (e.g., is 100% complementary) to a nucleic acid. In some embodiments, the capture domain is capable of capturing nucleic acid from biological samples obtained from different species. For example, rRNA is highly conserved amongst many species and the capture domain can be designed to capture rRNA from biological samples obtained from different species.

In some embodiments of the nucleic acid integrity assay methods described herein, after the capture probe captures an analyte, a reverse transcription reaction is performed thereby generating an extended capture probe (e.g., single stranded cDNA sequence complementary to the captured analyte). Any suitable reverse transcriptase can be used to generate the single-stranded cDNA, including any reverse transcriptases described herein. In some embodiments, extending the end of the capture probe is performed in the presence of actinomycin D.

In some embodiments, the biological sample is treated with a nuclease after the step of extending the capture probe. In some embodiments, the nuclease is an RNase. A non-limiting example of an RNase is RNase H. In some embodiments, the RNase degrades RNA present in the biological sample. In some embodiments, the RNase degrades the captured rRNA hybridized to the extended capture probe (e.g., single-stranded cDNA generated by reverse transcription). In some embodiments after reverse transcription (e.g., single-stranded cDNA synthesis) the biological sample is removed. For example, the biological sample can be treated with one or more permeabilization reagents to remove the biological sample. In some embodiments, the one or more permeabilization reagents include TE buffer and one or more proteases as described herein. In some embodiments, after reverse transcription, the biological sample is not removed.

After treating the biological sample with a nuclease (e.g., RNase) and/or removal of the biological sample, one or more detectable probes can be contacted with the substrate including the capture probes (e.g., array). In some embodiments, the detectable probes are labeled where the detection of the label represents hybridization to the extended capture probe (e.g., single-stranded cDNA), or a complement thereof. The detectable label can be any of the detectable labels described herein (e.g., Cy3, Cy5, etc.). In some embodiments, a first detectable probe is contacted with the array where the first detectable probe hybridizes to a portion of the extended capture probe (e.g., single-stranded cDNA), or a complement thereof. In some embodiments, the first detectable probe is detected by microscope scanning for the fluorophores. In some embodiments, the first detectable probe is disassociated (e.g., dehybridized and washed) from the array. The process of contacting the array with one or more detectable probes (e.g., a first detectable probe, a second detectable probe, a third detectable probe, a fourth detectable probe, or more) followed by disassociation, can be repeated 2, 3, 4, or more times. In some embodiments, one or more second detectable probes are contacted with the array where a detectable probe hybridizes to a portion of the extended capture probe (e.g., single-stranded cDNA), or a complement thereof. In some embodiments, the one or more second detectable probes are detected by microscope scanning for the fluorophores. In some embodiments, the one or more second detectable probes are disassociated (e.g., dehybridized and washed) from the array. In some embodiments, one or more third detectable probes are contacted with the array where a third detectable probe hybridizes to a portion of the extended captured probe (e.g., single-stranded cDNA), or a complement thereof. In some embodiments, the one or more third detectable probes are detected by microscope scanning for the fluorophores. In some embodiments, the one or more third detectable probes are disassociated (e.g., dehybridized and washed) from the array.

In some embodiments of the RNA integrity assay methods described herein, a spatial fragment distribution value (DV) heat map can be generated by detecting a first detectable probe, a second detectable probe, and a third detectable probe. In some embodiments the first, second, and/or third detectable probes can be designed to assess the integrity of the RNA present in a biological sample.

In some embodiments, a detectable probe can be from about 10 nucleotides long to about 30 nucleotides long. In some embodiments, a detectable probe can be from about 15 nucleotides long to about 25 nucleotides long. In some embodiments, a detectable probe can be about 20 nucleotides long. In some embodiments, a detectable probe can be from about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides long.

In some embodiments, the first detectable probe, the second detectable probe, and the third detectable probe include a detectable label (e.g., any of the detectable labels described herein). In some embodiments, the detectable label is a fluorophore. In some embodiments, the first detectable label, the second detectable label, and/or the third detectable label are the same. In some embodiments, the first detectable label, the second detectable label, and/or the third detectable label are different. In some embodiments, the first detectable label, the second detectable label, and/or the third detectable label are detected on the substrate. In some embodiments, the first detectable label, the second detectable label, and/or the third detectable label are compared to generate a spatial fragment DV heat map. In some embodiments, the first detectable label, the second detectable label, and/or the third detectable label are compared to generate a spatial fragment DV number. In some embodiments, the first detectable probe, the second detectable probe, and the third detectable probe are contacted with the substrate sequentially, with disassociation of the previously applied probe as further described herein. In some embodiments, the first detectable probe, the second detectable probe, and the third detectable probe are contacted with the substrate simultaneously.

In some embodiments, detectable probes detect short single-stranded cDNA (e.g., cDNA generated from 18S rRNA), or a complement thereof. In some embodiments, a “short” single-stranded cDNA, or a complement thereof, includes a cDNA about 60 nucleotides or less from the 3′ end of the captured analyte. Thus, for example, a detectable probe designed to detect a short cDNA (e.g., an extended capture probe), or a complement thereof, can be designed to detect a single-stranded cDNA sequence, or complement thereof, between position 1 and position 60 (e.g., short extended capture probe) from the 3′ end of the captured analyte (e.g., 18S rRNA), or complement thereof. In some embodiments, detectable probes detect mid-length extended capture probes (e.g., single stranded cDNA generated from 18S rRNA), or a complement thereof. In some embodiments, a “mid-length” extended capture probe (e.g., single-stranded cDNA) includes cDNA that includes a sequence, or complement thereof, from about 120 nucleotides to about 180 nucleotides from the 3′ end of the captured analyte. Thus, for example, a detectable probe designed to detect a mid-length extended capture probe (e.g., single-stranded cDNA) can be designed to detect a single-stranded cDNA sequence, or complement thereof, between position 120 and position 175 from the 3′ end of the captured analyte (e.g., 18S rRNA). In some embodiments, the one or more second probes detect a mid-length extended capture probe, or complement thereof. In some embodiments, the one or more second detectable probes are positioned 5′ to the location of the first detectable probe. In some embodiments, a “long” extended capture probe (e.g., single-stranded cDNA) includes a sequence, or complement thereof, from about 180 nucleotides to about 220 nucleotides from the 3′ end of the captured analyte. Thus, for example, a detectable probe designed to detect a long extended capture probe (e.g., single-stranded cDNA) can be designed to detect a single-stranded cDNA sequence, or complement thereof, between position 180 and position 220 (or more) from the 3′ end of the captured analyte (e.g., 18S rRNA). In some embodiments, the one or more third detectable probes detect a long extended capture probe, or complement thereof. In some embodiment, the one or more detectable probes are located at a position 5′ to the second detectable probe. For example, an extended capture probe (e.g., single-stranded cDNA), or complement thereof, including a sequence of 100 nucleotides of the captured analyte, or a complement thereof, can be detected by a detectable probe designed to hybridize to the short extended capture probe, but would not be detected by a detectable probe designed to detect a long extended capture probe (e.g., single-stranded cDNA), or a complement thereof. Conversely, an extended capture probe (e.g., a single-stranded cDNA), or a complement thereof, including a sequence of 250 nucleotides of the captured analyte, or a complement thereof, can be detected by a detectable probe designed to hybridize to a short extended capture probe (e.g., single-stranded cDNA), a mid-range extended capture probe, and a long extended capture probe, or complements thereof.

In some embodiments, the first detectable probes are contacted with the biological sample and specifically bind to the extended capture probe (e.g., single-stranded cDNA), or complement thereof, and are detected (e.g., fluorescence is detected from the detectable label) and the image is recorded. In some embodiments, the first detectable probe can be disassociated (e.g., removed) and the process is repeated for a second, a third, or more detectable probes. Thus, for example, the recorded images from each of the detectable probes can be compared to generate a spatial fragment (DV) heat map. In some embodiments, the spatial fragment DV heat map can represent the level of nucleic acid degradation present in the biological sample. In some embodiments, the spatial fragment DV heat map can be represented as one or more spatial fragment DV numbers (e.g., 1 to 100) for the individual detectable probes. For example, a biological sample can have one or more spatial fragment DV numbers that correspond to the location where the one or more detectable probes hybridized to the extended capture probe (e.g., single-stranded cDNA), or complement thereof. For example, a biological sample can have one or more spatial fragment DV numbers that correspond with the contacted first, second, and third detectable probes designed to detect short, mid-range, and long extended capture probes, or complements thereof, respectively.

In some embodiments, a spatial fragment DV number for a long extended capture probe (e.g., single-stranded cDNA), or a complement thereof, is indicative of RNA of sufficient integrity (e.g., lack of degradation) for other downstream analyses, such as spatial transcriptomics, can be from about 30 to about 100, about 40 to about 100, about 50 to about 100, about 60 to about 100, about 70 to about 100, about 80 to about 100, about 90 to about 100, about 30 to about 90, about 40 to about 90, about 50 to about 90, about 60 to about 90, about 70 to about 90, about 80 to about 90, about 30 to about 80, about 40 to about 80, about 50 to about 80, about 60 to about 80, about 70 to about 80, about 30 to about 70, about 40 to about 70, about 50 to about 70, about 60 to about 70, about 30 to about 60, about 40 to about 60, about 50 to about 60, about 30 to about 50, about 40 to about 50, or about 30 to about 40. In some embodiments a spatial fragment DV number for a long single-stranded cDNA (e.g., extended capture probe), or complement thereof, indicative of RNA integrity sufficient for other downstream analyses, such as spatial transcriptomics, can be about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100.

In some embodiments a biological sample can have a low (e.g., less than 3) RNA integrity number or RNA integrity number (RIN) score. High quality RNA is defined as full-length (or close to full-length) transcripts, whereas low quality RNA is defined as fragmented transcripts. RIN values range from 1 to 10, with higher numbers indicating higher quality (e.g., less degraded, less fragmented) RNA samples. In some embodiments, a biological sample can have a spatial fragment DV number for a long single-stranded cDNA less than 30 and a RIN scope of less than 3, where both assays indicate that a biological sample contains degraded RNA of insufficient integrity for other downstream applications. In some embodiments a biological sample can have a low (e.g., less than 3) RIN score and a spatial fragment DV number for a long single-stranded cDNA, or complement thereof, of 30 or above. Thus, for example, an RNA integrity assay, such as a spatial fragment DV assay, can identify biological samples (e.g., fixed biological samples) that may contain RNA of sufficient integrity for further downstream analyses not identified by a RIN score.

### C. Methods and Systems for Analyzing Analytes and Derivatives Thereof

### (a) Introduction

Provided herein are methods and systems for analyzing the location and abundance of a nucleic acid or protein analyte in a fixed biological sample. In some instances, the methods include aligning (i.e., sandwiching) a first substrate having the fixed biological sample with a second substrate that includes a plurality of capture probes, thereby “sandwiching” the fixed biological sample between the two substrates. Upon interaction of the fixed biological sample with the substrate having a plurality of probes (in either instance), the location and abundance of a nucleic acid or protein analyte in a fixed biological sample can be determined, as provided herein. These method include an advantage in that steps provided herein prior to analyte or analyte-derived molecule by the capture probe, most-if not all-steps can be performed on a substrate that does not have capture probes, thereby providing a method that is cost effective.

The methods and systems provided herein can be applied to an analyte or an analyte-derived molecule(s). As used herein, an “analyte derived molecule” includes, without limitation, a connected probe (e.g., a ligation product) from an RNA-templated ligation (RTL) assay, a product of reverse transcription (e.g., an extended capture probe), and an analyte binding moiety barcode (e.g., a binding moiety barcode that identifies that analyte binding moiety (e.g., an antibody)). In some embodiments, the analyte or analyte derived molecules comprise RNA and/or DNA. In some embodiments, the analyte or analyte derived molecules comprise one or more proteins.

In some instances, the methods and systems disclosed herein provide efficient release of an analyte or analyte derived molecule from a biological sample so that it can be easily captured or detected using methods disclosed herein.

In some instances, the methods and systems disclosed herein allow for detection of analytes or analyte derived molecules from different biological samples using a single array including a plurality of capture probes. As such, in some instances, the methods and systems allow for serial capture of analytes or analyte derived molecules from multiple samples. The analytes or analyte derived molecules can then be de-multiplexed using biological-sample-specific index sequences to identify it biological sample origin.

Embodiments of the methods and systems disclosed herein are provided below.

### (A) Exemplary First and Second Substrates

In some instances, the biological sample is placed (e.g., mounted or otherwise immobilized) on a first substrate. The first substrate can be any solid or semi-solid support upon which a biological sample can be mounted. In some instances, the first substrate is a slide. In some instances, the slide is a glass slide. In some embodiments, the substrate is made of glass, silicon, paper, hydrogel, polymer monoliths, or other material known in the art. In some embodiments, the first substrate is comprised of an inert material or matrix (e.g., glass slides) that has been functionalized by, for example, treating the substrate with a material comprising reactive groups which facilitate mounting of the biological sample.

In some embodiments, the first substrate does not comprise a plurality (e.g., array) of capture probes, each comprising a spatial barcode.

A substrate, e.g., a first substrate and/or a second substrate, can generally have any suitable form or format. For example, a substrate can be flat, curved, e.g., convexly or concavely curved. For example, a first substrate can be curved towards the area where the interaction between a biological sample, e.g., tissue sample, and a first substrate takes place. In some embodiments, a substrate is flat, e.g., planar, chip, or slide. A substrate can contain one or more patterned surfaces within the first substrate (e.g., channels, wells, projections, ridges, divots, etc.).

A substrate, e.g., a first substrate and/or second substrate, can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments wherein a substrate structure is flat, the substrate structure can be any appropriate type of support having a flat surface (e.g., a chip or a slide such as a microscope slide).

First and/or second substrates can optionally include various structures such as, but not limited to, projections, ridges, and channels. A substrate can be micropatterned to limit lateral diffusion of analytes (e.g., to improve resolution of the spatial analysis). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a first and/or second substrate is modified to contain one or more wells, using techniques such as (but not limited to) stamping, microetching, or molding techniques. In some embodiments in which a first and/or second substrate includes one or more wells, the first substrate can be a concavity slide or cavity slide. For example, wells can be formed by one or more shallow depressions on the surface of the first and/or second substrate. In some embodiments, where a first and/or second substrate includes one or more wells, the wells can be formed by attaching a cassette (e.g., a cassette containing one or more chambers) to a surface of the first substrate structure.

In some embodiments where the first and/or second substrate is modified to contain one or more structures, including but not limited to, wells, projections, ridges, features, or markings, the structures can include physically altered sites. For example, a first and/or second substrate modified with various structures can include physical properties, including, but not limited to, physical configurations, magnetic or compressive forces, chemically functionalized sites, chemically altered sites, and/or electrostatically altered sites. In some embodiments where the first substrate is modified to contain various structures, including but not limited to wells, projections, ridges, features, or markings, the structures are applied in a pattern. Alternatively, the structures can be randomly distributed.

In some embodiments, a first substrate includes one or more markings on its surface, e.g., to provide guidance for aligning at least a portion of the biological sample with a plurality of capture probes on the second substrate during a sandwich process disclosed herein. For example, the first substrate can include a sample area indicator identifying the sample area. In some embodiments, during a sandwiching process described herein the sample area indicator on the first substrate is aligned with an area of the second substrate comprising a plurality of capture probes. In some embodiments, the first and/or second substrate can include a fiducial mark. In some embodiments, the first and/or second substrate does not comprise a fiducial mark. In some embodiments, the first substrate does not comprise a fiducial mark and the second substrate comprises a fiducial mark. Such markings can be made using techniques including, but not limited to, printing, sand-blasting, and depositing on the surface.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, and colorimetric labels. The use of fiducial markers to stabilize and orient biological samples is described, for example, in Carter et al., Applied Optics 46:421-427, 2007), the entire contents of which are incorporated herein by reference. In some embodiments, a fiducial marker can be a physical particle (e.g., a nanoparticle, a microsphere, a nanosphere, a bead, a post, or any of the other exemplary physical particles described herein or known in the art).

In some embodiments, a fiducial marker can be present on a first substrate to provide orientation of the biological sample. In some embodiments, a microsphere can be coupled to a first substrate to aid in orientation of the biological sample. In some examples, a microsphere coupled to a first substrate can produce an optical signal (e.g., fluorescence). In some embodiments, a quantum dot can be coupled to the first substrate to aid in the orientation of the biological sample. In some examples, a quantum dot coupled to a first substrate can produce an optical signal.

In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Although not required, it can be advantageous to use a marker that can be detected using the same conditions (e.g., imaging conditions) used to detect a labelled cDNA.

In some embodiments, a fiducial marker can be randomly placed in the field of view. For example, an oligonucleotide containing a fluorophore can be randomly printed, stamped, synthesized, or attached to a first substrate (e.g., a glass slide) at a random position on the first substrate. A tissue section can be contacted with the first substrate such that the oligonucleotide containing the fluorophore contacts, or is in proximity to, a cell from the tissue section or a component of the cell (e.g., an mRNA or DNA molecule). An image of the first substrate and the tissue section can be obtained, and the position of the fluorophore within the tissue section image can be determined (e.g., by reviewing an optical image of the tissue section overlaid with the fluorophore detection). In some embodiments, fiducial markers can be precisely placed in the field of view (e.g., at known locations on a first substrate). In this instance, a fiducial marker can be stamped, attached, or synthesized on the first substrate and contacted with a biological sample. Typically, an image of the sample and the fiducial marker is taken, and the position of the fiducial marker on the first substrate can be confirmed by viewing the image.

In some embodiments, a fiducial marker can be an immobilized molecule (e.g., a physical particle) attached to the first substrate. For example, a fiducial marker can be a nanoparticle, e.g., a nanorod, a nanowire, a nanocube, a nanopyramid, or a spherical nanoparticle. In some examples, the nanoparticle can be made of a heavy metal (e.g., gold). In some embodiments, the nanoparticle can be made from diamond. In some embodiments, the fiducial marker can be visible by eye.

A wide variety of different first substrates can be used for the foregoing purposes. In general, a first substrate can be any suitable support material. Exemplary first substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Among the examples of first substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In another example, a first substrate can be a flow cell. Flow cells can be formed of any of the foregoing materials, and can include channels that permit reagents, solvents, features, and analytes to pass through the flow cell. In some embodiments, a hydrogel embedded biological sample is assembled in a flow cell (e.g., the flow cell is utilized to introduce the hydrogel to the biological sample). In some embodiments, a hydrogel embedded biological sample is not assembled in a flow cell. In some embodiments, the hydrogel embedded biological sample can then be prepared and/or isometrically expanded as described herein.

Exemplary substrates similar to the first substrate (e.g., a substrate having no capture probes) and/or the second substrate are described in Section (I) above and in WO 2020/123320, which is hereby incorporated by reference in its entirety.

### (b) Capturing Nucleic Acid Analytes Using RNA-Templated Ligation

In some embodiments, the methods and systems described herein utilize RNA-templated ligation to detect the analyte. As used herein, spatial “RNA-templated ligation,” or “RTL” or simply “templated ligation” is a process wherein individual probe oligonucleotides (e.g., a first probe oligonucleotide, a second probe oligonucleotide) in a probe pair hybridize to adjacent sequences of an analyte (e.g., an RNA molecule) in a biological sample (e.g., a tissue sample). The RTL probe oligonucleotides are then coupled (e.g., ligated) together, thereby creating a connected probe (e.g., a ligation product). RNA-templated ligation is disclosed in PCT Publ. No. WO 2021/133849 A1 and US Publ. No. US 2021/0285046 A1, each of which is incorporated by reference in its entirety.

An advantage to using RTL is that it allows for enhanced detection of analytes (e.g., low expressing analytes) because both probe oligonucleotides must hybridize to the analyte in order for the coupling (e.g., ligating) reaction to occur. As used herein, “coupling” refers to an interaction between two probe oligonucleotides that results in a single connected probe that comprises the two probe oligonucleotides. In some instances, coupling is achieved through ligation. In some instances, coupling is achieved through extension of one probe oligonucleotide to the second probe oligonucleotide followed by ligation. In some instances, coupling is achieved through hybridization (e.g., using a third probe oligonucleotide that hybridized to each of the two probe oligonucleotides) followed by extension of one probe oligonucleotide or gap filling of the sequence between the two probe oligonucleotides using the third probe oligonucleotide as a template.

The connected probe (e.g., ligation product) that results from the coupling (e.g., ligation) of the two probe oligonucleotides can serve as a proxy for the target analyte, as such an analyte derived molecule. Further, it is appreciated that probe oligonucleotide pairs can be designed to cover any gene of interest. For example, a pair of probe oligonucleotides can be designed so that each analyte, e.g., a whole exome, a transcriptome, a genome, can conceivably be detected using a probe oligonucleotide pair.

In some instances, disclosed herein are methods of analyzing an analyte in a fixed biological sample on a first substrate, wherein the fixed biological sample has been affixed to the first substrate for at least four months, the method including (a) hybridizing a first probe and a second probe to the analyte of the fixed biological sample affixed to the first substrate for at least four months, where the first probe and the second probe each include a sequence that is substantially complementary to a first sequence and a second sequence of the analyte, respectively, and where the second probe further includes a capture probe binding domain; (b) coupling the first probe and the second probe, thereby generating a connected probe; (c) aligning the first substrate with a second substrate including an array, such that at least a portion of the biological sample is aligned with at least a portion of the array, where the array includes a plurality of capture probes, where a capture probe of the plurality of capture probes includes: (i) a spatial barcode and (ii) a capture domain; (d) when the biological sample is aligned with at least a portion of the array, (i) releasing the connected probe from the analyte and (ii) migrating the connected probe from the biological sample to the array; and (e) hybridizing the connected probe to the capture domain.

Also provided herein are methods of analyzing an analyte in a fixed biological sample on a first substrate, the method including (a) isolating a portion of the fixed biological sample on the first substrate; (b) determining the presence or absence of RNA of sufficient integrity in the portion of the fixed biological sample; (c) when RNA of sufficient integrity is present in the portion of the fixed biological sample, hybridizing a first probe and a second probe to the analyte, where the first probe and the second probe each include a sequence that is substantially complementary to a first sequence and a second sequence of the analyte, respectively, and where the second probe further includes a capture probe binding domain; (d) coupling the first probe and the second probe, thereby generating a connected probe; (e) aligning the first substrate with a second substrate including an array, such that at least a portion of the biological sample is aligned with at least a portion of the array, where the array includes a plurality of capture probes, where a capture probe of the plurality of capture probes includes: (i) a spatial barcode and (ii) a capture domain; (f) when the biological sample is aligned with at least a portion of the array, (i) releasing the connected probe from the analyte and (ii) migrating the connected probe from the biological sample to the array; and (g) hybridizing the connected probe to the capture domain. In some embodiments, step (c) includes contacting the fixed biological sample with the first probe and the second probe and where upon the contacting the first probe and the second probe hybridize to the analyte. In some embodiments, the process of transferring the connected probe (e.g., a ligation product) from the first substrate to the second substrate is referred to as a “sandwich” process.

The sandwich process is described in PCT Patent Application Publication No. WO 2020/123320, which is incorporated by reference in its entirety. Described herein are methods in which an array with capture probes located on a substrate and a biological sample located on a different substrate, are contacted such that the array is in contact with the biological sample (e.g., the substrates are sandwiched together). In some embodiments, the array and the biological sample can be contacted (e.g., sandwiched), without the aid of a substrate holder. In some embodiments, the array and biological sample substrates can be placed in a substrate holder (e.g., an array alignment device) designed to align the biological sample and the array.

For example, the substrate holder can have placeholders for two substrates. In some embodiments, an array including capture probes can be positioned on one side of the substrate holder (e.g., in a first substrate placeholder). In some embodiments, a biological sample can be placed on the adjacent side of the substrate holder in a second placeholder. In some embodiments, a hinge can be located between the two substrate placeholders that allows the substrate holder to close, e.g., make a sandwich between the two substrate placeholders. In some embodiments, when the substrate holder is closed the biological sample and the array with capture probes are contacted with one another under conditions sufficient to allow analytes present in the biological sample to interact with the capture probes of the array. For example, dried permeabilization reagents can be placed on the biological sample and rehydrated. A permeabilization solution can be flowed through the substrate holder to permeabilize the biological sample and allow analytes in the biological sample to interact with the capture probes. Additionally, the temperature of the substrates or permeabilization solution can be used to initiate or control the rate of permeabilization. For example, the substrate including the array, the substrate including the biological sample, or both substrates can be held at a low temperature to slow diffusion and permeabilization efficiency. Once sandwiched, in some embodiments, the substrates can be heated to initiate permeabilization and/or increase diffusion efficiency. Transcripts that are released from the permeabilized tissue can diffuse to the array and be captured by the capture probes. The sandwich can be opened, and cDNA synthesis can be performed on the array.

In some embodiments, the methods as disclosed herein include hybridizing of one or more probe oligonucleotide probe pairs (e.g., RTL probes) to adjacent or nearby sequences of a target analyte (e.g., RNA; e.g., mRNA) of interest. In some embodiments, the probe oligonucleotide pairs include sequences that are complementary or substantially complementary to an analyte. For example, in some embodiments, each probe oligonucleotide includes a sequence that is complementary or substantially complementary to an mRNA of interest (e.g., to a portion of the sequence of an mRNA of interest). In some embodiments, each target analyte includes a first target region and a second target region. In some embodiments, the methods include providing a plurality of first probe oligonucleotides and a plurality of second probe oligonucleotides, wherein a pair of probe oligonucleotides for a target analyte comprises both a first and second probe oligonucleotide. In some embodiments, a first probe oligonucleotide hybridizes to a first target region of the analyte, and the second probe oligonucleotide hybridizes to a second, adjacent or nearly adjacent target region of the analyte.

In some instances, the probe oligonucleotides are DNA molecules. In some instances, the first probe oligonucleotide is a DNA molecule. In some instances, the second probe oligonucleotide is a DNA molecule. In some instances, the first probe oligonucleotide comprises at least two ribonucleic acid bases at the 3′ end. In some instances, the second probe oligonucleotide comprises a phosphorylated nucleotide at the 5′ end.

RTL probes can be designed using methods known in the art. In some instances, probe pairs are designed to cover an entire transcriptome of a species (e.g., a mouse or a human). In some instances, RTL probes are designed to cover a subset of a transcriptome (e.g., a mouse or a human). In some instances, the methods disclosed herein utilize about 500, about 1000, about 2000, about 3000, about 4000, about 5000, about 6000, about 7000, about 8000, about 9000, about 10,000, about 15,000, about 20,000, or more probe pairs.

In some embodiments, one of the probe oligonucleotides of the pair of probe oligonucleotides for RTL includes a poly(A) sequence or a complement thereof. In some instances, the poly(A) sequence or a complement thereof is on the 5′ end of one of the probe oligonucleotides. In some instances, the poly(A) sequence or a complement thereof is on the 3′ end of one of the probe oligonucleotides. In some embodiments, one probe oligonucleotide of the pair of probe oligonucleotides for RTL includes a degenerate or UMI sequence. In some embodiments, the UMI sequence is specific to a particular target or set of targets. In some instances, the UMI sequence or a complement thereof is on the 5′ end of one of the probe oligonucleotides. In some instances, the UMI sequence or a complement thereof is on the 3′ end of one of the probe oligonucleotides.

In some instances, the first and second target regions of an analyte are directly adjacent to one another. In some embodiments, the complementary sequences to which the first probe oligonucleotide and the second probe oligonucleotide hybridize are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, or about 150 nucleotides away from each other. Gaps between the probe oligonucleotides may first be filled prior to coupling (e.g., ligation), using, for example, dNTPs in combination with a polymerase such as polymerase mu, DNA polymerase, RNA polymerase, reverse transcriptase, VENT polymerase, Taq polymerase, and/or any combinations, derivatives, and variants (e.g., engineered mutants) thereof. In some embodiments, when the first and second probe oligonucleotides are separated from each other by one or more nucleotides, deoxyribonucleotides are used to extend and couple (e.g., ligate) the first and second probe oligonucleotides.

In some instances, the first probe oligonucleotide and the second probe oligonucleotide hybridize to an analyte on the same transcript. In some instances, the first probe oligonucleotide and the second probe oligonucleotide hybridize to an analyte on the same exon. In some instances, the first probe oligonucleotide and the second probe oligonucleotide hybridize to an analyte on different exons. In some instances, the first probe oligonucleotide and the second probe oligonucleotide hybridize to an analyte that is the result of a translocation event (e.g., in the setting of cancer). The methods provided herein make it possible to identify alternative splicing events, translocation events, and mutations that change the hybridization rate of one or both probe oligonucleotides (e.g., single nucleotide polymorphisms, insertions, deletions, point mutations).

In some embodiments, the first and/or second probe as disclosed herein includes at least two ribonucleic acid bases at the 3′ end; a functional sequence; a phosphorylated nucleotide at the 5′ end; and/or a capture probe binding domain. In some embodiments, the functional sequence is a primer sequence.

The “capture probe binding domain” is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or a combination thereof. In some embodiments, the capture probe binding domain includes a random sequence (e.g., a random hexamer or octamer). In some embodiments, the capture probe binding domain is complementary to a capture domain in a capture probe that detects a particular target(s) of interest. In some embodiments, a capture probe binding domain blocking moiety that interacts with the capture probe binding domain is provided. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a connected probe (e.g., a ligation product)) to a capture probe. In some embodiments, a capture probe binding domain blocking moiety comprises a poly-uridine sequence, a poly-thymidine sequence, or a combination thereof.

Hybridization of the probe oligonucleotides to the target analyte can occur at a target having a sequence that is 100% complementary to the probe oligonucleotide(s). In some embodiments, hybridization can occur at a target having a sequence that is at least (e.g. at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe oligonucleotide(s). After hybridization, in some embodiments, the first probe oligonucleotide is extended. After hybridization, in some embodiments, the second probe oligonucleotide is extended. For example, in some instances a first probe oligonucleotide hybridizes to a target sequence upstream for a second oligonucleotide probe, whereas in other instances a first probe oligonucleotide hybridizes to a target sequence downstream of a second probe oligonucleotide.

In some embodiments, methods disclosed herein include a wash step after hybridizing the first and the second probe oligonucleotides. The wash step removes any unbound oligonucleotides and can be performed using any technique known in the art. In some embodiments, a pre-hybridization buffer is used to wash the sample. In some embodiments, a phosphate buffer is used. In some embodiments, multiple wash steps are performed to remove unbound oligonucleotides. For example, it is advantageous to decrease the amount of unhybridized probes present in a biological sample as they may interfere with downstream applications and methods.

In some embodiments, after hybridization of probe oligonucleotides (e.g., first and the second probe oligonucleotides) to the target analyte, the probe oligonucleotides (e.g., the first probe oligonucleotide and the second probe oligonucleotide) are coupled (e.g., ligated) together, creating a single connected probe (e.g., a ligation product) that is complementary to the target analyte. Ligation can be performed enzymatically or chemically, as described herein. For example, the first and second probe oligonucleotides are hybridized to the first and second target regions of the analyte, and the probe oligonucleotides are subjected to a nucleic acid reaction to ligate them together. For example, the probes may be subjected to an enzymatic ligation reaction using a ligase (e.g., T4 RNA ligase (Rnl2), a SplintR ligase, or a T4 DNA ligase). See, e.g., Zhang L., et al.; Archaeal RNA ligase from Thermococcus kodakarensis for template dependent ligation RNA Biol. 2017; 14(1): 36-44 for a description of KOD ligase. A skilled artisan will understand that various reagents, buffers, cofactors, etc. may be included in a ligation reaction depending on the ligase being used.

In some embodiments, the first probe oligonucleotide and the second probe oligonucleotides are on a contiguous nucleic acid sequence. In some embodiments, the first probe oligonucleotide is on the 3′ end of the contiguous nucleic acid sequence. In some embodiments, the first probe oligonucleotide is on the 5′ end of the contiguous nucleic acid sequence. In some embodiments, the second probe oligonucleotide is on the 3′ end of the contiguous nucleic acid sequence. In some embodiments, the second probe oligonucleotide is on the 5′ end of the contiguous nucleic acid sequence.

In some embodiments, the first sequence and the second sequence are adjacent sequences of the analyte. In some embodiments, the first sequence and the second sequence are not adjacent to each other on the analyte. In some embodiments, the method further comprises extending the first probe to generate an extended first probe, thereby filling a gap between the hybridized first probe and the hybridized second probe. In some embodiments, the method further comprises generating an extended second probe using a polymerase, wherein the extended second probe comprises a sequence complementary to a sequence between the sequence hybridized to the first probe and the sequence hybridized to the second probe.

In some embodiments, the method further includes hybridizing a third probe oligonucleotide to the first probe oligonucleotide and the second probe oligonucleotide such that the first probe oligonucleotide and the second probe oligonucleotide abut each other. In some embodiments, the third probe oligonucleotide comprises a sequence that is at least 80%, at least 85%, at least 90%, at least 95%, at least 96%, at least 97%, at least 98%, or at least 99% complementary to a portion of the first probe oligonucleotide that hybridizes to the third probe oligonucleotide. In some embodiments, the third probe oligonucleotide comprises a sequence that is 100% complementary to a portion of the first probe oligonucleotide that hybridizes to the third probe oligonucleotide. In some embodiments, the third probe oligonucleotide comprises a sequence that is at least 80%, at least 85%, at least 90%, at least 95%, at least 96%, at least 97%, at least 98%, or at least 99% complementary to a portion of the second probe oligonucleotide that hybridizes to the third probe oligonucleotide. In some embodiments, the third probe oligonucleotide comprises a sequence that is 100% complementary to a portion of the second probe oligonucleotide that hybridizes to the third probe oligonucleotide.

In some embodiments, a method for identifying a location of an analyte in a biological sample exposed to different permeabilization conditions includes (a) contacting the biological sample with a substrate, wherein the substrate comprises a plurality of capture probes, wherein a capture probe of the plurality of capture probes comprises a capture domain; (b) contacting the biological sample with a first probe oligonucleotide and a second probe oligonucleotide, wherein the first probe oligonucleotide and the second probe oligonucleotide are substantially complementary to adjacent sequences of the analyte, and wherein the second probe oligonucleotide comprises a capture probe-binding domain that is capable of binding to a capture domain of the capture probe; (c) hybridizing the first probe oligonucleotide and the second probe oligonucleotide to adjacent sequences of the analyte; (d) coupling (e.g., ligating) the first probe oligonucleotide and the second probe oligonucleotide, thereby creating a connected probe (e.g., a connected probe (e.g., a ligation product)) that is substantially complementary to the analyte; (e) releasing the connected probe (e.g., a ligation product) from the analyte; (f) hybridizing the capture probe-binding domain of the connected probe (e.g., a ligation product) to the hybridization domain of the capture probe; (g) hybridizing a padlock oligonucleotide to the connected probe (e.g., a ligation product) bound to the capture domain (e.g., such that the padlock oligonucleotide is circularized), wherein the padlock oligonucleotide comprises: (i) a first sequence that is substantially complementary to a first portion of the connected probe (e.g., a ligation product), (ii) a backbone sequence, and (iii) a second sequence that is substantially complementary to a second portion of the connected probe (e.g., a ligation product); and (i) ligating and amplifying the circularized padlock oligonucleotide (e.g., using rolling circle amplification using the circularized padlock oligonucleotide as a template), thereby creating an amplified circularized padlock oligonucleotide, and using the amplified circularized padlock oligonucleotide to identify the location of the analyte in the biological sample.

In some embodiments, the method further includes amplifying the connected probe (e.g., a ligation product) prior to the releasing step. In some embodiments, the entire connected probe (e.g., a ligation product) is amplified. In some embodiments, only part of the connected probe (e.g., a ligation product) is amplified. In some embodiments, amplification is isothermal. In some embodiments, amplification is not isothermal. Amplification can be performed using any of the methods described herein such as, but not limited to, a strand-displacement amplification reaction, a rolling circle amplification reaction, a ligase chain reaction, a transcription-mediated amplification reaction, an isothermal amplification reaction, and/or a 10 loop-mediated amplification reaction. In some embodiments, amplifying the connected probe (e.g., a ligation product) creates an amplified connected probe (e.g., a ligation product) that includes (i) all or part of sequence of the connected probe (e.g., a ligation product) specifically bound to the capture domain, or a complement thereof, and (ii) all or a part of the sequence of the spatial barcode, or a complement thereof.

In some embodiments, the method further includes determining (i) all or a part of the sequence of the connected probe (e.g., a ligation product), or a complement thereof, and (ii) the sequence of the spatial barcode, or a complement thereof. In some embodiments, the method further includes using the determined sequence of (i) and (ii) to determine the location and abundance of the analyte in the biological sample.

In some embodiments, after coupling (e.g., ligation) of the first and second probe oligonucleotides to create a ligation product, the connected probe (e.g., a ligation product) is released from the analyte. To release the connected probe (e.g., a ligation product), an endoribonuclease (e.g., RNase A, RNase C, RNase H, or RNase I) is used. An endoribonuclease such as RNase H specifically cleaves RNA in RNA:DNA hybrids. In some embodiments, the connected probe (e.g., a ligation product) is released enzymatically. In some embodiments, an endoribonuclease is used to release the probe from the analyte. In some embodiments, the endoribonuclease is one or more of RNase H. In some embodiments, the RNase H is RNase H1 or RNase H2.

In some embodiments, the releasing of the connected probe (e.g., a ligation product) includes contacting the biological sample with a reagent medium comprising a permeabilization agent and an agent for releasing the connected probe (e.g., a ligation product), thereby permeabilizing the biological sample and releasing the connected probe (e.g., a ligation product) from the analyte. In some embodiments, the agent for releasing the connected probe (e.g., a ligation product) comprises a nuclease. In some embodiments, the nuclease is an endonuclease. In some embodiments, the nuclease is an exonuclease. In some embodiments, the nuclease includes an RNase. In some embodiments, the RNase is selected from RNase A, RNase C, RNase H, or RNase I.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In some embodiments, the reagent medium includes a wetting agent.

In some instances, after creation of the connected probe (e.g., a ligation product), the methods disclosed herein include simultaneous treatment of the biological sample with a permeabilization agent such as proteinase K (to permeabilize the biological sample) and a releasing agent such as an endonuclease such as RNase H (to release the connected probe (e.g., a ligation product) from the analyte). In some instances, the permeabilization step and releasing step occur at the same time. In some instances, the permeabilization step occurs before the releasing step. In some embodiments, the permeabilization agent comprises a protease. In some embodiments, the protease is selected from trypsin, pepsin, elastase, or Proteinase K. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin.

In some embodiments, the reagent medium further includes a detergent. In some embodiments, the detergent is selected from sodium dodecyl sulfate (SDS), sarkosyl, saponin, Triton X-100™, or Tween-20™. In some embodiments, the reagent medium includes less than 5 w/v % of a detergent selected from sodium dodecyl sulfate (SDS) and sarkosyl. In some embodiments, the reagent medium includes as least 5% w/v % of a detergent selected from SDS and sarkosyl. In some embodiments, the reagent medium does not include SDS or sarkosyl.

In some embodiments, the biological sample and the array are contacted with the reagent medium for about 1 to about 60 minutes (e.g., about 1 to about 55 minutes, about 1 to about 50 minutes, about 1 to about 45 minutes, about 1 to about 40 minutes, about 1 to about 35 minutes, about 1 to about 30 minutes, about 1 to about 25 minutes, about 1 to about 20 minutes, about 1 to about 15 minutes, about 1 to about 10 minutes, about 1 to about 5 minutes, about 5 to about 60 minutes, about 5 to about 55 minutes, about 5 to about 50 minutes, about 5 to about 45 minutes, about 5 to about 40 minutes, about 5 to about 35 minutes, about 5 to about 30 minutes, about 5 to about 25 minutes, about 5 to about 20 minutes, about 5 to about 15 minutes, about 5 to about 10 minutes, about 10 to about 60 minutes, about 10 to about 55 minutes, about 10 to about 50 minutes, about 10 to about 45 minutes, about 10 to about 40 minutes, about 10 to about 35 minutes, about 10 to about 30 minutes, about 10 to about 25 minutes, about 10 to about 20 minutes, about 10 to about 15 minutes, about 15 to about 60 minutes, about 15 to about 55 minutes, about 15 to about 50 minutes, about 15 to about 45 minutes, about 15 to about 40 minutes, about 15 to about 35 minutes, about 15 to about 30 minutes, about 15 to about 25 minutes, about 15 to about 20 minutes, about 20 to about 60 minutes, about 20 to about 55 minutes, about 20 to about 50 minutes, about 20 to about 45 minutes, about 20 to about 40 minutes, about 20 to about 35 minutes, about 20 to about 30 minutes, about 20 to about 25 minutes, about 25 to about 60 minutes, about 25 to about 55 minutes, about 25 to about 50 minutes, about 25 to about 45 minutes, about 25 to about 40 minutes, about 25 to about 35 minutes, about 25 to about 30 minutes, about 30 to about 60 minutes, about 30 to about 55 minutes, about 30 to about 50 minutes, about 30 to about 45 minutes, about 30 to about 40 minutes, about 30 to about 35 minutes, about 35 to about 60 minutes, about 35 to about 55 minutes, about 35 to about 50 minutes, about 35 to about 45 minutes, about 35 to about 40 minutes, about 40 to about 60 minutes, about 40 to about 55 minutes, about 40 to about 50 minutes, about 40 to about 45 minutes, about 45 to about 60 minutes, about 45 to about 55 minutes, about 45 to about 50 minutes, about 50 to about 60 minutes, about 50 to about 55 minutes, or about 55 to about 60 minutes). In some embodiments, the biological sample and the array are contacted with the reagent medium for about 30 minutes.

In some embodiments, the connected probe (e.g., a ligation product) includes a capture probe binding domain, which can hybridize to a capture probe (e.g., a capture probe immobilized, directly or indirectly, on a substrate). In some embodiments, the capture probe includes a spatial barcode and the capture domain. In some embodiments, the capture probe binding domain of the connected probe (e.g., a ligation product) specifically binds to the capture domain of the capture probe.

In some embodiments, methods provided herein include mounting a biological sample on a first substrate, then aligning the first substrate with a second substrate including an array, such that at least a portion of the biological sample is aligned with at least a portion of the array, wherein the array includes a plurality of capture probes. After hybridization of the connected probe (e.g., a ligation product) to the capture probe, downstream methods as disclosed herein can be performed.

In some embodiments, at least 50% of connected probes (e.g., a ligation products) released from the portion of the biological sample aligned with the portion of the array are captured by capture probes of the portion of the array. In some embodiments, at least 50%, at least 55%, at least 60%, at least 65%, at least 70%, at least 75%, at least 85%, at least 90%, at least 95%, at least 96%, at least 97%, at least 98%, at least 99%, or 100% of connected probe (e.g., a ligation products) are detected in spots directly under the biological sample.

In some embodiments, the capture probe includes a poly(T) sequence. In some embodiments, capture probe includes a sequence specific to the analyte. In some embodiments, the capture probe includes a functional domain. In some embodiments, the capture probe further includes one or more functional domains, a unique molecular identifier (UMI), a cleavage domain, and combinations thereof. In some embodiments, the capture probe binding domain includes a poly(A) sequence. In some embodiments, the capture probe binding domain includes a sequence complementary to a capture domain of a capture probe that detects a target analyte of interest. In some embodiments, the analyte is RNA. In some embodiments, the analyte is mRNA.

In some embodiments, the connected probe (e.g., a ligation product) (e.g., the analyte derived molecule) includes a capture probe binding domain, which can hybridize to a capture probe (e.g., a capture probe immobilized, directly or indirectly, on a substrate). Methods provided herein include contacting a biological sample with a substrate, wherein the capture probe is affixed to the substrate (e.g., immobilized to the substrate, directly or indirectly). After hybridization of the connected probe (e.g., a ligation product) to the capture probe, downstream methods as disclosed herein (e.g., sequencing, in situ analysis such as RCA) can be performed.

In some embodiments, the method further includes analyzing a different analyte in the biological sample. In some embodiments, the analysis of the different analyte includes (a) further contacting the biological sample with a plurality of analyte capture agents, wherein an analyte capture agent of the plurality of analyte capture agents includes an analyte binding moiety and a capture agent barcode domain, wherein the analyte binding moiety specifically binds to the different analyte, and wherein the capture agent barcode domain includes an analyte binding moiety barcode and a capture handle sequence that is complementary to a capture domain of a capture probe; and (b) hybridizing the analyte capture sequence to the capture domain.

In some embodiments, the method further includes determining (i) all or part of the sequence of the capture agent barcode domain; and (ii) all or a part of the sequence of the spatial barcode, or a complement thereof. In some embodiments, the method further includes using the determined sequence of (i), and (ii) to analyze the different analyte in the biological sample. In some embodiments, the releasing step further releases the capture agent barcode domain from the different analyte. In some embodiments, the different analyte is a protein analyte. In some embodiments, the protein analyte is an extracellular protein. In some embodiments, the protein analyte is an intracellular protein.

### (c) Capturing Analytes for Spatial Detection Using Analyte Capture Agents

In some embodiments, the methods, compositions, devices, and systems provided herein utilize analyte capture agents for spatial detection. An “analyte capture agent” refers to a molecule that interacts with a target analyte (e.g., a protein) and with a capture probe. Such analyte capture agents can be used to identify the analyte. In some embodiments, the analyte capture agent can include an analyte binding moiety and a capture agent barcode domain. In some embodiments, the analyte capture agent includes a linker. In some embodiments, the linker is a cleavable linker. In some embodiments, the cleavable linker is a photo-cleavable linker, a UV-cleavable linker, or an enzyme cleavable linker.

An analyte binding moiety is a molecule capable of binding to a specific analyte. In some embodiments, the analyte binding moiety comprises an antibody or antibody fragment.

In some embodiments, the analyte binding moiety comprises a polypeptide and/or an aptamer. In some embodiments, the analyte is a protein (e.g., a protein on a surface of a cell or an intracellular protein).

A capture agent barcode domain can include a capture handle sequence which can hybridize to at least a portion or an entirety of a capture domain of a capture probe. In some embodiments, the capture handle sequence is complementary to a portion or entirety of a capture domain of a capture probe. In some embodiments, the capture handle sequence includes a poly (A) tail. In some embodiments, the capture handle sequence includes a sequence capable of binding a poly (T) domain. In some embodiments, the capture agent barcode domain comprises an analyte binding moiety barcode and a capture handle sequence. The analyte binding moiety barcode refers to a barcode that is associated with or otherwise identifies the analyte binding moiety, and the capture handle sequence can hybridize to a capture probe. In some embodiments, the capture handle sequence specifically binds to the capture domain of the capture probe. Other embodiments of an analyte capture agent useful in spatial analyte detection are described herein.

In some embodiments, the process of transferring the connected probe (e.g., a ligation product) from the first substrate to the second substrate is referred to as a “sandwich process”. The sandwich process is described above and in PCT Patent Application Publication No. WO 2020/123320, which is incorporated by reference in its entirety.

In some embodiments, an analyte capture agent is introduced to a biological sample, wherein the analyte binding moiety specifically binds to a target analyte, and then the biological sample can be treated to release the capture agent barcode domain from the biological sample. In some embodiments, the capture agent barcode domain can then migrate and bind to a capture domain of a capture probe, and the capture agent barcode domain can be extended to generate a spatial barcode complement at the end of the capture agent barcode domain. In some embodiments, the spatially-tagged capture agent barcode domain can be denatured from the capture probe, and analyzed using methods described herein.

In some embodiments, the releasing includes contacting the biological sample and the array with a reagent medium including a nuclease. In some embodiments, the nuclease includes an RNase. In some embodiments, the RNase is selected from RNase A, RNase C, RNase H, and RNase I. In some embodiments, the reagent medium further includes a permeabilization agent. In some embodiments, the releasing further includes simultaneously permeabilizing the biological sample and releasing the capture agent barcode domain from the analyte. In some embodiments, the permeabilization agent further includes a protease. In some embodiments, the protease is selected from trypsin, pepsin, elastase, or Proteinase K.

In some embodiments, the capture agent barcode domain is released from the analyte binding moiety by using a different stimulus that can include, but is not limited to, a proteinase (e.g., Proteinase K), an RNase, and UV light.

In some embodiments, the reagent medium further includes a detergent. In some embodiments, the detergent is selected from sodium dodecyl sulfate (SDS), sarkosyl, saponin, Triton X-100™, or Tween-20™. In some embodiments, the reagent medium includes less than 5 w/v % of a detergent selected from sodium dodecyl sulfate (SDS) and sarkosyl. In some embodiments, the reagent medium includes as least 5% w/v % of a detergent selected from SDS and sarkosyl. In some embodiments, the reagent medium does not include SDS or sarkosyl.

In some embodiments, the biological sample and the array are contacted with the reagent medium for about 1 to about 60 minutes (e.g., about 1 to about 55 minutes, about 1 to about 50 minutes, about 1 to about 45 minutes, about 1 to about 40 minutes, about 1 to about 35 minutes, about 1 to about 30 minutes, about 1 to about 25 minutes, about 1 to about 20 minutes, about 1 to about 15 minutes, about 1 to about 10 minutes, about 1 to about 5 minutes, about 5 to about 60 minutes, about 5 to about 55 minutes, about 5 to about 50 minutes, about 5 to about 45 minutes, about 5 to about 40 minutes, about 5 to about 35 minutes, about 5 to about 30 minutes, about 5 to about 25 minutes, about 5 to about 20 minutes, about 5 to about 15 minutes, about 5 to about 10 minutes, about 10 to about 60 minutes, about 10 to about 55 minutes, about 10 to about 50 minutes, about 10 to about 45 minutes, about 10 to about 40 minutes, about 10 to about 35 minutes, about 10 to about 30 minutes, about 10 to about 25 minutes, about 10 to about 20 minutes, about 10 to about 15 minutes, about 15 to about 60 minutes, about 15 to about 55 minutes, about 15 to about 50 minutes, about 15 to about 45 minutes, about 15 to about 40 minutes, about 15 to about 35 minutes, about 15 to about 30 minutes, about 15 to about 25 minutes, about 15 to about 20 minutes, about 20 to about 60 minutes, about 20 to about 55 minutes, about 20 to about 50 minutes, about 20 to about 45 minutes, about 20 to about 40 minutes, about 20 to about 35 minutes, about 20 to about 30 minutes, about 20 to about 25 minutes, about 25 to about 60 minutes, about 25 to about 55 minutes, about 25 to about 50 minutes, about 25 to about 45 minutes, about 25 to about 40 minutes, about 25 to about 35 minutes, about 25 to about 30 minutes, about 30 to about 60 minutes, about 30 to about 55 minutes, about 30 to about 50 minutes, about 30 to about 45 minutes, about 30 to about 40 minutes, about 30 to about 35 minutes, about 35 to about 60 minutes, about 35 to about 55 minutes, about 35 to about 50 minutes, about 35 to about 45 minutes, about 35 to about 40 minutes, about 40 to about 60 minutes, about 40 to about 55 minutes, about 40 to about 50 minutes, about 40 to about 45 minutes, about 45 to about 60 minutes, about 45 to about 55 minutes, about 45 to about 50 minutes, about 50 to about 60 minutes, about 50 to about 55 minutes, or about 55 to about 60 minutes). In some embodiments, the biological sample and the array are contacted with the reagent medium for about 30 minutes.

Also provided herein are methods further including analyzing a different analyte in the biological sample. In some embodiments, the analysis of the different analyte includes (a) hybridizing a first probe oligonucleotide and a second probe oligonucleotide to the different analyte, wherein the first probe oligonucleotide and the second probe oligonucleotide each comprise a sequence that is substantially complementary to adjacent sequences of the different analyte, and wherein the second probe oligonucleotide comprises a capture probe binding domain; (b) ligating the first probe oligonucleotide and the second probe oligonucleotide, thereby generating a connected probe (e.g., a ligation product) comprising the capture probe binding domain; and (c) hybridizing the capture probe binding domain of the connected probe (e.g., a ligation product) to the capture domain.

In some embodiments, the method further includes determining (i) all or part of the sequence of the connected probe (e.g., a ligation product), or a complement thereof, and (ii) all or a part of the sequence of the spatial barcode, or a complement thereof. In some embodiments, the method further includes using the determined sequence of (i), and (ii) to analyze the different analyte in the biological sample. In some embodiments, the releasing step further releases the connected probe (e.g., a ligation product) from the different analyte.

In some embodiments, the different analyte is RNA. In some embodiments, the different analyte is mRNA.

In some embodiments, the capture probe comprises a poly(T) sequence. In some embodiments, the capture probe comprises a sequence complementary to the capture handle sequence. In some embodiments, the capture probe comprises a functional domain. In some embodiments, the capture probe further comprises one or more functional domains, a unique molecular identifier (UMI), a cleavage domain, and combinations thereof.

In some embodiments, the biological sample is a tissue sample. In some embodiments, the tissue sample is a tissue section. In some embodiments, the tissue sample is a fixed tissue sample. In some embodiments, the fixed tissue sample is a formalin fixed paraffin embedded (FFPE) tissue sample. In some embodiments, the FFPE tissue is deparaffinized and decrosslinked prior to step (a) of any one of the methods provided herein. In some embodiments, the fixed tissue sample is a formalin fixed paraffin embedded cell pellet. In some embodiments, the tissue sample is a fresh tissue sample or a frozen tissue sample. In some embodiments, the tissue sample is fixed and stained prior to step (a) of any one of the methods provided herein.

In some instances, RTL is performed between two oligonucleotides that each are affixed to an analyte binding moiety (i.e., a protein-binding moiety). Generally, the methods of RTL in this setting is as follows. In some embodiments, provided herein is a method of determining a location of at least one analyte in a biological sample including: (a) hybridizing a first analyte-binding moiety to a first analyte in the biological sample, wherein the first analyte-binding moiety is bound to a first oligonucleotide, wherein the first oligonucleotide comprises: (i) a functional sequence; (ii) a first barcode; and (iii) a first bridge sequence; (b) hybridizing a second analyte-binding moiety to a second analyte in the biological sample, wherein the second analyte-binding moiety is bound to a second oligonucleotide; wherein the second oligonucleotide comprises: (i) capture probe binding domain sequence, (ii) a second barcode; and (ii) a second bridge sequence; (c) contacting the biological sample with a third oligonucleotide; (d) hybridizing the third oligonucleotide to the first bridge sequence of the first oligonucleotide and second bridge sequence of the second oligonucleotide; (e) ligating the first oligonucleotide and the second oligonucleotide, creating a connected probe (e.g., a ligation product); (f) contacting the biological sample with a substrate, wherein a capture probe is affixed to the substrate, wherein the capture probe comprises a spatial barcode and the capture domain; and (g) allowing the capture probe binding domain sequence of the second oligonucleotide to specifically bind to the capture domain. In some instances, the connected probe (e.g., a ligation product) is cleaved from the analyte biding moieties.

In some instances, two analytes (e.g., two different proteins) in close proximity in a biological sample are detected by a first analyte-binding moiety and a second analyte-binding moiety, respectively. In some embodiments, a first analyte-binding moiety and/or the second analyte-binding moiety is an analyte capture agent (e.g., any of the exemplary analyte capture agents described herein). In some embodiments, the first analyte-binding moiety and/or the second analyte-binding moiety is a first protein. In some embodiments, the first analyte-binding moiety and/or the second analyte-binding moiety is an antibody. For example, the antibody can include, without limitation, a monoclonal antibody, recombinant antibody, synthetic antibody, a single domain antibody, a single-chain variable fragment (scFv), and or an antigen-binding fragment (Fab). In some embodiments, the first analyte-binding moiety binds to a cell surface analyte (e.g., any of the exemplary cell surface analytes described herein). In some embodiments, binding of the analyte is performed metabolically. In some embodiments, binding of the analyte is performed enzymatically. In some embodiments, the methods include a secondary antibody that binds to a primary antibody, enhancing its detection.

In some embodiments, the first analyte-binding moiety and the second analyte-binding moiety each bind to the same analyte. In some embodiments, the first analyte-binding moiety and/or second analyte-binding moiety each bind to a different analyte. For example, in some embodiments, the first analyte-binding moiety binds to a first polypeptide and the second analyte-binding moiety binds to a second polypeptide.

In some embodiments of any of the methods of determining a location of at least one analyte in a biological sample, a first and/or a second oligonucleotide are bound (e.g., conjugated or otherwise attached using any of the methods described herein) to a first analyte-binding moiety and/or a second analyte-binding moiety, respectively.

In some embodiments of any of the methods of determining a location of at least one analyte in a biological sample as described herein, a second oligonucleotide is bound (e.g., conjugated or otherwise attached using any of the methods described herein) to a second analyte-binding moiety. For example, the second oligonucleotide can be covalently linked to the second analyte-binding moiety. In some embodiments, the second oligonucleotide is bound to the second analyte-binding moiety via its 5′ end. In some embodiments, the second oligonucleotide includes a free 3′ end. In some embodiments the second oligonucleotide is bound to the second analyte-binding moiety via its 3′ end. In some embodiments, the second oligonucleotide includes a free 5′ end.

In some embodiments, the oligonucleotides are bound to the first and/or second analyte-binding moieties via a linker (e.g., any of the exemplary linkers described herein). In some embodiments, the linker is a cleavable linker. In some embodiment, the linker is a linker with photo-sensitive chemical bonds (e.g., photo-cleavable linkers). In some embodiments, the linker is a cleavable linker that can undergo induced dissociation.

In some embodiments, the oligonucleotides are bound (e.g., attached via any of the methods described herein) to an analyte-binding domain via a 5′ end.

In some embodiments, a barcode is used to identify the analyte-binding moiety to which it is bound. The barcode can be any of the exemplary barcodes described herein. In some embodiments, the first and/or second oligonucleotide include a capture probe binding domain sequence. For example, a capture probe binding domain sequence can be a poly(A) sequence when the capture domain sequence is a poly(T) sequence.

In some embodiments, a third oligonucleotide (e.g., a splint oligonucleotide) hybridizes to both the first and second oligonucleotides and enables ligation of the first oligonucleotide and the second oligonucleotide. In some embodiments, a ligase is used. In some aspects, the ligase includes a DNA ligase. In some aspects, the ligase includes a RNA ligase. In some aspects, the ligase includes T4 DNA ligase. In some embodiments, the ligase is a SplintR ligase.

### (d) Sandwich Processes

In some embodiments of a method disclosed herein, wherein samples of various ages are examined, one or more analytes from the biological sample are released from the biological sample and migrate to a substrate comprising an array of capture probes for attachment to the capture probes of the array. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., US. Patent Application Pub. No. 20210189475, WO 2021252747A1, and WO 2022061152. In some embodiments, the sandwiching process may be facilitated by a device, sample holder, sample handling apparatus, or system described in, e.g., US. Patent Application Pub. No. 20210189475, WO 2021252747A1, or WO 2022061152.

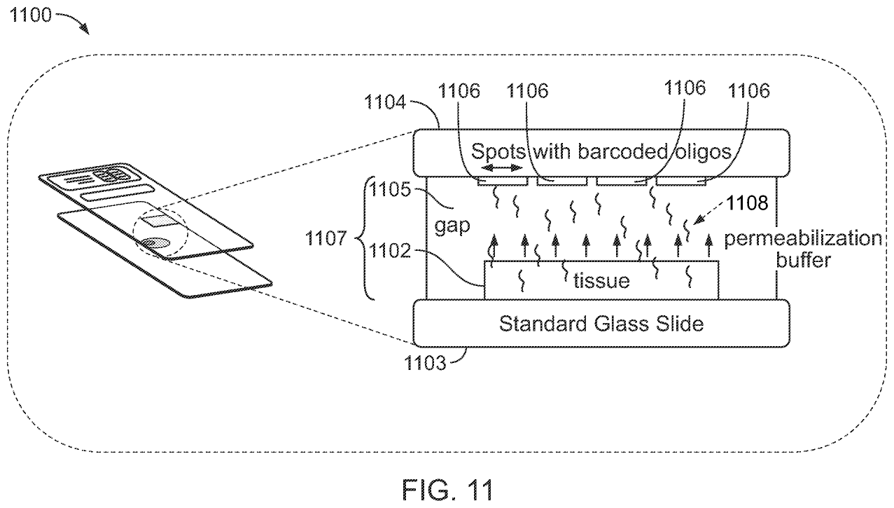

FIG. 11 is a schematic diagram depicting an exemplary sandwiching process 1104 between a first substrate comprising a biological sample (e.g., a tissue section 1102 on a slide 1103) and a second substrate comprising a spatially barcoded array, e.g., a slide 1104 that is populated with spatially-barcoded capture probes 1106. During the exemplary sandwiching process, the first substrate is aligned with the second substrate, such that at least a portion of the biological sample is aligned with at least a portion of the array (e.g., aligned in a sandwich configuration). As shown, the second substrate (e.g., slide 1104) is in a superior position to the first substrate (e.g., slide 1103). In some embodiments, the first substrate (e.g., slide 1103) may be positioned superior to the second substrate (e.g., slide 1104). A reagent medium 1105 (e.g., permeabilization solution) within a gap 1107 between the first substrate (e.g., slide 1103) and the second substrate (e.g., slide 1104) creates a permeabilization buffer which permeabilizes or digests the sample 1102 and the analyte derived molecules (e.g., ligated/connected probes) 1108 of the biological sample 1102 may release, actively or passively migrate (e.g., diffuse) across the gap 1107 toward the capture probes 1106, and bind on the capture probes 1106.

After the analytes (e.g., transcripts) 1108 bind the capture probes 1106, an extension reaction may occur, thereby generating a spatially barcoded library. For example, in the case of mRNA transcripts, reverse transcription may be used to generate a cDNA library associated with a particular spatial barcode. Barcoded cDNA libraries may be mapped back to a specific spot on a capture area of the capture probes 1106. This data may be subsequently layered over a high-resolution microscope image of the biological sample, making it possible to visualize the data within the morphology of the tissue in a spatially-resolved manner. In some embodiments, the extension reaction can be performed separately from the sample handling apparatus described herein that is configured to perform the exemplary sandwiching process 1104. The sandwich configuration of the sample 1102, the first substrate (e.g., slide 1103) and the second substrate (e.g., slide 1104) may provide advantages over other methods of spatial analysis and/or analyte capture. For example, the sandwich configuration may reduce a burden of users to develop in house tissue sectioning and/or tissue mounting expertise. Further, the sandwich configuration may decouple sample preparation/tissue imaging from the barcoded array (e.g., spatially-barcoded capture probes 1106) and enable selection of a particular region of interest of analysis (e.g., for a tissue section larger than the barcoded array). The sandwich configuration also beneficially enables spatial analysis without having to place a biological sample (e.g., tissue section) 1102 directly on the second substrate (e.g., slide 1104).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the analyte from the biological sample.

The sandwiching process methods described above can be implemented using a variety of hardware components. For example, the sandwiching process methods can be implemented using a sample holder (also referred to herein as a support device, a sample handling apparatus, and an array alignment device).

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

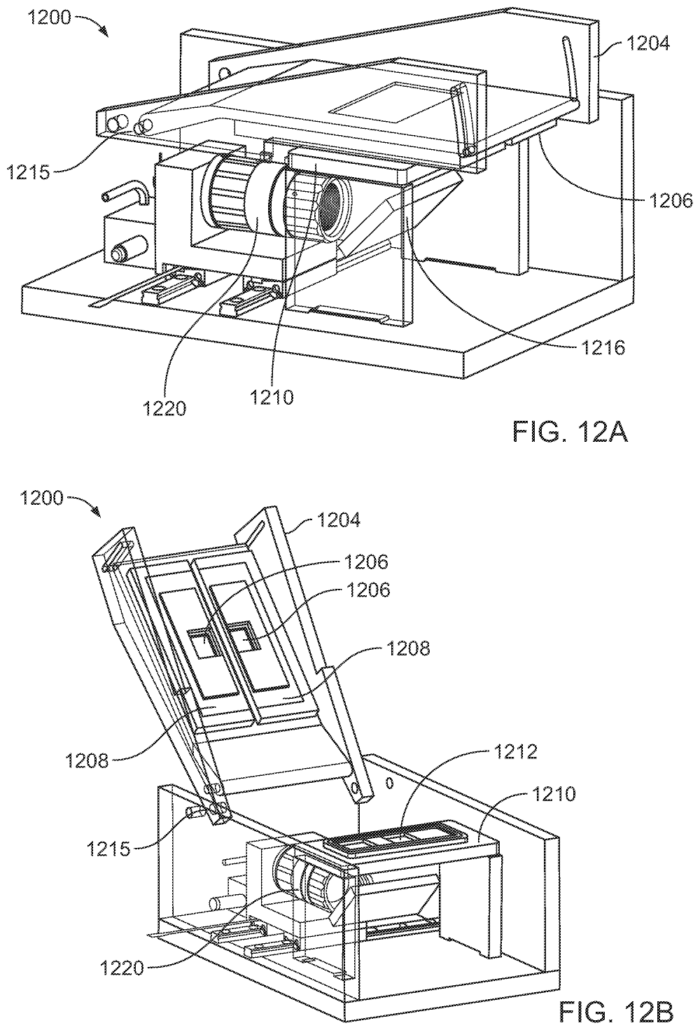

FIG. 12A is a perspective view of an example sample handling apparatus 1200 in a closed position in accordance with some example implementations. As shown, the sample handling apparatus 1200 includes a first member 1204, a second member 1210, optionally an image capture device 1220, a first substrate 1206, optionally a hinge 1215, and optionally a mirror 1216. The hinge 1215 may be configured to allow the first member 1204 to be positioned in an open or closed configuration by opening and/or closing the first member 1204 in a clamshell manner along the hinge 1215.

FIG. 12B is a perspective view of the example sample handling apparatus 1200 in an open position in accordance with some example implementations. As shown, the sample handling apparatus 1200 includes one or more first retaining mechanisms 1208 configured to retain one or more first substrates 1206. In the example of FIG. 12B, the first member 1204 is configured to retain two first substrates 1206, however the first member 1204 may be configured to retain more or fewer first substrates 1206.

In some aspects, when the sample handling apparatus 1200 is in an open position (as in FIG. 12B), the first substrate 1206 and/or the second substrate 1212 may be loaded and positioned within the sample handling apparatus 1200 such as within the first member 1204 and the second member 1210, respectively. As noted, the hinge 1215 may allow the first member 1204 to close over the second member 1210 and form a sandwich configuration (e.g., the sandwich configuration shown in FIG. 11).

In some aspects, after the first member 1204 closes over the second member 1210, an adjustment mechanism (not shown) of the sample handling apparatus 1200 may actuate the first member 1204 and/or the second member 1210 to form the sandwich configuration for the permeabilization step (e.g., bringing the first substrate 1206 and the second substrate 1212 closer to each other and within a threshold distance for the sandwich configuration). The adjustment mechanism may be configured to control a speed, an angle, a force, or the like of the sandwich configuration.

In some embodiments, the biological sample (e.g., sample 1102) may be aligned within the first member 1204 (e.g., via the first retaining mechanism 1208) prior to closing the first member 1204 such that a desired region of interest of the sample 1102 is aligned with the barcoded array of the second substrate (e.g., the slide 1104), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 1206 and/or the second substrate 1212 to maintain a minimum spacing between the first substrate 1206 and the second substrate 1212 during sandwiching. In some aspects, the permeabilization solution (e.g., permeabilization solution 1105) may be applied to the first substrate 1206 and/or the second substrate 1212. The first member 1204 may then close over the second member 1210 and form the sandwich configuration. Analytes derivatives (e.g., connected probes) 1108 may be captured by the capture probes 1106 and may be processed for spatial analysis.

In some embodiments, during the permeabilization step, the image capture device 1220 may capture images of the overlap area between the tissue 1102 and the capture probes 1106. If more than one first substrates 1206 and/or second substrates 1212 are present within the sample handling apparatus 1200, the image capture device 1220 may be configured to capture one or more images of one or more overlap areas. Further details on support devices, sample holders, sample handling apparatuses, or systems for implementing a sandwiching process are described in, e.g., US. Patent Application Pub. No. 20210189475, and WO 2022/061152, each of which are incorporated by reference in their entirety.

Analytes within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein)

Provided herein are methods for delivering a fluid to a biological sample disposed on an area of a first substrate and an array disposed on a second substrate.

In some embodiments and with reference to FIG. 11, the sandwich configuration described herein between a first substrate comprising a biological sample (e.g., slide 1103) and a second substrate comprising a spatially barcoded array (e.g., slide 1104 with barcoded capture probes 1106) may include a reagent medium (e.g., a liquid reagent medium, e.g., a permeabilization solution 1105 or other target molecule release and capture solution) to fill a gap (e.g., gap 1107). It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates (e.g., slide 1103 and slide 1104) during a permeabilization step (e.g., step 1104).

In some aspects, it may be possible to reduce or eliminate bubble formation between the slides using a variety of filling methods and/or closing methods.

Workflows described herein may include contacting a drop of the reagent medium (e.g., liquid reagent medium, e.g., a permeabilization solution 1105) disposed on a first substrate or a second substrate with at least a portion of the second substrate or first substrate, respectively. In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In the example sandwich maker workflows described herein, the reagent medium (e.g., liquid reagent medium, permeabilization solution 1105) may fill a gap (e.g., the gap 1107) between a first substrate (e.g., slide 1103) and a second substrate (e.g., slide 1104 with barcoded capture probes 1106) to warrant or enable transfer of target molecules with spatial information. Described herein are examples of filling methods that may suppress bubble formation and suppress undesirable flow of transcripts and/or target molecules or analytes. Robust fluidics in the sandwich making described herein may preserve spatial information by reducing or preventing deflection of molecules as they move from the tissue slide to the capture slide.

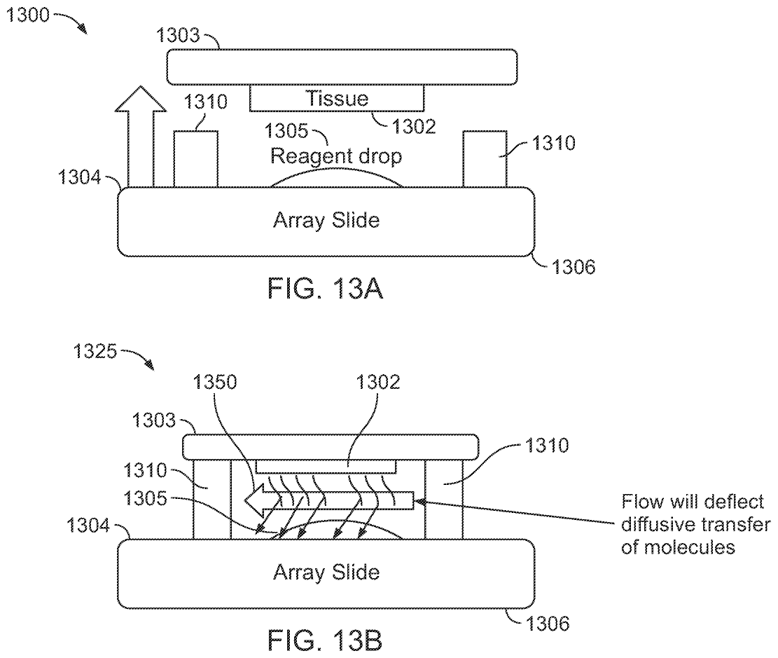

FIG. 13A shows an exemplary sandwiching process 1300 where a first substrate (e.g., slide 1103), including a biological sample 1302 (e.g., a tissue section), and a second substrate (e.g., slide 1306 including spatially barcoded capture probes) are brought into proximity with one another. As shown in FIG. 13A a liquid reagent drop (e.g., permeabilization solution 1305) is introduced on the second substrate in proximity to the capture probes and in between the biological sample 1302 and the second substrate (e.g., slide 1306 including spatially barcoded capture probes). The permeabilization solution 1305 may release analytes that can be captured by the capture probes of the array. As further shown, one or more spacers 1310 may be positioned between the first substrate (e.g., slide 1303) and the second substrate (e.g., slide 1304 including spatially barcoded capture probes). The one or more spacers 1310 may be configured to maintain a separation distance between the first substrate and the second substrate. While the one or more spacers 1310 is shown as disposed on the second substrate, the spacer may additionally or alternatively be disposed on the first substrate.

In some embodiments, the one or more spacers 1310 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 13B shows a fully formed sandwich configuration creating a chamber 1350 formed from the one or more spacers 1310, the first substrate (e.g., the slide 1303), and the second substrate (e.g., the slide 1306 including spatially barcoded capture probes) in accordance with some example implementations. In the example of FIG. 13B, the liquid reagent (e.g., the permeabilization solution 1305) fills the volume of the chamber 1350 and may create a permeabilization buffer that allows analyte derivatives (e.g., connected probes) to diffuse from the biological sample 1302 toward the capture probes of the second substrate (e.g., slide 1306). In some aspects, flow of the permeabilization buffer may deflect transcripts and/or molecules from the biological sample 1302 and may affect diffusive transfer of analytes for spatial analysis. A partially or fully sealed chamber 1350 resulting from the one or more spacers 1310, the first substrate, and the second substrate may reduce or prevent flow from undesirable convective movement of transcripts and/or molecules over the diffusive transfer from the biological sample 1302 to the capture probes.

In some instances, the first substrate and the second substrate are arranged in an angled sandwich assembly as described herein. For example, during the sandwiching of the two substrates (e.g., the slide 1103 and the slide 1104), an angled closure workflow may be used to suppress or eliminate bubble formation.

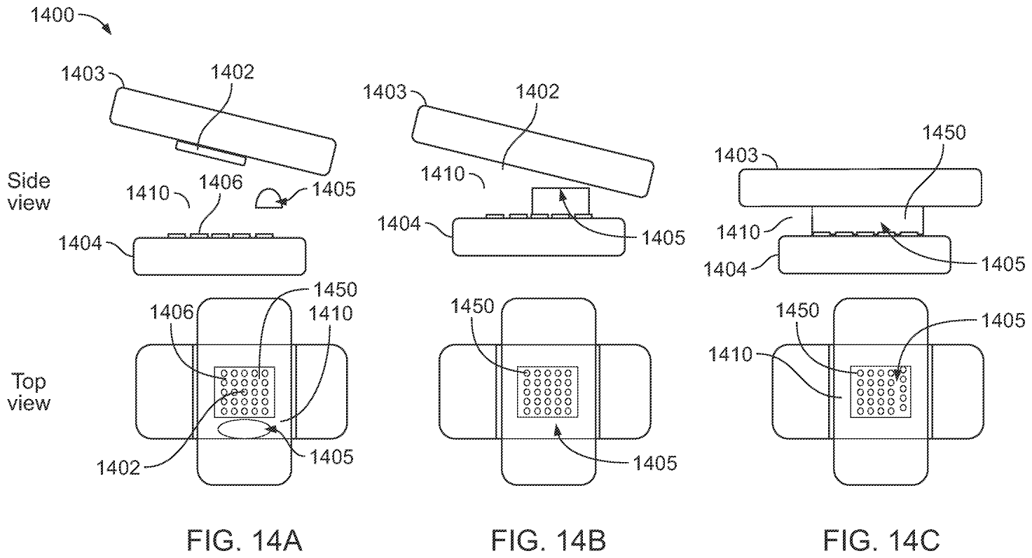

FIGS. 14A-14C depict a side view and a top view of an exemplary angled closure workflow 1400 for sandwiching a first substrate (e.g., slide 1103) having a biological sample 1102 and a second substrate (e.g., slide 1104 having capture probes 1106) in accordance with some example implementations.

FIG. 14A depicts the first substrate (e.g., the slide 1403 including biological sample 1402) angled over (superior to) the second substrate (e.g., slide 1404). As shown, a drop of the reagent medium (e.g., permeabilization solution) 1405 is located on the spacer 1410 toward the right-hand side of the side view in FIG. 14A. While FIG. 14A depicts the reagent medium on the right hand side of side view, it should be understood that such depiction is not meant to be limiting as to the location of the reagent medium on the spacer.

FIG. 14B shows that as the first substrate lowers, and/or as the second substrate rises, the dropped side of the first substrate (e.g., a side of the slide 1403 angled toward the second substrate) may contact the drop of the reagent medium 1405. The dropped side of the first substrate may urge the reagent medium 1405 toward the opposite direction (e.g., towards an opposite side of the spacer 1410, towards an opposite side of the first substrate relative to the dropped side). For example, in the side view of FIG. 14B the reagent medium —1405 may be urged from right to left as the sandwich is formed.

In some embodiments, the first substrate and/or the second substrate are further moved to achieve an approximately parallel arrangement of the first substrate and the second substrate.

FIG. 14C depicts a full closure of the sandwich between the first substrate and the second substrate with the spacer 1410 contacting both the first substrate and the second substrate and maintaining a separation distance and optionally the approximately parallel arrangement between the two substrates. As shown in the top view of FIG. 14C, the spacer 1410 fully encloses and surrounds the biological sample 1402 and the capture probes 1406, and the spacer 1410 forms the sides of chamber 1450 which holds a volume of the reagent medium 1405.

It should be understood that while FIGS. 14A-14C depict the first substrate (e.g., the slide 1403 including biological sample 1402) angled over (superior to) the second substrate (e.g., slide 1404) and the second substrate comprising the spacer 1410, it should be understood that an exemplary angled closure workflow can include the second substrate angled over (superior to) the first substrate and the first substrate comprising the spacer 1410.

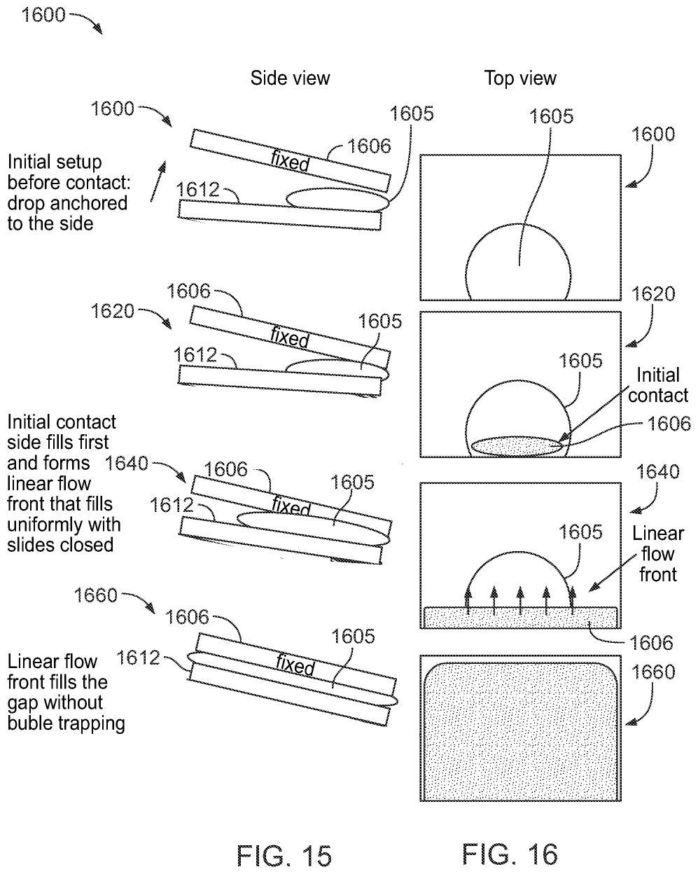

FIG. 15 is a side view of the angled closure workflow 1500 in accordance with some example implementations. FIG. 16 is a top view of the angled closure workflow 1500 in accordance with some example implementations. As shown at step 1600, the drop of reagent medium 1605 is positioned to the side of the substrate 1612.

At step 1620, the dropped side of the angled substrate 1606 contacts the drop of reagent medium 1605 first. The contact of the substrate 1606 with the drop of reagent medium 1605 may form a linear or low curvature flow front that fills uniformly with the slides closed.

At step 1640 the substrate 1606 is further lowered toward the substrate 1612 (or the substrate 16512 is raised up toward the substrate 1606) and the dropped side of the substrate 1606 may contact and may urge the liquid reagent toward the side opposite the dropped side and creating a linear or low curvature flow front that may prevent or reduce bubble trapping between the slides.

At step 1660, the drop of reagent medium 1605 fills the gap (e.g., the gap shown in 1107) between the substrate 1606 and the substrate 1612. The linear flow front of the liquid reagent may form by squeezing the drop 1605 volume along the contact side of the substrate 1612 and/or the substrate 1606. Additionally, capillary flow may also contribute to filling the gap area.

In some aspects, an angled closure workflow disclosed herein (e.g., FIGS. 14A-14C, 15, and -16) may be performed by a sample handling apparatus (e.g., as described in WO 2022/061152, which is hereby incorporated by reference in its entirety.

Further details on angled closure workflows, and devices and systems for implementing an angled closure workflow, are described in WO 2021/252747 and WO 2022061152, which are hereby incorporated by reference in their entirety.

Additional configurations for reducing or eliminating bubble formation, and/or for reducing unwanted fluid flow, are described in WO 2021252747A1, which is hereby incorporated by reference in its entirety.

In some embodiments, the reagent medium comprises a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). Exemplary permeabilization reagents are described in in US. Patent Application Pub. No. 20210189475, which is incorporated by reference in its entirety.

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Exemplary lysis reagents are described in US. Patent Application Pub. No. 20210189475, which is incorporated by reference in its entirety.

In some embodiments, the reagent medium comprises a protease. Exemplary proteases include, e.g., pepsin, trypsin, pepsin, elastase, and proteinase K. Exemplary proteases are described in US. Patent Application Pub. No. 20210189475, which is incorporated by reference in its entirety.

In some embodiments, the reagent medium comprises a detergent. Exemplary detergents include sodium dodecyl sulfate (SDS), sarkosyl, saponin, Triton X-100™, and Tween-20T™. Exemplary detergents are described in US. Patent Application Pub. No. 20210189475, which is incorporated by reference in its entirety.

In some embodiments, the reagent medium comprises a nuclease. In some embodiments, the nuclease comprises am RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNAse, and a sodium salt thereof.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

The sample holder is compatible with a variety of different schemes for contacting the aligned portions of the biological sample and array with the reagent medium to promote analyte capture. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap 307 while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some instances, the aligned portions of the biological sample and the array are in contact with the reagent medium 1105 for about 1 minute. In some instances, the aligned portions of the biological sample and the array are in contact with the reagent medium 1105 for about 5 minutes. In some instances, the aligned portions of the biological sample and the array are in contact with the reagent medium 1105 in the gap 1107 for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium 1105 for about 1-60 minutes. In some instances, the aligned portions of the biological sample and the array are in contact with the reagent medium 1105 for about 30 minutes.

In some embodiments, following initial contact between sample and a permeabilization agent, the permeabilization agent can be removed from contact with sample (e.g., by opening sample holder).

In some instances, the device is configured to control a temperature of the first and second substrates. In some embodiments, the temperature of the first and second members is lowered to a first temperature that is below room temperature (e.g., 25 degrees Celsius) (e.g., degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes that are released from the permeabilized tissue of the sample diffuse to the surface of the second substrate and are captured on the array (e.g., barcoded probes) of the second substrate. In a fourth step, the first substrate and the second substrate are separated (e.g., pulled apart) and temperature control is stopped.

In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of analytes from the sample to the array.

In certain embodiments, different permeabilization agents or different concentrations of permeabilization agents can be infused into array features (e.g., beads) or into a hydrogel layer as described above. By locally varying the nature of the permeabilization reagent(s), the process of analyte capture from the sample can be spatially adjusted.

In some instances, migration of the analyte from the biological sample to the second substrate is passive (e.g., via diffusion). Alternatively, in certain embodiments, migration of the analyte from the biological sample is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher analyte capture efficiency and better spatial fidelity of captured analytes (e.g., on a feature array) than random diffusion onto matched substrates without the application of an electric field (e.g., via manual alignment of the two substrates). Exemplary methods of electrophoretic migration are described in WO 2020/176788, including at FIGS. 13-15, 24A-24B, and 25A-25C of WO 2020/176788, which is hereby incorporated by reference in its entirety.

Loss of spatial resolution can occur when analytes migrate from the sample to the feature array and a component of diffusive migration occurs in the transverse (e.g., lateral) direction, approximately parallel to the surface of the first substrate on which the sample is mounted. To address this loss of resolution, in some embodiments, a permeabilization agent deposited on or infused into a material with anisotropic diffusion can be applied to the sample or to the feature array. The first and second substrates are aligned by the sample holder and brought into contact. A permeabilization layer that includes a permeabilization solution infused into an anisotropic material is positioned on the second substrate.

In some embodiments, the feature array can be constructed atop a hydrogel layer infused with a permeabilization agent. The hydrogel layer can be mounted on the second substrate, or alternatively, the hydrogel layer itself may function as the second substrate. When the first and second substrates are aligned, the permeabilization agent diffuses out of the hydrogel layer and through or around the feature array to reach the sample. Analytes from the sample migrate to the feature array. Direct contact between the feature array and the sample helps to reduce lateral diffusion of the analytes, mitigating spatial resolution loss that would occur if the diffusive path of the analytes was longer.

Spatial analysis workflows can include a sandwiching process described herein, e.g., a process as described in FIG. 11. In some embodiments, the workflow includes provision of the first substrate comprising the biological sample. In some embodiments, the workflow includes mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

The biological sample can be stained using known staining techniques, including, without limitation, Can-Grunwald, Giemsa, hematoxylin and eosin (H&E), hematoxylin, Jenner's, Leishman. Masson's trichrome, Papanicolaou, Romanowsky, silver, Sudan, Wright's, and/or Periodic Acid Schiff (PAS) staining techniques. PAS staining is typically performed after formalin or acetone fixation. In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes biological staining using hematoxylin. In some embodiments, staining includes identifying analytes using fluorescently-conjugated antibodies, e.g., by immunofluorescence. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample. In some instances, a biological sample on the first substrate is stained.

In some instances, methods for immunofluorescence include a blocking step. The blocking step can include the use of blocking probes to decrease unspecific binding of the antibodies. The blocking step can optionally further include contacting the biological sample with a detergent. In some instances, the detergent can include Triton X-100™. The method can further include an antibody incubation step. In some embodiments, the antibody incubation step effects selective binding of the antibody to antigens of interest in the biological sample. In some embodiments, the antibody is conjugated to an oligonucleotide (e.g., an oligonucleotide-antibody conjugate as described herein). In some embodiments, the antibody is not conjugated to an oligonucleotide. In some embodiments, the method further comprises an antibody staining step. The antibody staining step can include a direct method of immunostaining in which a labelled antibody binds directly to the analyte being stained for. Alternatively, the antibody staining step can include an indirect method of immunostaining in which a first antibody binds to the analyte being stained for, and a second, labelled antibody binds to the first antibody. In some embodiments, the antibody staining step is performed prior to sandwich assembly. In some embodiments wherein an oligonucleotide-antibody conjugate is used in the antibody incubation step, the method does not comprise an antibody staining step.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, image are captured using high resolution techniques (e.g., having 300 dots per square inch (dpi) or greater). For example, images can be captured using brightfield imaging (e.g., in the setting of hematoxylin or H&E stain), or using fluorescence microscopy to detect adhered labels. In some instances, high resolution images are captured temporally using e.g., confocal microscopy. In some instances, a low resolution image is captured. A low resolution image (e.g., images that are about 72 dpi and normally have an RGB color setting) can be captured at any point of the workflow, including but not limited to staining, destaining, permeabilization, sandwich assembly, and migration of the analytes. In some instances, a low resolution image is taken during permeabilization of the biological sample.

In some embodiments, the location of the one or more analytes in a biological sample are determined by immunofluorescence. In some embodiments, one or more detectable labels (e.g., fluorophore-labeled antibodies) bind to the one or more analytes that are captured (hybridized to) by a probe on the first slide and the location of the one or more analytes is determined by detecting the labels under suitable conditions. In some embodiments, one or more fluorophore-labeled antibodies are used to conjugate to a moiety that associates with a probe on the first slide or the analyte that is hybridized to the probe on the first slide. In some instances, the location(s) of the one or more analytes is determined by imaging the fluorophore-labeled antibodies when the fluorophores are excited by a light of a suitable wavelength. In some embodiments, the location of the one or more analytes in the biological sample is determined by correlating the immunofluorescence data to an image of the biological sample. In some instances, the tissue is imaged throughout the permeabilization step.

In some instances, the biological samples can be destained. In some instances, destaining occurs prior to permeabilization of the biological sample. By way of example only, H&E staining can be destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

In some instances, after the sandwiching process the first substrate and the second substrate are separated (e.g., such that they are no longer aligned in a sandwich configuration, also referred to herein as opening the sandwich). In some embodiments, subsequent analysis (e.g., cDNA synthesis, library preparation, and sequences) can be performed on the captured analytes after the first substrate and the second substrate are separated.

### (e) Systems and Kits

Also disclosed herein are systems and kits used for any one of the methods disclosed herein. In some instances, the system of kit is used for analyzing an analyte in a biological sample. In some instances, the system or kit includes a support device configured to retain a first substrate and a second substrate, wherein the biological sample is placed on the first substrate, and wherein the second substrate comprises a plurality of capture probes, wherein a capture probe of the plurality of capture probes comprises: (i) a spatial barcode and (ii) a capture domain. In some instances, the system or kit includes a first probe oligonucleotide and a second probe oligonucleotide, wherein the first probe oligonucleotide and the second probe oligonucleotide each comprise a sequence that is substantially complementary to adjacent sequences of the analyte, wherein the second probe oligonucleotide comprises a capture probe binding domain, and wherein the first probe oligonucleotide and the second probe oligonucleotide are capable of being ligated together to form a connected probe. In some instances, the system or kit includes a plurality of analyte capture agents, wherein an analyte capture agent of the plurality of analyte capture agents comprises an analyte binding moiety and a capture agent barcode domain, wherein the analyte binding moiety specifically binds to the analyte, and wherein the capture agent barcode domain comprises an analyte binding moiety barcode and a capture handle sequence. In some instances, the system or kit further includes a reagent medium comprising a permeabilization agent and optionally an agent for releasing the connected probe. In some instances, the system or kit includes instructions for performing any one of the methods described herein.

Also provided herein are systems or kits for analyzing an analyte in a fixed biological sample, the system or the kit comprising (a) a support device configured to retain a first substrate and a second substrate, wherein the fixed biological sample is placed on the first substrate, and wherein the second substrate comprises a plurality of capture probes, wherein a capture probe of the plurality of capture probes comprises: (i) a spatial barcode and (ii) a capture domain; (b) (b1) a first probe and a second probe, wherein the first probe and the second probe each comprise a sequence that is substantially complementary to a first sequence and a second sequence of the analyte, wherein the second probe comprises a capture probe binding domain, and wherein the first probe and the second probe are capable of being ligated together to form a connected probe; and/or (b2) a plurality of analyte capture agents, wherein an analyte capture agent of the plurality of analyte capture agents comprises an analyte binding moiety and a capture agent barcode domain, wherein the analyte binding moiety specifically binds to the analyte, and wherein the capture agent barcode domain comprises an analyte binding moiety barcode and a capture handle sequence; (c) reagents for determining the presence of RNA of sufficient integrity in the fixed biological sample; (d) a reagent medium comprising a permeabilization agent and optionally an agent for releasing the connected probe; and (e) instructions for performing any one of the methods described herein.

In some instances, the permeabilization agent is pepsin or proteinase K. In some instances, the agent for releasing the connected probe is an RNAse, optionally wherein the RNAse is RNAse H.

In some instances, the system or kit further includes an alignment mechanism on the support device to align the first substrate and the second substrate. In some instances, the alignment mechanism comprises a linear actuator and the first substrate comprises a first member and the second substrate comprises a second member. The linear actuator can be configured to move the second member along an axis orthogonal to the plane or the first member and/or the second member. The linear actuator can be configured to move the first member along an axis orthogonal to the plane of the first member and/or the second member. The linear actuator can be configured to move the first member, the second member, or both the first member and the second member at a velocity of at least 0.1 mm/sec. Finally, in some instances, the linear actuator can be configured to move the first member, the second member, or both the first member and the second member with an amount of force of at least 0.1 lbs.

### Example 1—RNA Integrity Assays on Fixed Samples

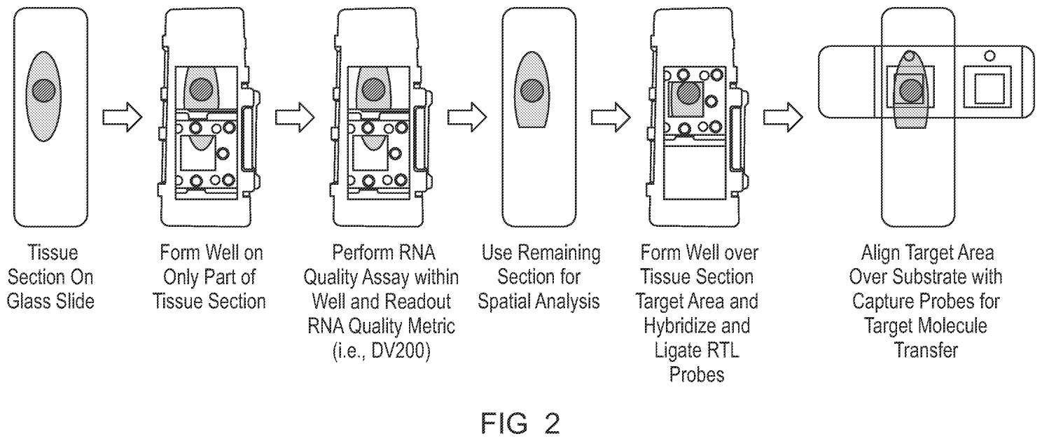

In a non-limiting example, RNA integrity assays were performed on FFPE samples from a human brain tumor tissue sample. FIG. 2 shows an exemplary schematic of the workflow of performing the RNA integrity assays on a portion of the FFPE sample of a substrate. Briefly, after sectioning a tissue sample (e.g., about 5 μm thick) and placing the sample on a glass slide, part of the tissue sample is sectioned from the rest of the sample for purposes of determining RNA quality. RNA integrity of a portion of a fixed tissue section was assessed as follows. First, the slide was baked at 60° C. for 30 minutes. The slide was then incubated in Xylene for 10 mins, followed by incubation in absolute ethanol, 96% ethanol, and 70% ethanol to rinse the slide. The slide was placed in a cassette and a gasket was moved to a region of interest in the tissue section on the slide to be evaluated for RNA integrity, thereby forming a well containing the region of interest. The isolation of RNA from the region of interest in the tissue section was performed with the RNeasy FFPE kit from Qiagen (Catalog ID No.: 73504) generally following the manufacturer's instructions. Buffer PKD and proteinase K were added to the well containing the region of interest and the cassette was incubated at 56° C. for 15 minutes on a thermocycler. The cassette was then incubated at 80° C. for 15 minutes on a thermocycler and the contents were transferred to a microcentrifuge tube. The tube was incubated in ice and centrifuged at 20,000 g for 15 minutes, wherein the supernatant was transferred to a fresh centrifuge tube. DNase Booster buffer (16 μL) and DNase I solution (10 μL) were added and incubated at room temperature for 15 minutes. Buffer RBC (320 μL) and absolute ethanol (720 μL) were added into the tube and mixed well. The sample was transferred to RNeasy MinElute spin column and spun at 8000 g for 15 seconds. Buffer RPE was added after the spinning and the RNA was eluted with RNAse free water and DV200 scores were measured. In some embodiments, DV200 scores were determined using 1 μL of the RNA sample on an Agilent Tapestation system.

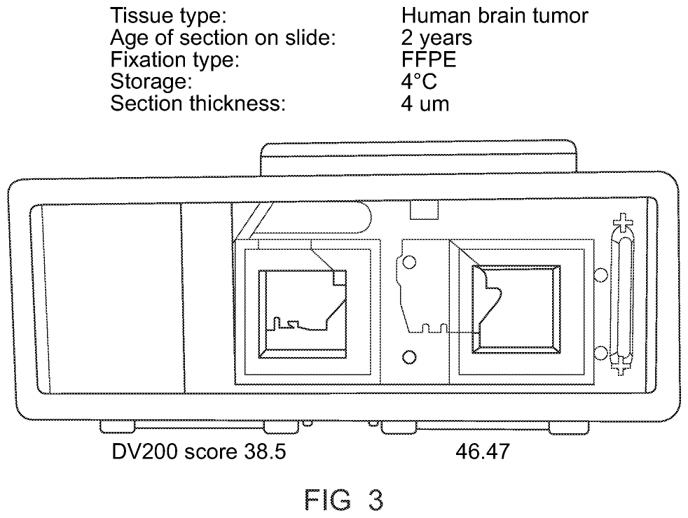

FIG. 3 shows DV200 scores in FFPE samples from human brain tumor tissue, wherein the FFPE sample was stored on the slide for 2 years at 4° C. Results show that the sample on the right (in which the well was formed over an outer edge of the tissue) had a higher DV200 score of 46.47, while the sample on the left (in which the well was formed over a central portion of the tissue) had a DV200 score of 38.5, suggesting that there is small variation in different parts of tissue samples.

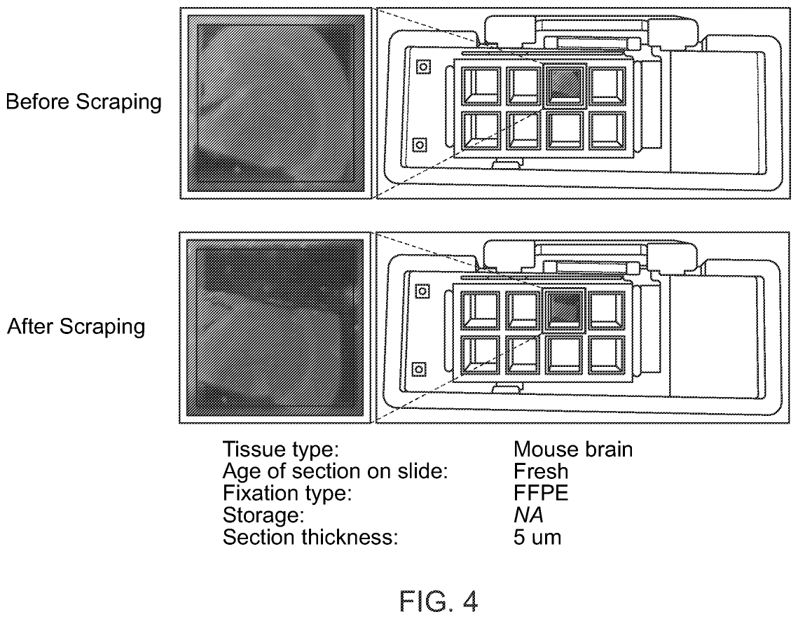

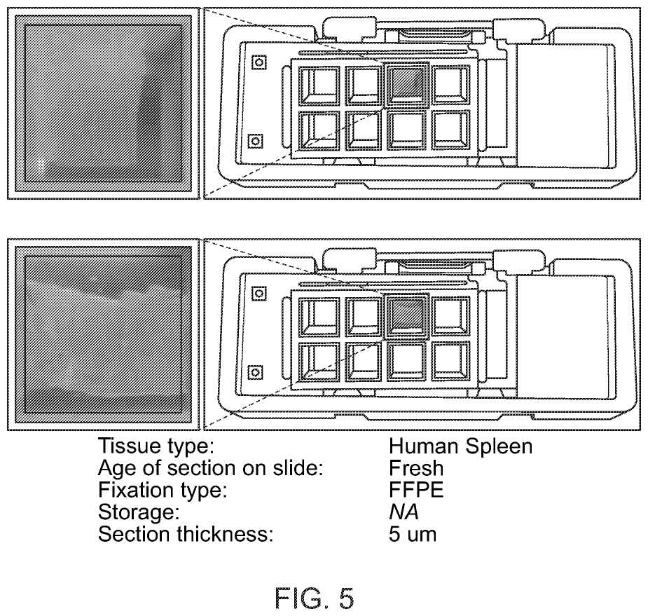

In another non-limiting example, RNA integrity assays were performed on FFPE samples from mouse brain (FIG. 4) and human spleen (FIG. 5) tissue samples. The assays were performed on portion of the tissue samples on slide as illustrated in FIG. 2 (upper panel) and portion of the tissue samples that were first scraped into a tube (lower panel), wherein the results show that RNA integrity assays yielded comparable DV200 scores in each sample (Table 1).

TABLE 1
DV200 scores.

| Sample | DV200 Score |
|---|---|
| Brain (in cassette) | 55 |
| Brain (scraped into tube) | 56 |
| Spleen (in cassette) | 50 |
| Spleen (scraped into tube) | 58 |

After determining that the quality of the RNA in a sample is sufficient for downstream purposes, gene expression assays can be performed. For instance, in a non-limiting example, the remaining portion of the FFPE sections on standard slides (for sandwich conditions) or gene expression (GEx) slides containing capture probes (for non-sandwich control conditions) are subject to spatial transcriptomic analysis. For example, in some embodiments, the FFPE tissue sections are deparaffinized, H&E stained, and imaged. Next, the tissue sections are hematoxylin-destained with three HCl solution washes. The sections are then decrosslinked by incubating at 70° C. for 1 hour in TE pH 9.0. The TE is removed and the tissues are incubated in 1×PBS-Tween for 15 minutes.

Individual RTL probes (e.g., a first probe and a second probe) of probe pairs are hybridized to sequences of an analyte (e.g., an RNA molecule) in the tissue. The RTL probes are then ligated together, thereby creating a connected probe (e.g., a ligation product). The connected probe (e.g., a ligation product) includes a capture probe binding domain. In some embodiments, the probes are designed to hybridize to each transcript in the transcriptome or to majority of the transcriptome.

After ligation of the RTL probes, the connected probes are released from the tissue and the connected probes are captured by capture probes (e.g., as shown in FIG. 1). Following permeabilization and capture, the capture probes are extended, sequencing libraries are prepared and sequenced, and the results are analyzed computationally.

### Example 2—Capturing Ligation Products from Room Temperature Stored Lymph Nodes

In a non-limiting example, FFPE sections of human lymph nodes with reactive follicular hyperplasia were placed on standard slides, deparaffinized, H&E stained, coverslipped, and stored at room temperature for about 6 months. For comparison, additional samples were sectioned and mounted onto standard slides and were processed within 1 day of sectioning.

Slides were baked at 60° C. and dipped in xylene to remove the coverslips, followed by incubating the tissues in xylene and decreasing concentrations of ethanol washes, finally washing the deparaffinized tissue sections prior to staining. H&E staining was performed on the tissue sections as is known in the art, followed by imaging of the stained tissue sections.

Following imaging, the tissue sections were destained on a thermocycler by adding a 0.1 N HCl solution to the tissue sections, incubating at 42° C. for 15 minutes, followed by decrosslinking with a citrate in a PBS-Tween buffer at pH 6.0 at 95° C. for 60 minutes.

After decrosslinking, the tissue sections were stained and imaged via immunofluorescence. The tissue sections were queried for the presence of Ki67 protein using anti-Ki67 primary rabbit monoclonal (SP6) antibody conjugated with AlexaFluor 488 fluorescent moiety (AbCam) using established protocols. Fluorescence imaging was performed to identify the location of Ki67 in the tissue sections and correlate Ki67 protein location with mRNA gene expression.

After immunofluorescence and imaging, hybridization probes were added to the tissue sections. For each nucleic acid target, two probes that hybridize to adjacent sequences were used to target a nucleic acid. The PBS-T buffered solution was removed and a FFPE hybridization solution comprising 2.4 nM each of the two probes was added to the tissue sections and incubated overnight at 50° C.

After the overnight incubation, the tissue sections were washed several times with a post-hybridization wash solution that includes SSC at 50° C. for about 5 minutes for each wash. The wash solution was removed and the two probes hybridized to the target nucleic acid sequences were ligated together by addition of a ligation mix to the tissue sections and incubation at 37° C. for 1 hr. The tissue sections were washed several times by adding ligation wash buffer and incubating the tissue sections at 57° C. for 5 minutes. A final wash of the tissue sections in SSC solution at 57° C. for 5 minutes prior to room temperature incubation completed the ligation reaction.

Post probe ligation, the ligation products from the standard slide were transferred via the sandwich method as described herein to a second slide containing a spatial array including capture probes including spatial barcodes and capture domains. The sandwich transfer was performed as described previously and as described in WO 2022/140028.

Following transfer of the ligation products from the standard slide to the spatial array, captured ligation products were used as templates for generating extension products from the capture probes (e.g., extended capture probes) and extending the ligation products using the capture probe as a template to include the sequences of the capture probe, including the spatial barcode. Sequencing libraries were prepared and sequenced, and the results were analyzed computationally.

Two different sections of a lymph node sample block were examined. Ligated paired probes that were transferred from the original standard slide to the array slide including capture probes using the sandwiching process methods showed, on average, 2,718 genes/spot at a sequencing depth of 10k

Table 2 shows a comparison of sequencing metrics for the aged lymph node sections stored on slides for 6 months (i.e., aged sample on XL slides) as compared to sections mounted on glass slides and processed within a day (i.e., regular sample).

TABLE 2
Lymph Node Spatial Results.

| | Aged Sample on | Regular |
|---|---|---|
| Readout | XL Slides | Sample |
| Valid Barcodes | 99.0% | 98.9% |
| Valid UMIs | 100.0% | 100.0% |
| Reads in Spot | 95.0% | 95.8% |
| under Tissue | | |
| Reads Usable | 90.9% | 91.3% |
| Reads Mapped | 71.4% | 70.8% |
| to Transcriptome | | |
| Reads Unmapped | 1.2% | 1.0% |

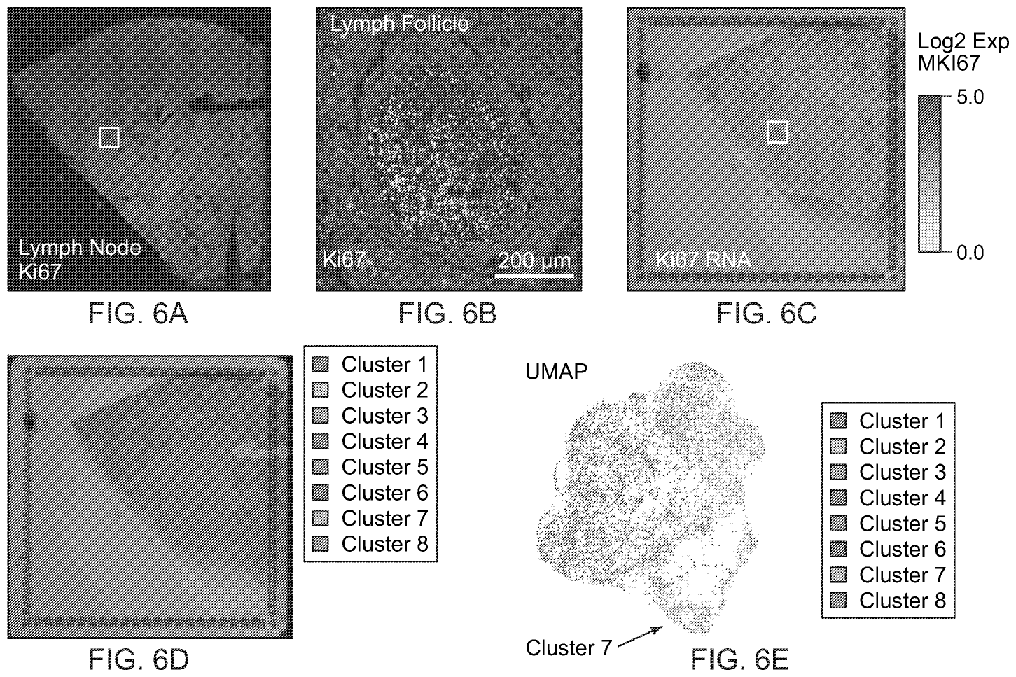

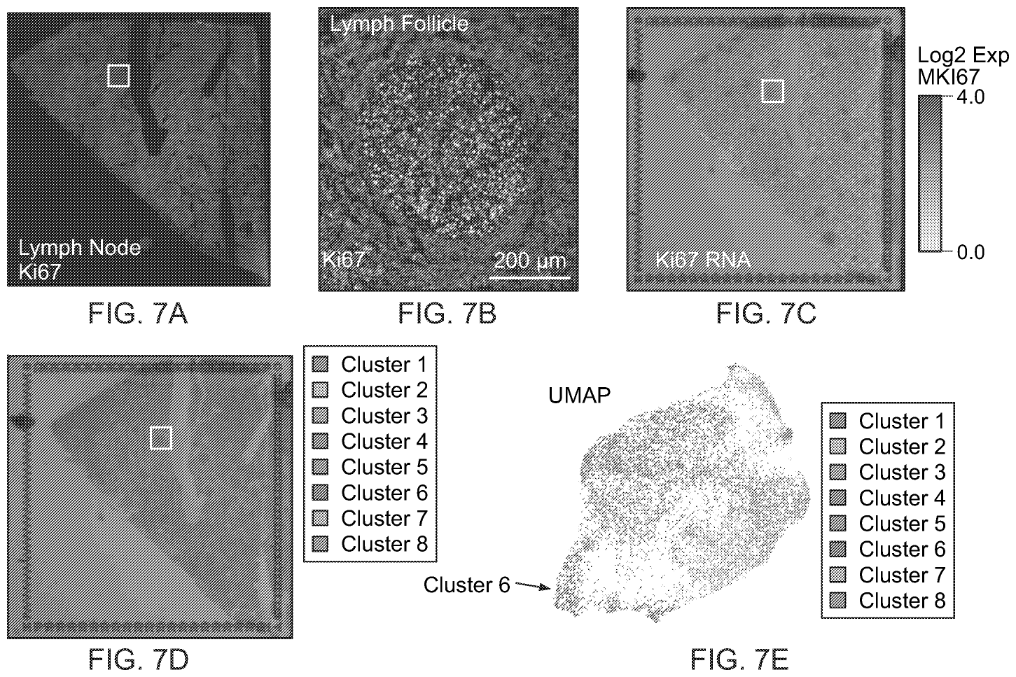

FIGS. 6A, 611, 7A, and 7B, show exemplary Ki67 protein expression detected in the lymph node and in particular in the lymph follicle, demonstrating areas of proliferation in these samples. Ki67 protein expression in both samples overlapped with Ki67 RNA detection determined using templated ligation (FIGS. 6C and 7C).

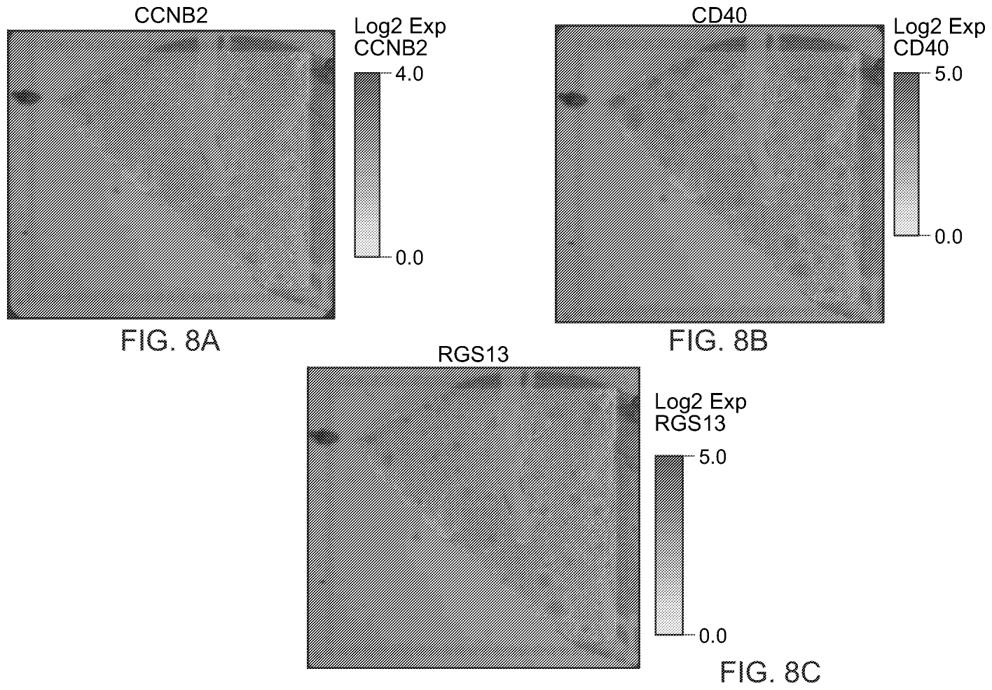

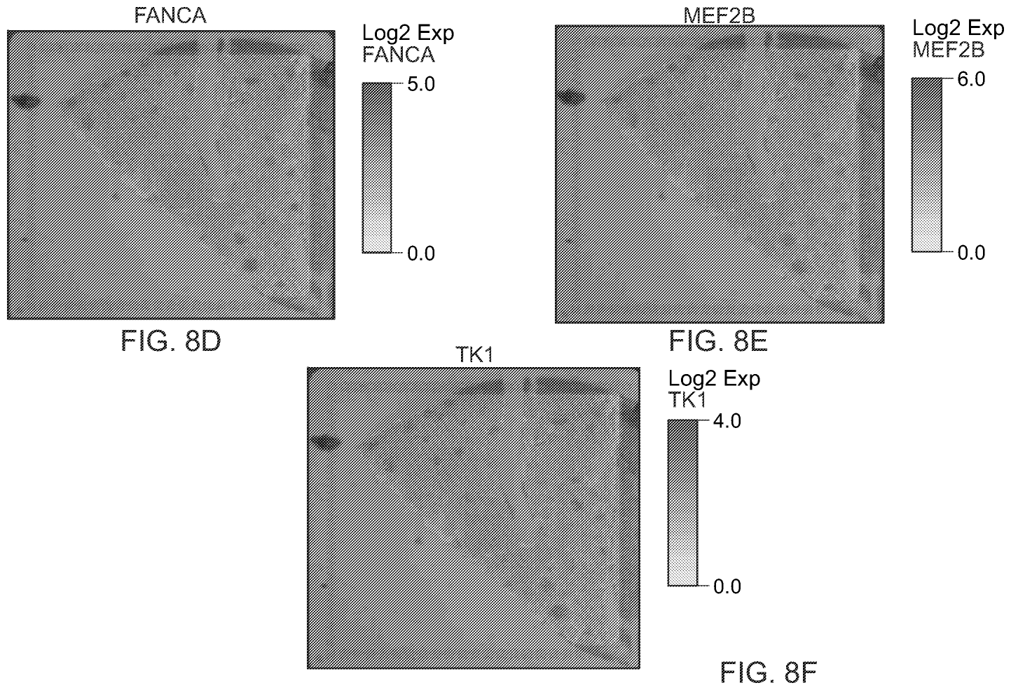

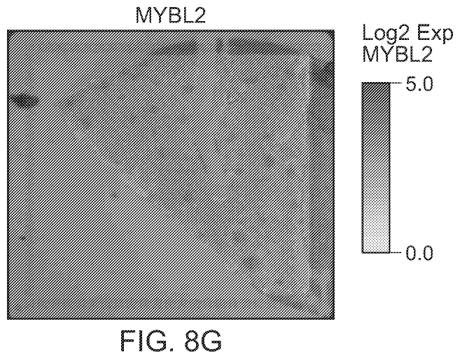

Analyte expression derived from the results of transcriptome templated ligation revealed numerous clusters of gene expression, as shown in FIGS. 6D, 6E, 7D, and 7E. Certain clusters showed overlapping expression with Ki67. For instance, cluster 6 (FIG. 7E) and cluster 7 (FIG. 6E) correlated with cells expressing Ki67 RNA and protein. Additional gene expression markers were examined in the lymph node samples. For instance, Cyclin B2 expression was detected, suggesting that the lymph follicles positive for Cyclin B2 are actively dividing (FIG. 8A). Further, B cells in germinal centers prominently express RGS13, which is regulated by and co-expressed with CD40 (FIGS. 8C and 8B, respectively).

Proto-oncogene FANCA (Fanconi Anaemia Complementation group A) is highly expressed in lymphoid neoplasms, a tumor that arises from B cells in the germinal centers of lymphoid organs. FANCA was readily detected, as shown in FIG. 8D. MEF2B is a germinal center regulator and a driver oncogene in lymphomagenesis, and was detected in conjunction with the other biomarkers herein identified (FIG. 8E). Finally, additional tumor or metastatic markers such as thymidine kinase 1 (TK1) (FIG. 8F) and MYBL2 were detected (FIG. 8G) in conjunction with the other biomarkers mentioned.

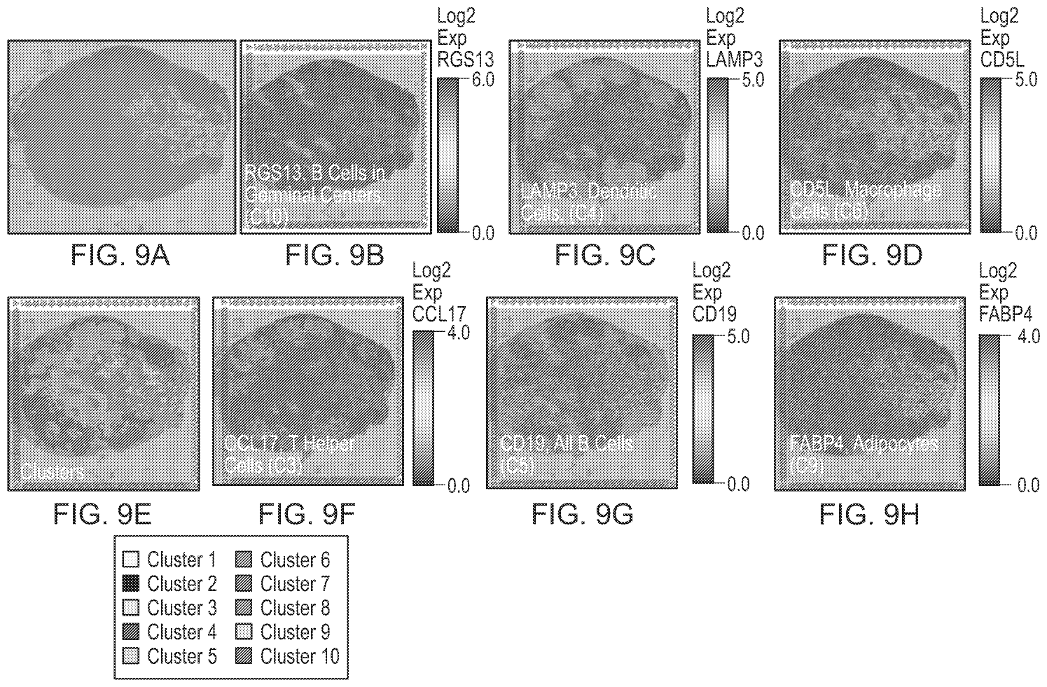

FFPE sections of another human lymph node with reactive follicular hyperplasia that were stored on standard slides at room temperature for about five months were also examined. FIG. 9A shows a representative image of the sample with H&E stain. Five months later after storage at room temperature, the coverslip was removed, and the above methods of analyte detection described in this example were used to examine RNA expression in the sample.

Analyte expression derived from the results of transcriptome templated ligation revealed numerous clusters (n=10) of gene expression, as shown in FIG. 9E. In addition, markers for various cells were readily identified. For instance, RGS13, a marker of B cells in germinal centers, was detected in cluster 10 (FIG. 9B). LAMP3, a marker of dendritic cells, was identified in cluster 4 (FIG. 9C). CD5L, a marker of macrophage cells, was expressed in cluster 6 (FIG. 9D). CCL17, a marker of T Helper cells, was expressed in cluster 3 (FIG. 9F). Finally, CD19 was expressed in all B cells, which was associated with cluster 5 (FIG. 9G), and FABP4 was expressed in adipocytes and associated with cluster 9 (FIG. 9H).

These results demonstrate that the sandwiching methods disclosed herein can advantageously process pre-sectioned FFPE tissue samples that have been stored on slides for spatial analysis, even those samples that have been stored on slides for long periods of time (e.g., 6 months or more) and under suboptimal storage conditions (e.g., storage at room temperature). In this case the tissue samples were human lymph node samples that were diagnosed as reactive follicular hyperplasia and had been archived for about six months at room temperature. As demonstrated herein, FFPE archived samples that have been stored in less than optimal conditions, can still yield usable data for research and discovery efforts.

### Example 3—Capturing Ligation Products from Archived and Room Temperature Stored Lymph Nodes

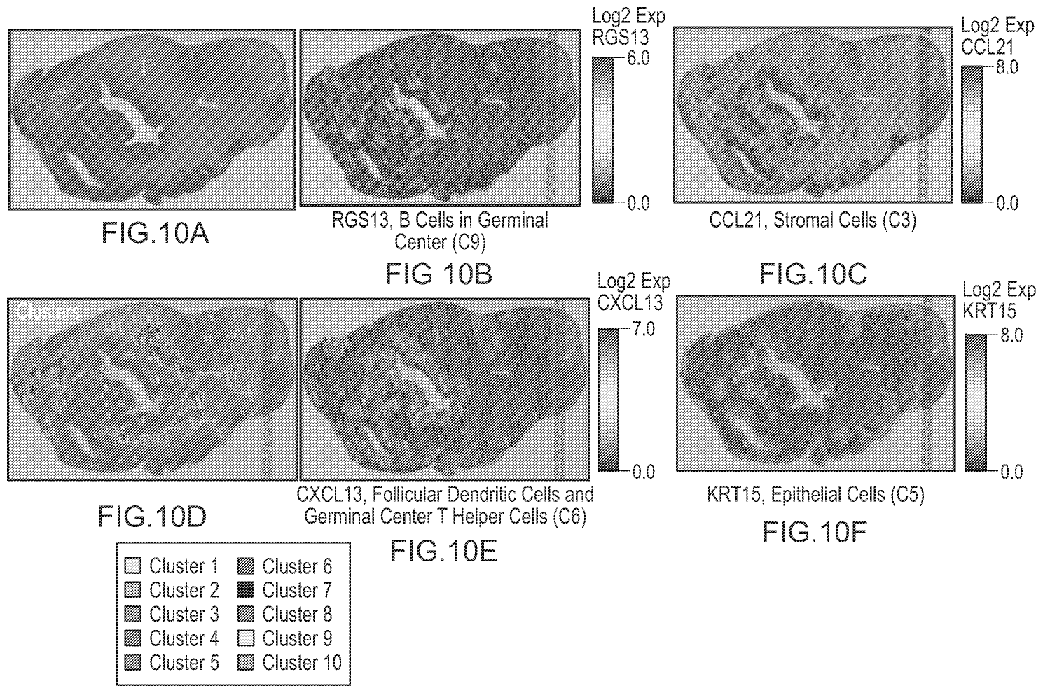

Using the methods described in Example 2, human tonsil sections were examined. The human tonsil section was from a human subject having tonsillitis. The sample was an FFPE section stored on a standard slide at room temperature for about two months. FIG. 10A shows a representative image of the sample H&E stained. Two months later, after storage at room temperature, the coverslip was removed, and the methods of analyte detection described in Example 2 were used to examine RNA expression in the sample.

Analyte expression derived from the results of transcriptome templated ligation revealed numerous clusters (n=10) of gene expression, as shown in FIG. 10D. In addition, markers for various cells were readily identified. For instance, RGS13, a marker of B cells in germinal centers, was detected in cluster 9 (FIG. 10B). CCL21, a marker of stromal cells, was identified in cluster 3 (FIG. 10C). CXCL13, a marker of follicular dendritic cells and germinal center T Helper cells, was expressed in cluster 6 (FIG. 10E). Finally, KRT15 was expressed in epithelial cells, which was associated with cluster 5 (FIG. 10F).

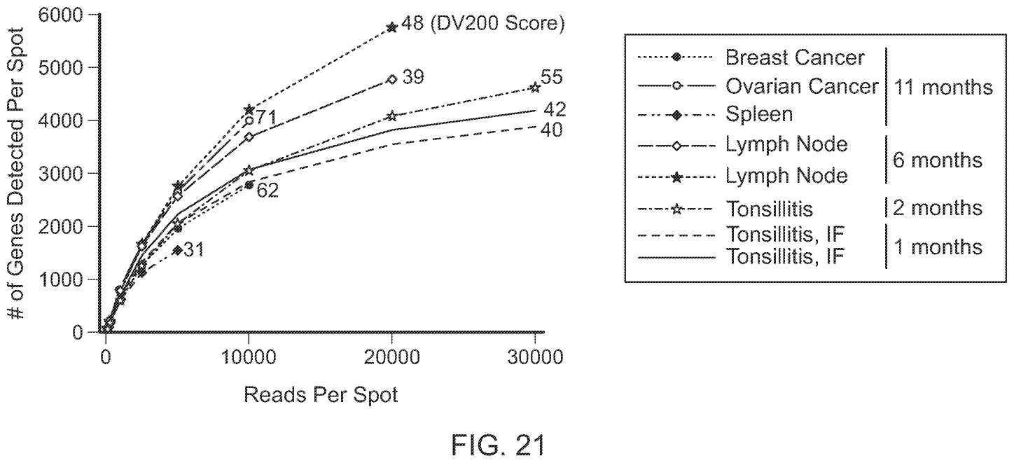

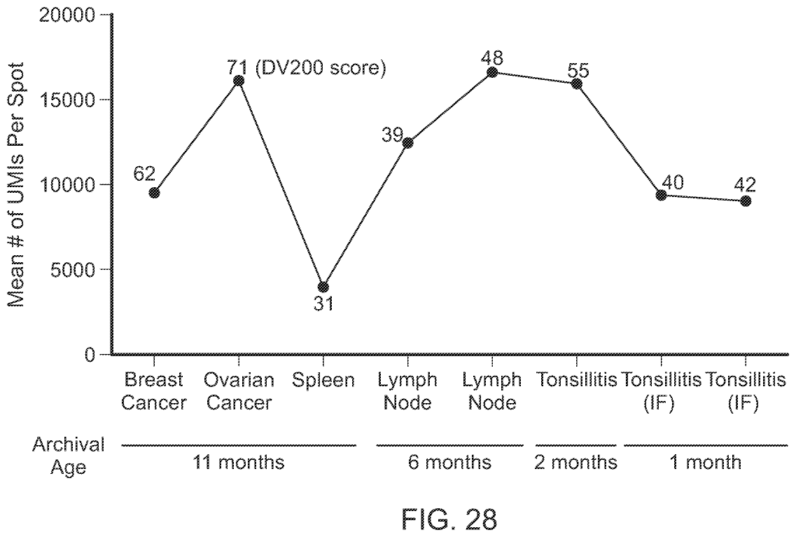

Also, using the methods described in Example 2, sections from a breast cancer sample, an ovarian cancer sample, a spleen sample, and a lymph node sample were examined. The breast cancer sample, ovarian cancer sample, and spleen sample were FFPE sections stored on standard slides at room temperature for about eleven months, and the RNA integrity assays yielded a DV200 score of 62, 71, and 31, respectively. The lymph node samples were FFPE sections stored on a standard slide at room temperature for about 6 months, and the RNA integrity assay yielded DV200 scores of 39 and 48. As shown in FIG. 21, the quality of RNA in each sample at various storage time points still have a threshold DV200 score of above 30 which resulted in the ability to obtain sufficient sequence data at various sequencing depths, using the workflows described herein. Further, RNA quality and sequencing results were not hindered in two samples that were immunofluorescently stained. Furthermore, FIG. 28 depicts mean #UMIs per spot obtained from the various samples, as well as their DV200 score. As shown in FIG. 28, sufficient sequence data can be obtained from archived FFPE sections using the workflows described herein.

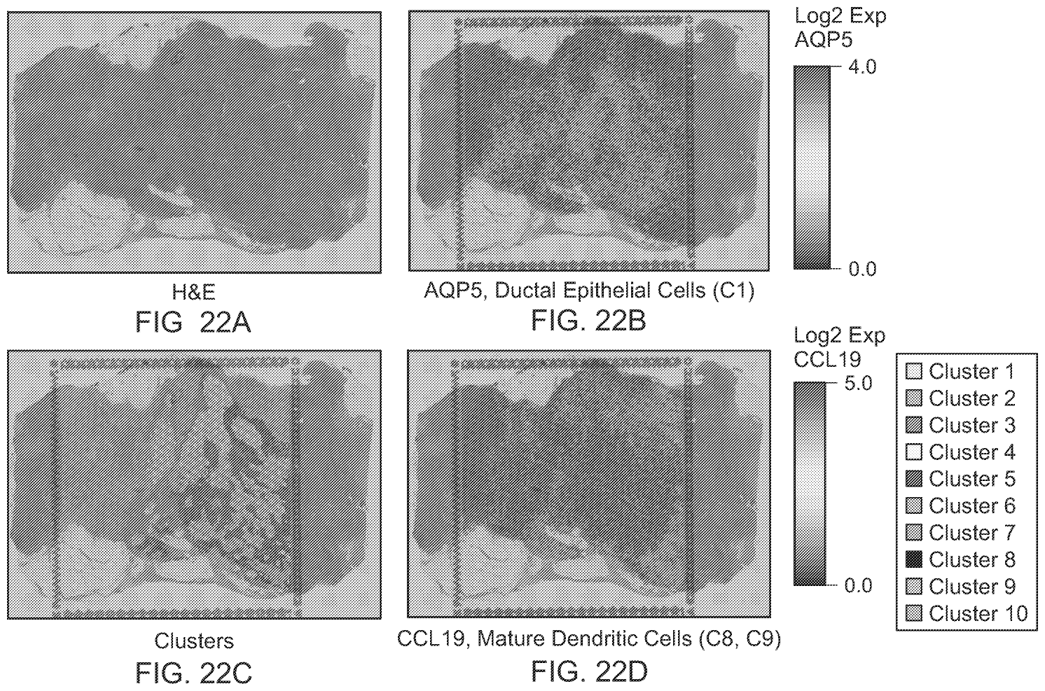

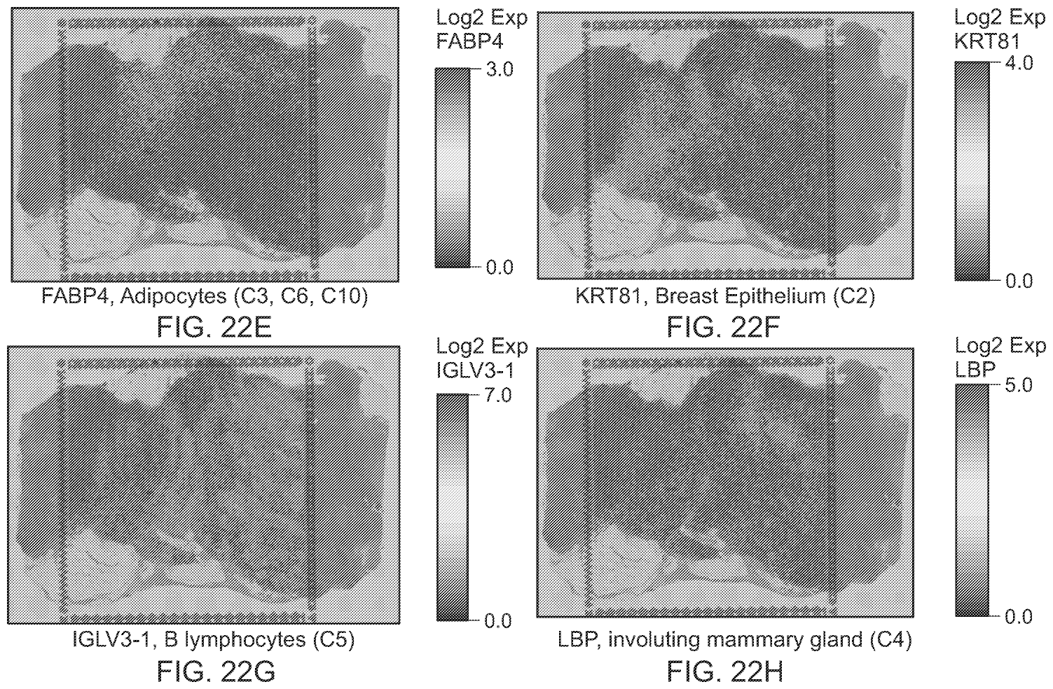

Representative images of the breast cancer sample are shown in FIGS. 22A-22H. For instance, a representative H&E stain is shown in FIG. 22A. Using the methods of spatial analyte detection disclosed herein, one can detect groups of analytes and even individual analytes in a tissue section. As shown in FIG. 22C, UMAP visualization of expression in the breast cancer sample reveals 10 different clusters of cell types. The methods disclosed herein also allow for individual analyte expression to reveal different types of cells expressed in a breast cancer tissue sample. For instance, as shown in FIGS. 22B and 22D-22H, AQP5 (FIG. 22B) was expressed in ductal epithelial cells (differentially expressed in Cluster 1 of FIG. 22C); CCL19 (FIG. 22D) was expressed in mature dendritic cells (differentially expressed in Clusters 8 and 9 of FIG. 22C); FABP4 (FIG. 22E) was expressed in adipocytes (differentially expressed in Clusters 3, 6, and 10 of FIG. 22C); KRT81 (FIG. 22F) was expressed in breast epithelium (differentially expressed in Cluster 2 of FIG. 22C); IGLV3-1 (FIG. 22G) was expressed in B lymphocytes (differentially expressed in Cluster 5 of FIG. 22C); and LBP (FIG. 22H) was expressed in the involuting mammary gland (differentially expressed in Cluster 4 of FIG. 22C). Taken together, these data show that RNA from FFPE breast cancer samples stored for months at room temperature can be evaluated using spatial methods described herein.

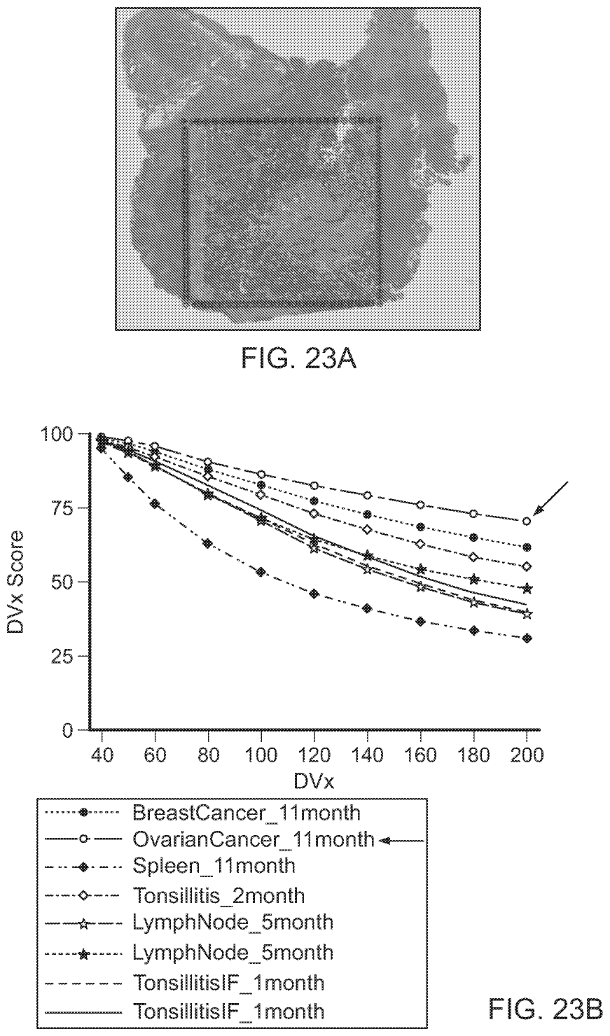

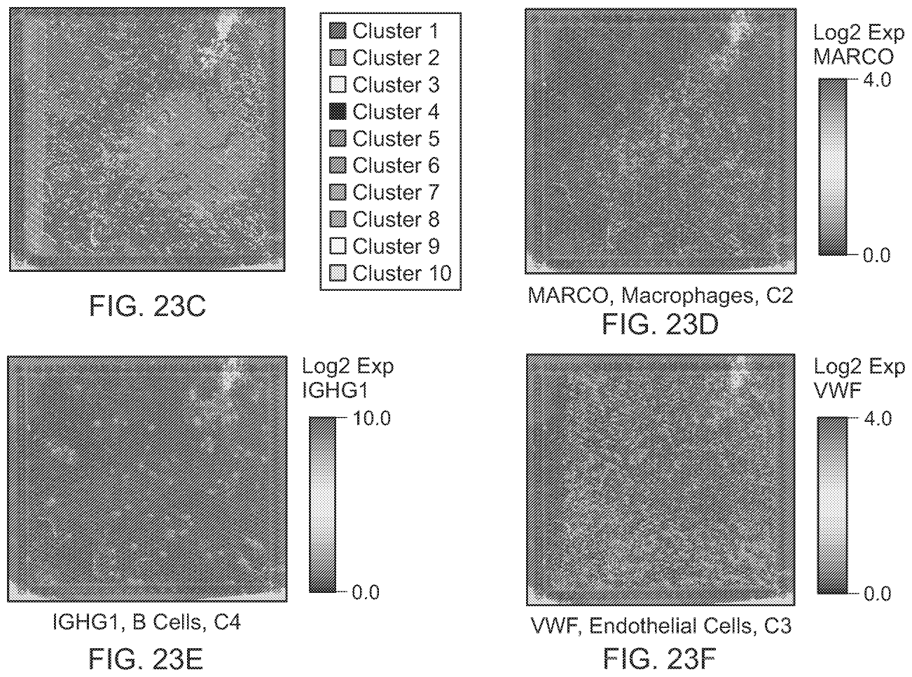

Representative images of the ovarian cancer sample are shown in FIGS. 23A and 23C-23F. For instance, a representative H&E stain is shown in FIG. 23A. Using the methods of spatial analyte detection disclosed herein, one can detect groups of analytes and even individual analytes in a tissue section. As shown in FIG. 23C, UMAP visualization of expression in the ovarian cancer sample reveals 10 different clusters of cell types. The methods disclosed herein also allow for individual analyte expression to reveal different types of cells expressed in an ovarian cancer tissue sample. For instance, as shown in FIGS. 23D-23F, MARCO (FIG. 23D) was expressed in macrophages (differentially expressed in Cluster 2 of FIG. 23C); IGHG1 (FIG. 23E) was expressed in B cells (differentially expressed in Cluster 4 of FIG. 23C); and VWF (FIG. 23F) was expressed in endothelial cells (differentially expressed in Cluster 3 of FIG. 23C). Spatial fragment distribution value (DV) at various thresholds (x-axis) compared to spatial fragment DV score (y-axis) in archived sections stored at RT are also shown in FIG. 23B. Taken together, these data show that RNA from FFPE ovarian cancer samples stored for months at room temperature can be evaluated using spatial methods described herein.

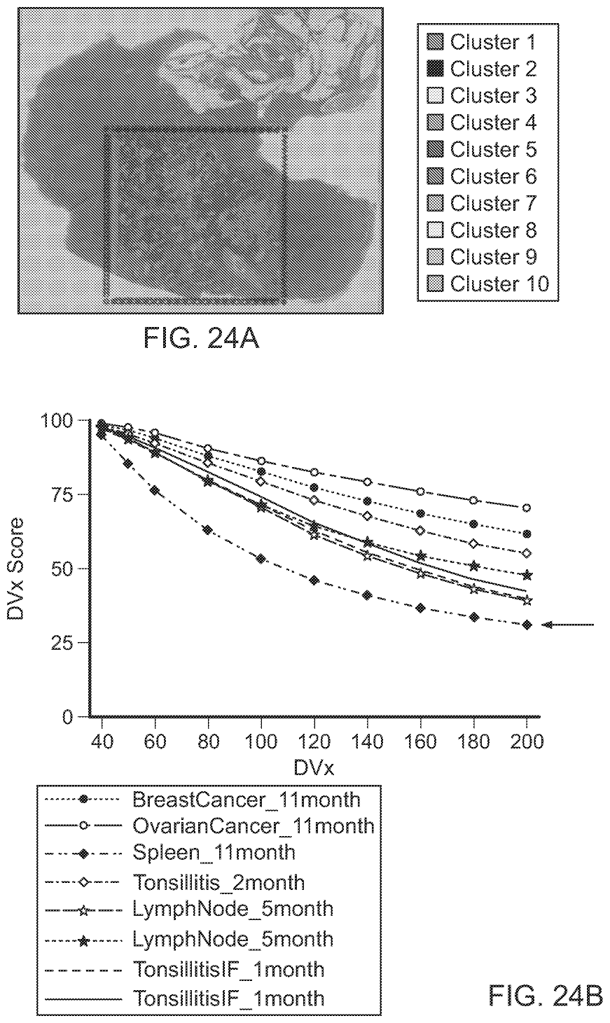

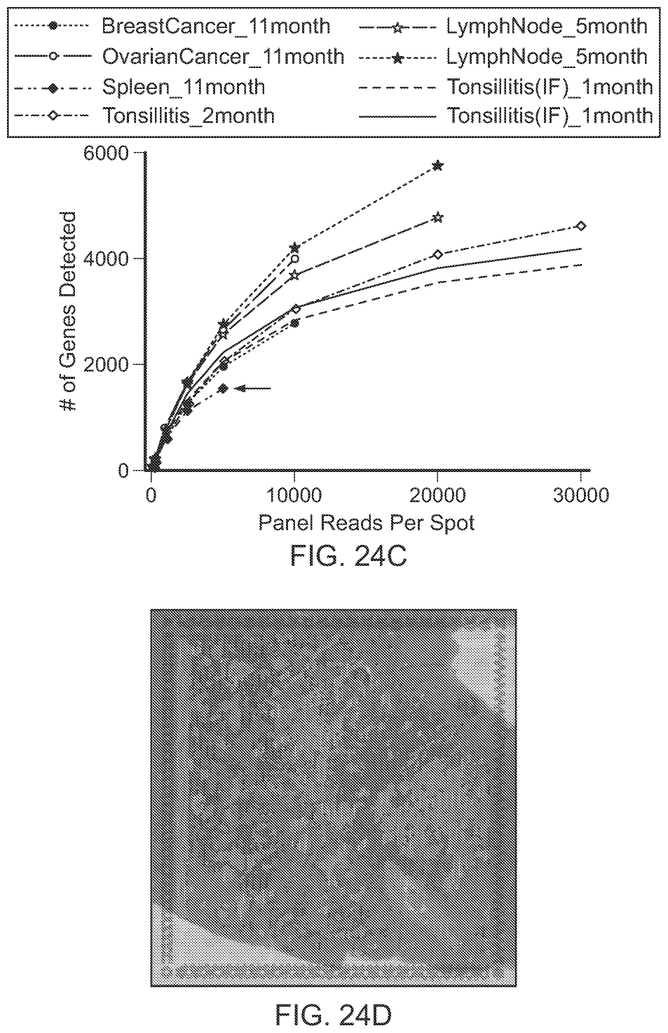

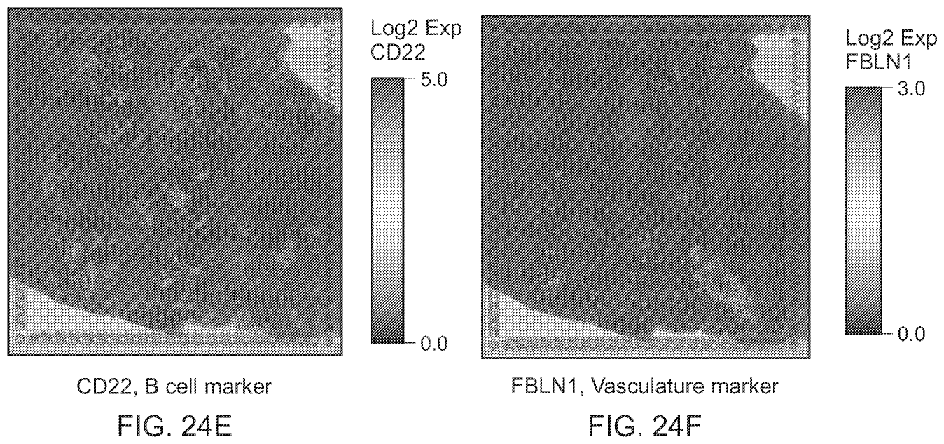

Representative images of the human spleen sample are shown in FIGS. 24A and 24D-24F. For instance, a representative H&E stain is shown in FIG. 24A. Using the methods of spatial analyte detection disclosed herein, one can detect groups of analytes and even individual analytes in a tissue section. As shown in FIG. 24D, UMAP visualization of expression in the human spleen sample reveals 10 different clusters of cell types. The methods disclosed herein also allow for individual analyte expression to reveal different types of cells expressed in a human spleen tissue sample. For instance, as shown in FIGS. 24E-24F, CD22 (FIG. 24E) was expressed in B cells; and FBLN1 (FIG. 24F) was expressed in vasculature cells. Spatial fragment distribution value (DV) at various thresholds (x-axis) compared to spatial fragment DV score (y-axis) in archived sections stored at RT are also shown in FIG. 24B, while sequencing reads per spot compared to number of genes detected per spot in archived sections stored at room temperature (RT) are shown in FIG. 24C. Taken together, these data show that RNA from FFPE human spleen samples stored for months at room temperature can be evaluated using spatial methods described herein.

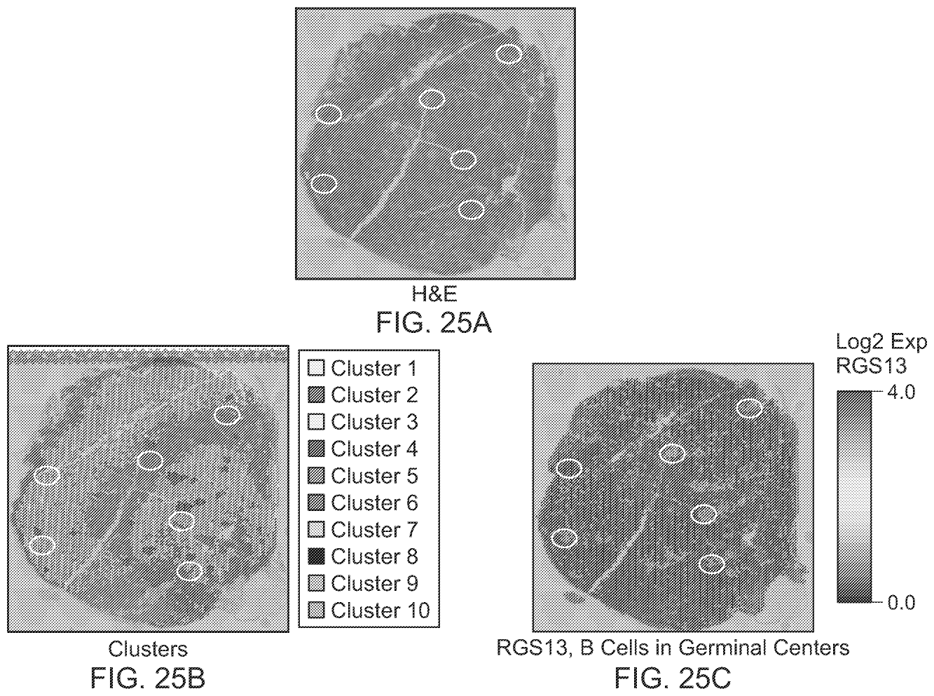

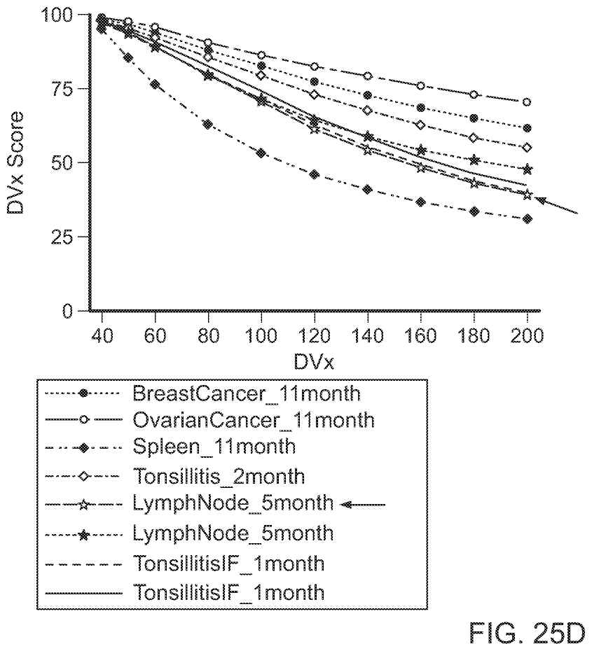

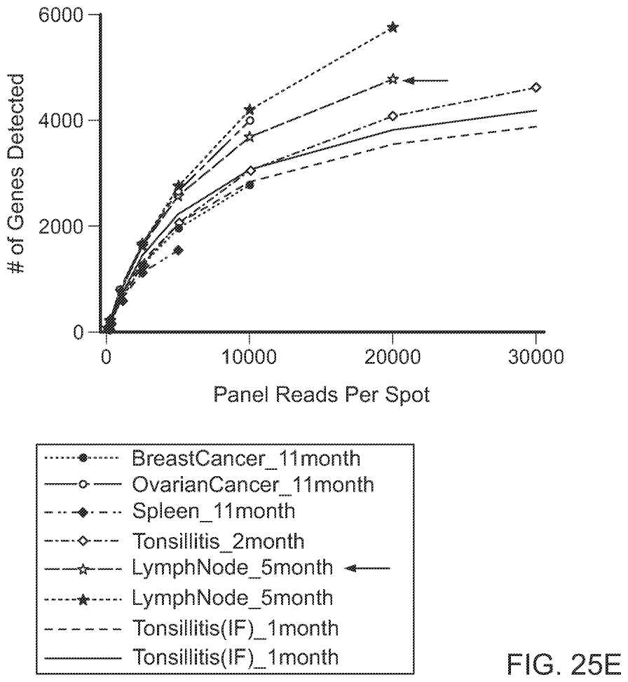

Representative images of the human lymph node sample are shown in FIGS. 25A-25C. For instance, a representative H&E stain is shown in FIG. 25A. Using the methods of spatial analyte detection disclosed herein, one can detect groups of analytes and even individual analytes in a tissue section. As shown in FIG. 25B, UMAP visualization of expression in the human lymph node sample reveals 10 different clusters of cell types. The methods disclosed herein also allow for individual analyte expression to reveal different types of cells expressed in a human lymph node tissue sample. For instance, as shown in FIG. 25C, RGS13 was expressed in B cells in germinal centers. Spatial fragment DV at various thresholds (x-axis) compared to spatial fragment DV score (y-axis) in archived sections stored at room temperature are also shown in FIG. 25D, while sequencing reads per spot compared to number of genes detected per spot in archived sections stored at room temperature are shown in FIG. 25E. Taken together, these data show that RNA from FFPE human lymph node samples stored for months at room temperature can be evaluated using spatial methods described herein.

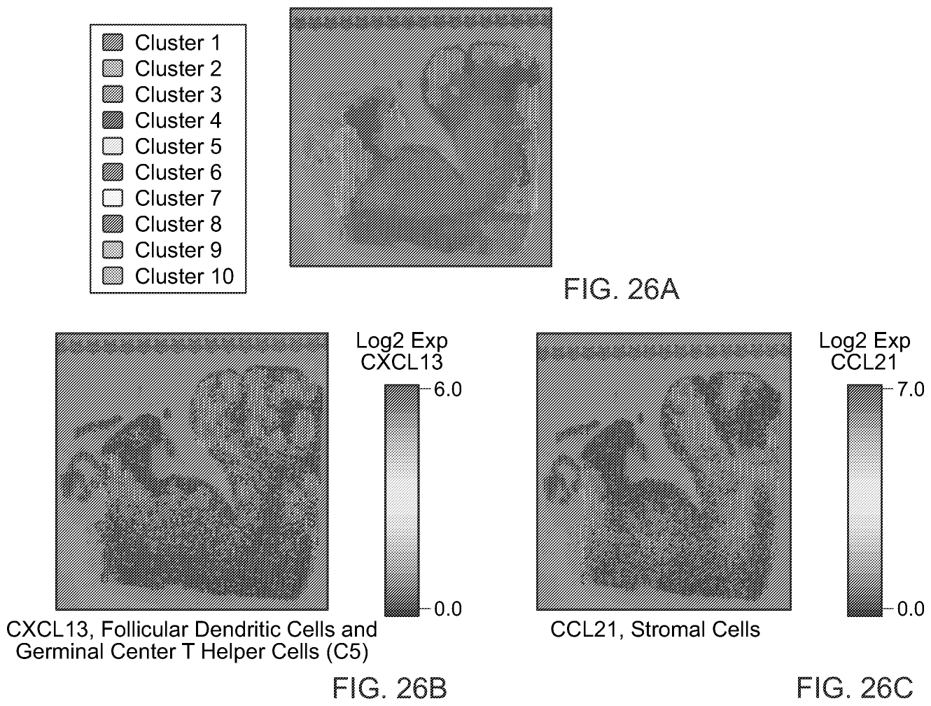

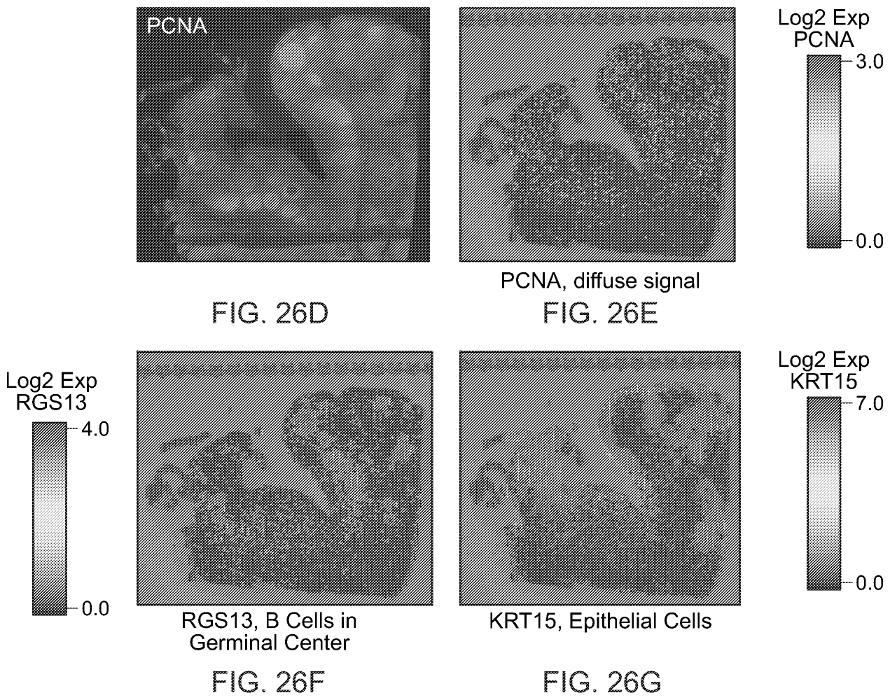

Representative images of the human tonsillitis sample are shown in FIGS. 26A-26G. Using the methods of spatial analyte detection disclosed herein, one can detect groups of analytes and even individual analytes in a tissue section. As shown in FIG. 26A, UMAP visualization of expression in the human tonsillitis sample reveals 10 different clusters of cell types. The methods disclosed herein also allow for individual analyte expression to reveal different types of cells expressed in a human tonsillitis tissue sample. For instance, as shown in FIGS. 26B-26C and 26E-26G, CXCL13 (FIG. 26B) was expressed in follicular dendritic cells and germinal center T helper cells (differentially expressed in Cluster 5 of FIG. 26A); CCL21 (FIG. 26C) was expressed in stromal cells; PCNA (FIG. 26E) was expressed as a diffuse signal; RGS13 (FIG. 26F) was expressed in B cells in germinal center; and KRT15 (FIG. 26G) was expressed in epithelial cells. The human tonsillitis sample was also stained using immunofluorescent labelling of PCNA (FIG. 26D). Taken together, these data show that RNA from FFPE human tonsillitis samples stored for months at room temperature can be evaluated using spatial methods described herein.

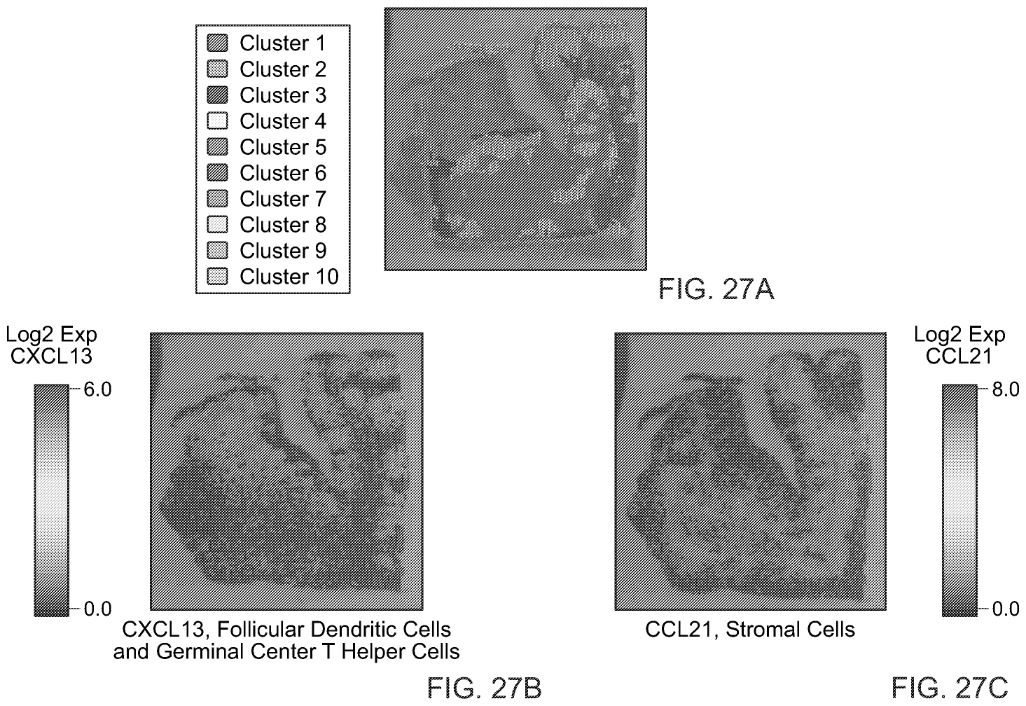

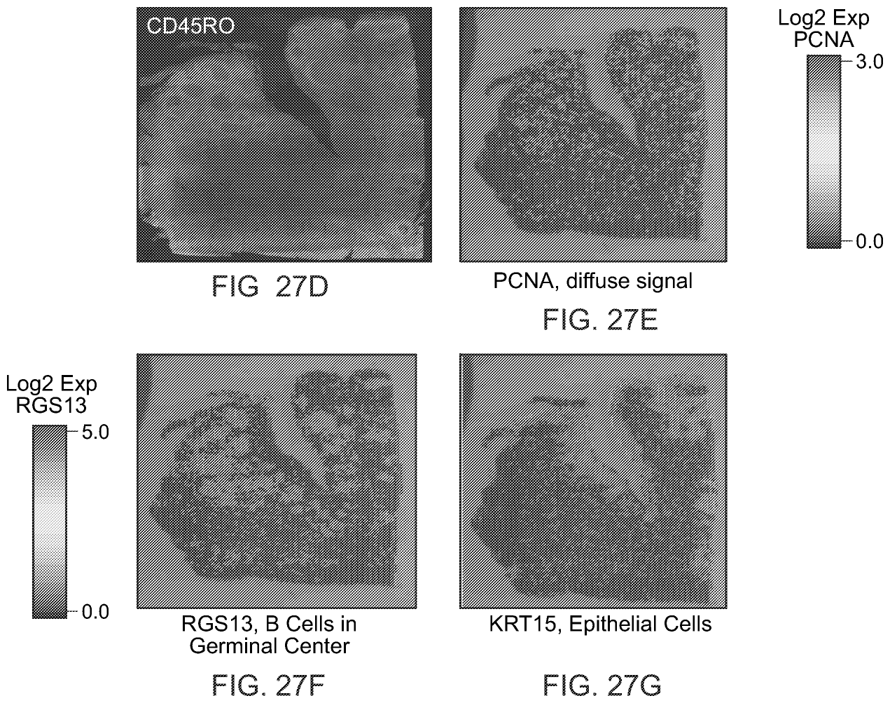

Representative images of another human tonsillitis sample are shown in FIGS. 27A-27G. Using the methods of spatial analyte detection disclosed herein, one can detect groups of analytes and even individual analytes in a tissue section. As shown in FIG. 27A, UMAP visualization of expression in the human tonsillitis sample reveals 10 different clusters of cell types. The methods disclosed herein also allow for individual analyte expression to reveal different types of cells expressed in a human tonsillitis tissue sample. For instance, as shown in FIGS. 27B-27C and 27E-27G, CXCL13 (FIG. 27B) was expressed in follicular dendritic cells and germinal center T helper cells; CCL21 (FIG. 27C) was expressed in stromal cells; PCNA (FIG. 27E) was expressed as a diffuse signal; RGS13 (FIG. 27F) was expressed in B cells in germinal center; and KRT15 (FIG. 27G) was expressed in epithelial cells. The human tonsillitis sample was also stained using immunofluorescent labelling of PCNA (FIG. 27D). Taken together, these data show that RNA from FFPE human tonsillitis samples stored for months at room temperature can be evaluated using spatial methods described herein.

These results collectively demonstrate that spatial methods can identify one or more genes using archived FFPE tissue samples that have been stored for a period of time at room temperature and not in cold storage. Thus, as demonstrated herein. FFPE archived samples that have been stored in less than optimal conditions, can still yield usable data for research and discovery efforts.

1. A method of analyzing an analyte in a fixed biological sample on a first substrate, wherein the fixed biological sample has been affixed to the first substrate for at least four months, the method comprising:
(a) hybridizing a first probe and a second probe to the analyte of the fixed biological sample affixed to the first substrate for at least four months, wherein the first probe and the second probe each comprise a sequence that is substantially complementary to a first sequence and a second sequence of the analyte, respectively, and wherein the second probe further comprises a capture probe binding domain;
(b) coupling the first probe and the second probe, thereby generating a connected probe;
(c) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain;
(d) when the biological sample is aligned with at least a portion of the array, (i) releasing the connected probe from the analyte and (ii) migrating the connected probe from the biological sample to the array; and
(e) hybridizing the connected probe to the capture domain.

2.-4. (canceled)

5. The method of claim 1, wherein the fixed biological sample has been affixed to the first substrate for at least six months, at least one year, at least two years, or at least three years.

6.-10. (canceled)

11. The method of claim 1, wherein the fixed biological sample has been affixed to the first substrate at or above room temperature.

12.-14. (canceled)

15. The method of claim 1 further comprising determining the presence or absence of RNA of sufficient integrity by determining a spatial fragment distribution value (DV) number of the portion of the fixed biological sample.

16. The method of claim 15, wherein the spatial fragment DV number of about 30, about 40, about 50, about 60, about 70, or greater is indicative of the presence of RNA of sufficient integrity.

17. The method of claim 15, wherein determining the presence of RNA of sufficient integrity comprises determining an RNA integrity number (RIN) score of the portion of the fixed biological sample.

18. The method of claim 17, wherein the RIN score of 6 or greater is indicative of the presence of RNA of sufficient integrity.

19.-21. (canceled)

22. The method of claim 1, wherein the fixed biological sample is an FFPE tissue section, a PFA fixed tissue section, or an acetone fixed tissue section.

23-25. (canceled)

26. The method of claim 1, wherein the first sequence and the second sequence are adjacent sequences of the analyte.

27. The method of claim 1, wherein the first sequence and the second sequence are not adjacent to each other on the analyte.

28. The method of claim 27, further comprising:
(i) extending the first probe to generate an extended first probe, thereby filling a gap between the hybridized first probe and the hybridized second probe; or
(ii) extending the second probe to generate an extended second probe, thereby filling a gap between the hybridized first probe and the hybridized second probe.

29.-31. (canceled)

32. The method of claim 1, wherein the coupling of the first probe and the second probe comprises ligating the first probe and the second probe, wherein the ligating comprises use of a ligase, wherein the ligase is selected from a Chlorella virus DNA ligase, a single stranded DNA ligase, or a T4 DNA ligase.

33.-36. (canceled)

37. The method of claim 1, wherein the fixed biological sample is in contact with a reagent medium comprising a permeabilization agent and an agent for releasing the connected probe during the releasing step, thereby permeabilizing the fixed biological sample and releasing the connected probe from the analyte,
wherein the agent for releasing the connected probe comprises a nuclease selected from RNase A, RNase C, RNase H, or RNase I, and
wherein the permeabilization agent comprises a protease selected from trypsin, pepsin, elastase, or proteinase K.

38.-49. (canceled)

50. The method of claim 1, further comprising determining (i) all or a part of the sequence of the connected probe, or a complement thereof, and (ii) the sequence of the spatial barcode, or a complement thereof, wherein the method further comprises using the determined sequence of (i) and (ii) to determine the location of the analyte in the biological sample.

51.-55. (canceled)

56. The method of claim 1, wherein the analyte comprises RNA.

57. (canceled)

58. The method of claim 56, further comprising analyzing a protein analyte in the biological sample.

59.-64. (canceled)

65. The method of claim 1, wherein the hybridizing of the first probe and the second probe to the analyte comprises contacting the fixed biological sample with a set of probe pairs, wherein a probe pair of the set of probe pairs comprises the first probe and the second probe.

66.-99. (canceled)
